(19) United States
(12) Patent Application Publication
(10) Pub. No.: US 2024/0271113 A1
(43) Pub. Date: Aug. 15, 2024

(54) EFFECTOR PROTEINS AND METHODS OF USE

(71) Applicant: Mammoth Biosciences, Inc., Brisbane, CA (US)

(72) Inventors: Lucas Benjamin HARRINGTON, San Francisco, CA (US); David PAEZ-ESPINO, Concord, CA (US); Benjamin Julius RAUCH, San Francisco, CA (US); Stepan TYMOSHENKO, Sacramento, CA (US)

(73) Assignee: Mammoth Biosciences, Inc., Brisbane, CA (US)

(21) Appl. No.: 18/585,306

(22) Filed: Feb. 23, 2024

Related U.S. Application Data
(63) Continuation of application No. PCT/US2022/075228, filed on Aug. 19, 2022.
(60) Provisional application No. 63/285,946, filed on Dec. 3, 2021.
(60) Provisional application No. 63/238,015, filed on Aug. 27, 2021.

(51) Int. Cl. C12N 9/22; A61K 38/00; C12N 15/11; C12N 15/86

(52) U.S. Cl. CPC C12N 9/22; A61K 38/00; C12N 15/111; C12N 15/86; C07K 2319/09; C12N 2750/14143

(57) ABSTRACT

Provided herein are compositions, systems, and methods comprising effector proteins and uses thereof. These effector proteins may be characterized as CRISPR-associated (Cas) proteins. Various compositions, systems, and methods of the present disclosure may leverage the activities of these effector proteins for the modification, detection, and engineering of nucleic acids.

### CROSS-REFERENCE TO RELATED APPLICATIONS

This application is a continuation of International Application No. PCT/US2022/075228, filed Aug. 18, 2022, which claims priority to U.S. Provisional Patent Application No. 63/238,015, filed Aug. 27, 2021, and U.S. Provisional Patent Application No. 63/285,946, filed Dec. 3, 2021, which are incorporated by reference herein in their entirety for all purposes.

### SEQUENCE LISTING

The contents of the electronic sequence listing (MABI_019_02US_SeqList_ST26.xml; Size: 5,853,407 bytes; and Date of Creation: Dec. 12, 2023) are herein incorporated by reference in its entirety.

### BACKGROUND

Programmable nucleases are proteins that bind and cleave nucleic acids in a sequence-specific manner. A programmable nuclease may bind a target sequence of a nucleic acid and cleave the nucleic acid within the target sequence or at a position adjacent to the target sequence. In some instances, a programmable nuclease is activated when it binds a target sequence of a nucleic acid to cleave regions of the nucleic acid that are near, but not adjacent to the target sequence. A programmable nuclease, such as a clustered regularly interspaced short palindromic repeats (CRISPR)-associated (Cas) protein, may be coupled to a guide nucleic acid that imparts activity or sequence selectivity to the programmable nuclease. In general, guide nucleic acids comprise a CRISPR RNA (crRNA) that is at least partially complementary to a target nucleic acid. In some cases, guide nucleic acids comprise a trans-activating crRNA (tracrRNA), at least a portion of which interacts with the programmable nuclease. A guide nucleic acid comprising a crRNA and a tracrRNA is often referred to as a single guide nucleic acid or single guide RNA (sgRNA). In some cases, a tracrRNA is provided separately from the guide nucleic acid. The tracrRNA may hybridize to a portion of the guide nucleic acid that does not hybridize to the target nucleic acid.

Programmable nucleases may cleave nucleic acids, including single stranded RNA (ssRNA), double stranded DNA (dsDNA), and single-stranded DNA (ssDNA). Programmable nucleases may provide cis cleavage activity, trans cleavage activity, nickase activity, or a combination thereof. Cis cleavage activity is cleavage of a target nucleic acid that is hybridized to a guide nucleic acid, wherein cleavage occurs within or directly adjacent to the region of the target nucleic acid that is hybridized to the guide nucleic acid. Trans cleavage activity (also referred to as transcollateral cleavage) is cleavage of ssDNA or ssRNA that is near, but not hybridized to the guide nucleic acid. Trans cleavage activity is triggered by the hybridization of a guide nucleic acid to the target nucleic acid. Nickase activity is the selective cleavage of one strand of a dsDNA molecule. While certain programmable nucleases may be used to edit and detect nucleic acid molecules in a sequence specific manner, challenging biological sample conditions (e.g., high viscosity, metal chelating) may limit their accuracy and effectiveness. There is thus a need for systems and methods that employ programmable nucleases having specificity and efficiency across a wide range of sample conditions.

### SUMMARY

The present disclosure provides polypeptides, such as effector proteins, compositions, and systems comprising the same and uses thereof. In some instances, compositions, systems, and methods comprise guide nucleic acids or uses thereof. Compositions, systems and methods disclosed herein may leverage the nucleic acid modifying activities. Nucleic acid modifying activities may include cis cleavage activity, trans cleavage activity, nicking activity, or nucleobase modifying activity. In some instances, compositions, systems and methods are useful for the detection of target nucleic acids. In some instances, compositions, systems and methods are useful for the treatment of a disease or disorder. The disease or disorder may be associated with one or more mutations in the target nucleic acid.

### I. Certain Embodiments

Disclosed herein, in some aspects, are compositions that comprise an effector protein and an engineered guide nucleic acid, wherein the effector protein comprises an amino acid sequence that is at least 75%, at least 80%, at least 85%, at least 90%, at least 95%, at least 98%, at least 99% or 100% identical to any one of SEQ ID NOs: 1-1614 and 3195-3302. Also, disclosed herein, in some aspects, are compositions that comprise an effector protein and an engineered guide nucleic acid, wherein the amino acid sequence of the effector protein is at least 75%, at least 80%, at least 85%, at least 90%, at least 95%, at least 98%, at least 99% or 100% identical to any one of SEQ ID NOs: 1-1614 and 3195-3302. Also, disclosed herein, in some aspects, are compositions that comprise an effector protein and an engineered guide nucleic acid, wherein the effector protein comprises about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, about 840, about 860, about 880, about 900, about 920, about 940, about 960, about 980, about 1000, about 1020, about 1040, about 1060, about 1080, about 1100, about 1120, about 1140, about 1160, about 1180, about 1200, about 1220, about 1240, about 1260, about 1280, about 1300, about 1320, about 1340, about 1360, about 1380, or about 1400 contiguous amino acids of a sequence selected from any one of SEQ ID NOS: 1-1614 and 3195-3302. Also, disclosed herein, in some aspects, are compositions that comprise an effector protein and an engineered guide nucleic acid, wherein the effector protein comprises the amino acid sequence located at positions 1-100, 150-250, 101-200, 250-350, 201-300, 350-450, 301-400, 350-450, 401-500, 450-550, 501-600, 550-650, 601-700, 650-750, 701-800, 750-850, 801-900, 850-950, 901-1000, 950-1050, 1001-1100, 1050-1150, 1101-1200, 1150-1250, 1201-1300, 1250-1350, 1301-1400, 1350-1450, 1401-1500, 1450-1550, 1501-1600, 1550-1650, 1601-1700, 1650-1750, 1701-1800, 1850-1950, or 1801-1900 of a sequence selected from SEQ ID NOS: 1-1614 and 3195-3302. Also, disclosed herein, in some aspects, are compositions that comprise an effector protein and an engineered guide nucleic acid, wherein the effector protein comprises an amino acid sequence that is at least 90%, at least 95%, or 100% identical to a portion of a sequence selected from SEQ ID NOS: 1-1614 and 3195-3302, wherein the portion of the sequence is about 30%, about 40% about 50%, about 60%, about 70%, about 80%, or about 90% of the sequence. Also, disclosed herein, in some aspects, are compositions that comprise an effector protein, and an engineered guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A1 of TABLE 1; and at least a portion of the engineered guide nucleic acid comprises a nucleobase sequence that is: i) at least 50%, at least 55%, at least 60%, at least 65%, at least 70%, at least 75%, at least 80%, at least 85%, at least 90%, at least 95%, or 100% identical to a sequence selected from Column B1 of TABLE 1, wherein the sequence from Column A1 and the sequence from Column B1 are in the same row of TABLE 1, or ii) at least 50%, at least 55%, at least 60%, at least 65%, at least 70%, at least 75%, at least 80%, at least 85%, at least 90%, at least 95%, or 100% reverse complementary to a sequence selected from Column B1 of TABLE 1, wherein the sequence from Column A1 and the sequence from Column B1 are in the same row of TABLE 1. Also, disclosed herein, in some aspects, are compositions that comprise an effector protein, and an engineered guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A2 of TABLE 1; and at least a portion of the engineered guide nucleic acid comprises a nucleobase sequence that is: i) at least 50%, at least 55%, at least 60%, at least 65%, at least 70%, at least 75%, at least 80%, at least 85%, at least 90%, at least 95%, or 100% identical to a sequence selected from Column B2 of TABLE 1, wherein the sequence from Column A2 and the sequence from Column B2 are in the same row of TABLE 1, or ii) at least 50%, at least 55%, at least 60%, at least 65%, at least 70%, at least 75%, at least 80%, at least 85%, at least 90%, at least 95%, or 100% reverse complementary to a sequence selected from Column B2 of TABLE 1, wherein the sequence from Column A2 and the sequence from Column B2 are in the same row of TABLE 1. Also, disclosed herein, in some aspects, are compositions that comprise an effector protein, and an engineered guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A3 of TABLE 1; and at least a portion of the engineered guide nucleic acid comprises a nucleobase sequence that is: i) at least 50%, at least 55%, at least 60%, at least 65%, at least 70%, at least 75%, at least 80%, at least 85%, at least 90%, at least 95%, or 100% identical to a sequence selected from Column B3 of TABLE 1, wherein the sequence from Column A3 and the sequence from Column B3 are in the same row of TABLE 1, or ii) at least 50%, at least 55%, at least 60%, at least 65%, at least 70%, at least 75%, at least 80%, at least 85%, at least 90%, at least 95%, or 100% reverse complementary to a sequence selected from Column B3 of TABLE 1, wherein the sequence from Column A3 and the sequence from Column B3 are in the same row of TABLE 1.

Also, disclosed herein, in some aspects, are compositions that comprise an effector protein, and an engineered guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column C1 of TABLE 2; and at least a portion of the engineered guide nucleic acid comprises a nucleobase sequence that is: i) at least 50%, at least 55%, at least 60%, at least 65%, at least 70%, at least 75%, at least 80%, at least 85%, at least 90%, at least 95%, or 100% identical to a sequence selected from Column D1 of TABLE 2, wherein the sequence from Column C1 and the sequence from Column D1 are in the same row of TABLE 2, or ii) at least 50%, at least 55%, at least 60%, at least 65%, at least 70%, at least 75%, at least 80%, at least 85%, at least 90%, at least 95%, or 100% reverse complementary to a sequence selected from Column D1 of TABLE 2, wherein the sequence from Column C1 and the sequence from Column D1 are in the same row of TABLE 2. Also, disclosed herein, in some aspects, are compositions that comprise an effector protein, and an engineered guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column C2 of TABLE 2; and at least a portion of the engineered guide nucleic acid comprises a nucleobase sequence that is: i) at least 50%, at least 55%, at least 60%, at least 65%, at least 70%, at least 75%, at least 80%, at least 85%, at least 90%, at least 95%, or 100% identical to a sequence selected from Column D2 of TABLE 2, wherein the sequence from Column C2 and the sequence from Column D2 are in the same row of TABLE 2, or ii) at least 50%, at least 55%, at least 60%, at least 65%, at least 70%, at least 75%, at least 80%, at least 85%, at least 90%, at least 95%, or 100% reverse complementary to a sequence selected from Column D2 of TABLE 2, wherein the sequence from Column C2 and the sequence from Column D2 are in the same row of TABLE 2.

Also, disclosed herein, in some aspects, are compositions that comprise an effector protein, and an engineered guide nucleic acid, wherein the amino acid sequence of the effector protein is at least 50% identical to a sequence selected from Column A1 of TABLE 1, and wherein at least a portion of the engineered guide nucleic acid comprises a nucleobase sequence that is at least 50% identical or at least 50% reverse complementary to a sequence selected from Column B1 of TABLE 1, wherein the sequence from Column A1 and the sequence from Column B1 are in the same row of TABLE 1. Also, disclosed herein, in some aspects, are compositions that comprise an effector protein, and an engineered guide nucleic acid, wherein the amino acid sequence of the effector protein is at least 60% identical to a sequence selected from Column A1 of TABLE 1, and wherein at least a portion of the engineered guide nucleic acid comprises a nucleobase sequence that is at least 60% identical or at least 60% reverse complementary to a sequence selected from Column B1 of TABLE 1, wherein the sequence from Column A1 and the sequence from Column B1 are in the same row of TABLE 1. Also, disclosed herein, in some aspects, are compositions that comprise an effector protein, and an engineered guide nucleic acid, wherein the amino acid sequence of the effector protein is at least 70% identical to a sequence selected from Column A1 of TABLE 1, and wherein at least a portion of the engineered guide nucleic acid comprises a nucleobase sequence that is at least 70% identical or at least 70% reverse complementary to a sequence selected from Column B1 of TABLE 1, wherein the sequence from Column A1 and the sequence from Column B1 are in the same row of TABLE 1. Also, disclosed herein, in some aspects, are compositions that comprise an effector protein, and an engineered guide nucleic acid, wherein the amino acid sequence of the effector protein is at least 80% identical to a sequence selected from Column A1 of TABLE 1, and wherein at least a portion of the engineered guide nucleic acid comprises a nucleobase sequence that is at least 80% identical or at least 80% reverse complementary to a sequence selected from Column B1 of TABLE 1, wherein the sequence from Column A1 and the sequence from Column B1 are in the same row of TABLE 1. Also, disclosed herein, in some aspects, are compositions that comprise an effector protein, and an engineered guide nucleic acid, wherein the amino acid sequence of the effector protein is at least 90% identical to a sequence selected from Column A1 of TABLE 1, and wherein at least a portion of the engineered guide nucleic acid comprises a nucleobase sequence that is at least 90% identical or at least 90% reverse complementary to a sequence selected from Column B1 of TABLE 1, wherein the sequence from Column A1 and the sequence from Column B1 are in the same row of TABLE 1. Also, disclosed herein, in some aspects, are compositions that comprise an effector protein, and an engineered guide nucleic acid, wherein the amino acid sequence of the effector protein is at least 95% identical to a sequence selected from Column A1 of TABLE 1, and wherein at least a portion of the engineered guide nucleic acid comprises a nucleobase sequence that is at least 95% identical or at least 95% reverse complementary to a sequence selected from Column B1 of TABLE 1, wherein the sequence from Column A1 and the sequence from Column B1 are in the same row of TABLE 1. Also, disclosed herein, in some aspects, are compositions that comprise an effector protein, and an engineered guide nucleic acid, wherein the amino acid sequence of the effector protein is 100% identical to a sequence selected from Column A1 of TABLE 1, and wherein at least a portion of the engineered guide nucleic acid comprises a nucleobase sequence that is 100% identical or 100% reverse complementary to a sequence selected from Column B1 of TABLE 1, wherein the sequence from Column A1 and the sequence from Column B1 are in the same row of TABLE 1. Also, disclosed herein, in some aspects, are compositions that comprise an effector protein, and an engineered guide nucleic acid, wherein the amino acid sequence of the effector protein is at least 50% identical to a sequence selected from Column A2 of TABLE 1, and wherein at least a portion of the engineered guide nucleic acid comprises a nucleobase sequence that is at least 50% identical or at least 50% reverse complementary to a sequence selected from Column B2 of TABLE 1, wherein the sequence from Column A2 and the sequence from Column B2 are in the same row of TABLE 1. Also, disclosed herein, in some aspects, are compositions that comprise an effector protein, and an engineered guide nucleic acid, wherein the amino acid sequence of the effector protein is at least 60% identical to a sequence selected from Column A2 of TABLE 1, and wherein at least a portion of the engineered guide nucleic acid comprises a nucleobase sequence that is at least 60% identical or at least 60% reverse complementary to a sequence selected from Column B2 of TABLE 1, wherein the sequence from Column A2 and the sequence from Column B2 are in the same row of TABLE 1. Also, disclosed herein, in some aspects, are compositions that comprise an effector protein, and an engineered guide nucleic acid, wherein the amino acid sequence of the effector protein is at least 70% identical to a sequence selected from Column A2 of TABLE 1, and wherein at least a portion of the engineered guide nucleic acid comprises a nucleobase sequence that is at least 70% identical or at least 70% reverse complementary to a sequence selected from Column B2 of TABLE 1, wherein the sequence from Column A2 and the sequence from Column B2 are in the same row of TABLE 1. Also, disclosed herein, in some aspects, are compositions that comprise an effector protein, and an engineered guide nucleic acid, wherein the amino acid sequence of the effector protein is at least 80% identical to a sequence selected from Column A2 of TABLE 1, and wherein at least a portion of the engineered guide nucleic acid comprises a nucleobase sequence that is at least 80% identical or at least 80% reverse complementary to a sequence selected from Column B2 of TABLE 1, wherein the sequence from Column A2 and the sequence from Column B2 are in the same row of TABLE 1. Also, disclosed herein, in some aspects, are compositions that comprise an effector protein, and an engineered guide nucleic acid, wherein the amino acid sequence of the effector protein is at least 90% identical to a sequence selected from Column A2 of TABLE 1, and wherein at least a portion of the engineered guide nucleic acid comprises a nucleobase sequence that is at least 90% identical or at least 90% reverse complementary to a sequence selected from Column B2 of TABLE 1, wherein the sequence from Column A2 and the sequence from Column B2 are in the same row of TABLE 1. Also, disclosed herein, in some aspects, are compositions that comprise an effector protein, and an engineered guide nucleic acid, wherein the amino acid sequence of the effector protein is at least 95% identical to a sequence selected from Column A2 of TABLE 1, and wherein at least a portion of the engineered guide nucleic acid comprises a nucleobase sequence that is at least 95% identical or at least 95% reverse complementary to a sequence selected from Column B2 of TABLE 1, wherein the sequence from Column A2 and the sequence from Column B2 are in the same row of TABLE 1. Also, disclosed herein, in some aspects, are compositions that comprise an effector protein, and an engineered guide nucleic acid, wherein the amino acid sequence of the effector protein is 100% identical to a sequence selected from Column A2 of TABLE 1, and wherein at least a portion of the engineered guide nucleic acid comprises a nucleobase sequence that is 100% identical or 100% reverse complementary to a sequence selected from Column B2 of TABLE 1, wherein the sequence from Column A2 and the sequence from Column B2 are in the same row of TABLE 1. Also, disclosed herein, in some aspects, are compositions that comprise an effector protein, and an engineered guide nucleic acid, wherein the amino acid sequence of the effector protein is at least 50% identical to a sequence selected from Column A3 of TABLE 1, and wherein at least a portion of the engineered guide nucleic acid comprises a nucleobase sequence that is at least 50% identical or at least 50% reverse complementary to a sequence selected from Column B3 of TABLE 1, wherein the sequence from Column A3 and the sequence from Column B3 are in the same row of TABLE 1. Also, disclosed herein, in some aspects, are compositions that comprise an effector protein, and an engineered guide nucleic acid, wherein the amino acid sequence of the effector protein is at least 60% identical to a sequence selected from Column A3 of TABLE 1, and wherein at least a portion of the engineered guide nucleic acid comprises a nucleobase sequence that is at least 60% identical or at least 60% reverse complementary to a sequence selected from Column B3 of TABLE 1, wherein the sequence from Column A3 and the sequence from Column B3 are in the same row of TABLE 1. Also, disclosed herein, in some aspects, are compositions that comprise an effector protein, and an engineered guide nucleic acid, wherein the amino acid sequence of the effector protein is at least 70% identical to a sequence selected from Column A3 of TABLE 1, and wherein at least a portion of the engineered guide nucleic acid comprises a nucleobase sequence that is at least 70% identical or at least 70% reverse complementary to a sequence selected from Column B3 of TABLE 1, wherein the sequence from Column A3 and the sequence from Column B3 are in the same row of TABLE 1. Also, disclosed herein, in some aspects, are compositions that comprise an effector protein, and an engineered guide nucleic acid, wherein the amino acid sequence of the effector protein is at least 80% identical to a sequence selected from Column A3 of TABLE 1, and wherein at least a portion of the engineered guide nucleic acid comprises a nucleobase sequence that is at least 80% identical or at least 80% reverse complementary to a sequence selected from Column B3 of TABLE 1, wherein the sequence from Column A3 and the sequence from Column B3 are in the same row of TABLE 1. Also, disclosed herein, in some aspects, are compositions that comprise an effector protein, and an engineered guide nucleic acid, wherein the amino acid sequence of the effector protein is at least 90% identical to a sequence selected from Column A3 of TABLE 1, and wherein at least a portion of the engineered guide nucleic acid comprises a nucleobase sequence that is at least 90% identical or at least 90% reverse complementary to a sequence selected from Column B3 of TABLE 1, wherein the sequence from Column A3 and the sequence from Column B3 are in the same row of TABLE 1. Also, disclosed herein, in some aspects, are compositions that comprise an effector protein, and an engineered guide nucleic acid, wherein the amino acid sequence of the effector protein is at least 95% identical to a sequence selected from Column A3 of TABLE 1, and wherein at least a portion of the engineered guide nucleic acid comprises a nucleobase sequence that is at least 95% identical or at least 95% reverse complementary to a sequence selected from Column B3 of TABLE 1, wherein the sequence from Column A3 and the sequence from Column B3 are in the same row of TABLE 1. Also, disclosed herein, in some aspects, are compositions that comprise an effector protein, and an engineered guide nucleic acid, wherein the amino acid sequence of the effector protein is 100% identical to a sequence selected from Column A3 of TABLE 1, and wherein at least a portion of the engineered guide nucleic acid comprises a nucleobase sequence that is 100% identical or 100% reverse complementary to a sequence selected from Column B3 of TABLE 1, wherein the sequence from Column A3 and the sequence from Column B3 are in the same row of TABLE 1.

Also, disclosed herein, in some aspects, are compositions that comprise an effector protein, and an engineered guide nucleic acid, wherein the amino acid sequence of the effector protein is at least 50% identical to a sequence selected from Column C1 of TABLE 2, and wherein at least a portion of the engineered guide nucleic acid comprises a nucleobase sequence that is at least 50% identical or at least 50% reverse complementary to a sequence selected from Column D1 of TABLE 2, wherein the sequence from Column C1 and the sequence from Column D1 are in the same row of TABLE 2. Also, disclosed herein, in some aspects, are compositions that comprise an effector protein, and an engineered guide nucleic acid, wherein the amino acid sequence of the effector protein is at least 60% identical to a sequence selected from Column C1 of TABLE 2, and wherein at least a portion of the engineered guide nucleic acid comprises a nucleobase sequence that is at least 60% identical or at least 60% reverse complementary to a sequence selected from Column D1 of TABLE 2, wherein the sequence from Column C1 and the sequence from Column D1 are in the same row of TABLE 2. Also, disclosed herein, in some aspects, are compositions that comprise an effector protein, and an engineered guide nucleic acid, wherein the amino acid sequence of the effector protein is at least 70% identical to a sequence selected from Column C1 of TABLE 2, and wherein at least a portion of the engineered guide nucleic acid comprises a nucleobase sequence that is at least 70% identical or at least 70% reverse complementary to a sequence selected from Column D1 of TABLE 2, wherein the sequence from Column C1 and the sequence from Column D1 are in the same row of TABLE 2. Also, disclosed herein, in some aspects, are compositions that comprise an effector protein, and an engineered guide nucleic acid, wherein the amino acid sequence of the effector protein is at least 80% identical to a sequence selected from Column C1 of TABLE 2, and wherein at least a portion of the engineered guide nucleic acid comprises a nucleobase sequence that is at least 80% identical or at least 80% reverse complementary to a sequence selected from Column D1 of TABLE 2, wherein the sequence from Column C1 and the sequence from Column D1 are in the same row of TABLE 2. Also, disclosed herein, in some aspects, are compositions that comprise an effector protein, and an engineered guide nucleic acid, wherein the amino acid sequence of the effector protein is at least 90% identical to a sequence selected from Column C1 of TABLE 2, and wherein at least a portion of the engineered guide nucleic acid comprises a nucleobase sequence that is at least 90% identical or at least 90% reverse complementary to a sequence selected from Column D1 of TABLE 2, wherein the sequence from Column C1 and the sequence from Column D1 are in the same row of TABLE 2. Also, disclosed herein, in some aspects, are compositions that comprise an effector protein, and an engineered guide nucleic acid, wherein the amino acid sequence of the effector protein is at least 95% identical to a sequence selected from Column C1 of TABLE 2, and wherein at least a portion of the engineered guide nucleic acid comprises a nucleobase sequence that is at least 95% identical or at least 95% reverse complementary to a sequence selected from Column D1 of TABLE 2, wherein the sequence from Column C1 and the sequence from Column D1 are in the same row of TABLE 2. Also, disclosed herein, in some aspects, are compositions that comprise an effector protein, and an engineered guide nucleic acid, wherein the amino acid sequence of the effector protein is 100% identical to a sequence selected from Column C1 of TABLE 2, and wherein at least a portion of the engineered guide nucleic acid comprises a nucleobase sequence that is 100% identical or 100% reverse complementary to a sequence selected from Column D1 of TABLE 2, wherein the sequence from Column C1 and the sequence from Column D1 are in the same row of TABLE 2.

Also, disclosed herein, in some aspects, are compositions that comprise an effector protein, and an engineered guide nucleic acid, wherein the amino acid sequence of the effector protein is at least 50% identical to a sequence selected from Column C2 of TABLE 2, and wherein at least a portion of the engineered guide nucleic acid comprises a nucleobase sequence that is at least 50% identical or at least 50% reverse complementary to a sequence selected from Column D2 of TABLE 2, wherein the sequence from Column C2 and the sequence from Column D2 are in the same row of TABLE 2. Also, disclosed herein, in some aspects, are compositions that comprise an effector protein, and an engineered guide nucleic acid, wherein the amino acid sequence of the effector protein is at least 60% identical to a sequence selected from Column C2 of TABLE 2, and wherein at least a portion of the engineered guide nucleic acid comprises a nucleobase sequence that is at least 60% identical or at least 60% reverse complementary to a sequence selected from Column D2 of TABLE 2, wherein the sequence from Column C2 and the sequence from Column D2 are in the same row of TABLE 2. Also, disclosed herein, in some aspects, are compositions that comprise an effector protein, and an engineered guide nucleic acid, wherein the amino acid sequence of the effector protein is at least 70% identical to a sequence selected from Column C2 of TABLE 2, and wherein at least a portion of the engineered guide nucleic acid comprises a nucleobase sequence that is at least 70% identical or at least 70% reverse complementary to a sequence selected from Column D2 of TABLE 2, wherein the sequence from Column C2 and the sequence from Column D2 are in the same row of TABLE 2. Also, disclosed herein, in some aspects, are compositions that comprise an effector protein, and an engineered guide nucleic acid, wherein the amino acid sequence of the effector protein is at least 80% identical to a sequence selected from Column C2 of TABLE 2, and wherein at least a portion of the engineered guide nucleic acid comprises a nucleobase sequence that is at least 80% identical or at least 80% reverse complementary to a sequence selected from Column D2 of TABLE 2, wherein the sequence from Column C2 and the sequence from Column D2 are in the same row of TABLE 2. Also, disclosed herein, in some aspects, are compositions that comprise an effector protein, and an engineered guide nucleic acid, wherein the amino acid sequence of the effector protein is at least 90% identical to a sequence selected from Column C2 of TABLE 2, and wherein at least a portion of the engineered guide nucleic acid comprises a nucleobase sequence that is at least 90% identical or at least 90% reverse complementary to a sequence selected from Column D2 of TABLE 2, wherein the sequence from Column C2 and the sequence from Column D2 are in the same row of TABLE 2. Also, disclosed herein, in some aspects, are compositions that comprise an effector protein, and an engineered guide nucleic acid, wherein the amino acid sequence of the effector protein is at least 95% identical to a sequence selected from Column C2 of TABLE 2, and wherein at least a portion of the engineered guide nucleic acid comprises a nucleobase sequence that is at least 95% identical or at least 95% reverse complementary to a sequence selected from Column D2 of TABLE 2, wherein the sequence from Column C2 and the sequence from Column D2 are in the same row of TABLE 2. Also, disclosed herein, in some aspects, are compositions that comprise an effector protein, and an engineered guide nucleic acid, wherein the amino acid sequence of the effector protein is 100% identical to a sequence selected from Column C2 of TABLE 2, and wherein at least a portion of the engineered guide nucleic acid comprises a nucleobase sequence that is 100% identical or 100% reverse complementary to a sequence selected from Column D2 of TABLE 2, wherein the sequence from Column C2 and the sequence from Column D2 are in the same row of TABLE 2.

In some embodiments, the portion of the engineered guide nucleic acid binds the effector protein. In some embodiments, the engineered guide nucleic acid comprises a crRNA. In some embodiments, the engineered guide nucleic acid comprises a tracrRNA. In some embodiments, the effector protein comprises a nuclear localization signal. In some embodiments, the length of the effector protein is at least 700, at least 750, at least 800, at least 850, at least 900, at least 950, at least 1000, at least 1050, at least 1100, at least 1150, or at least 1200 linked amino acid residues. In some embodiments, the length of the effector protein is less than about 1900 linked amino acids. In some embodiments, the length of the effector protein is about 700 linked amino acids to about 1900 linked amino acids. In some embodiments, the length of the effector protein is about 700 to about 800, about 800 to about 900, about 900 to about 1000, about 1000 to about 1100, about 1100 to about 1200, about 1200 to about 1300, about 1300 to about 1400, about 1400 to about 1500, about 1500 to about 1600, about 1600 to about 1700, about 1700 to about 1800, or about 1800 to about 1900 linked amino acids. In some embodiments, compositions comprise a donor nucleic acid. In some embodiments, compositions comprise a fusion partner protein. In some embodiments, the fusion partner protein is directly fused to the N terminus or C terminus of the effector protein via an amide bond. In some embodiments, the fusion partner protein is directly fused to the N terminus or C terminus of the effector protein via a linker peptide. In some embodiments, the fusion partner protein comprises a polypeptide selected from a deaminase, a transcriptional activator, a transcriptional repressor, or a functional domain thereof. In some embodiments, the effector protein comprises at least one mutation that reduces its nuclease activity as measured in a standard cleavage assay. In some embodiments, the effector protein is a catalytically inactive nuclease. In some embodiments, the composition is a pharmaceutical composition comprising a pharmaceutically acceptable excipient.

Disclosed herein, in some aspects, are compositions that comprise a nucleic acid expression vector encoding an effector protein, wherein the effector protein comprises an amino acid sequence that is at least 75%, at least 80%, at least 85%, at least 90%, at least 95%, at least 98%, at least 99% or 100% identical to any one of SEQ ID NOs: 1-1614 and 3195-3302. In some embodiments, the nucleic acid expression vector encodes an engineered guide nucleic acid. In some embodiments, compositions comprise an additional nucleic acid expression vector encoding an engineered guide nucleic acid. In some embodiments, the effector protein comprises an amino acid sequence that is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A1, Column A2, and Column A3 of TABLE 1 and Column C1 and C2 of TABLE 2, and wherein at least a portion of the engineered guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column B1, Column B2, and Column B3 of TABLE 1 and Column D1 and D2 of TABLE 2, respectively, wherein the sequence from Column A1, A2, or A3 and the sequence from Column B1, B2, or B3 are in the same row of TABLE 1 and the sequence from Column C1 or C2 and the sequence from Column D1 or D2 are in the same row of TABLE 2, respectively. In some embodiments, compositions comprise a donor nucleic acid. In some embodiments, the donor nucleic acid is encoded by the nucleic acid expression vector or additional nucleic acid expression vector.

Disclosed herein are viruses comprising a nucleic acid expression vector encoding an effector protein, wherein the effector protein comprises an amino acid sequence that is at least 75%, at least 80%, at least 85%, at least 90%, at least 95%, at least 98%, at least 99% or 100% identical to any one of SEQ ID NOs: 1-1614 and 3195-3302. In some embodiments, the nucleic acid expression vector encodes an engineered guide nucleic acid. In some embodiments, compositions comprise an additional nucleic acid expression vector encoding an engineered guide nucleic acid. In some embodiments, the effector protein comprises an amino acid sequence that is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A1, Column A2, and Column A3 of TABLE 1 and Column C1 and C2 of TABLE 2, and wherein at least a portion of the engineered guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column B1, Column B2, and Column B3 of TABLE 1 and Column D1 and D2 of TABLE 2, respectively, wherein the sequence from Column A1, A2, or A3 and the sequence from Column B1, B2, or B3 are in the same row of TABLE 1 and the sequence from Column C1 or C2 and the sequence from Column D1 or D2 are in the same row of TABLE 2, respectively. In some embodiments, compositions comprise a donor nucleic acid. In some embodiments, the donor nucleic acid is encoded by the nucleic acid expression vector or additional nucleic acid expression vector.

Disclosed herein, in some aspects, are systems that comprise a composition described herein and at least one detection reagent for detecting a target nucleic acid. In some embodiments, the at least one detection reagent is selected from a reporter nucleic acid, a detection moiety, an additional effector protein, or a combination thereof, optionally wherein the reporter nucleic acid comprises a fluorophore, a quencher, or a combination thereof. In some embodiments, systems comprise at least one amplification reagent for amplifying a target nucleic acid. In some embodiments, the at least one amplification reagent is selected from the group consisting of a primer, an activator, a dNTP, an rNTP, and combinations thereof.

Disclosed herein, in some aspects, are methods of modifying a target nucleic acid in a sample, comprising contacting the sample with a composition described herein, a virus described herein, a pharmaceutical composition described herein, or a system described herein, thereby generating a modification of the target nucleic acid; and optionally detecting the modification.

Disclosed herein, in some aspects, are methods of detecting a target nucleic acid in a sample, comprising the steps of: contacting the sample with a composition or system described herein, a reporter nucleic acid comprising a detectable moiety that produces a detectable signal in the presence of the target nucleic acid and the composition or system, and detecting the detectable signal. In some embodiments, the reporter nucleic acid comprises a fluorophore, a quencher, or a combination thereof, and wherein the detecting comprises detecting a fluorescent signal. In some embodiments, methods comprise reverse transcribing the target nucleic acid, amplifying the target nucleic acid, in vitro transcribing the target nucleic acid, or any combination thereof. In some embodiments, methods comprise reverse transcribing the target nucleic acid and/or amplifying the target nucleic acid before contacting the sample with the composition. In some embodiments, methods comprise reverse transcribing the target nucleic acid and/or amplifying the target nucleic acid after contacting the sample with the composition. In some embodiments, amplifying comprises isothermal amplification. In some embodiments, the target nucleic acid is from a pathogen. In some embodiments, the pathogen is a virus. In some embodiments, the target nucleic acid comprises RNA. In some embodiments, the target nucleic acid comprises DNA.

Disclosed herein, in some aspects, are methods of modifying a target nucleic acid, the method comprising contacting the target nucleic acid with a composition described herein, a virus described herein, or a pharmaceutical composition described herein, thereby modifying the target nucleic acid. In some embodiments, modifying the target nucleic acid comprises cleaving the target nucleic acid, deleting a nucleotide of the target nucleic acid, inserting a nucleotide into the target nucleic acid, substituting a nucleotide of the target nucleic acid with a donor nucleotide or an additional nucleotide, or any combination thereof. In some embodiments, methods comprise contacting the target nucleic acid with a donor nucleic acid. In some embodiments, the target nucleic acid comprises a mutation associated with a disease. In some embodiments, the disease is suspected to cause, at least in part, a cancer, an inherited disorder, an ophthalmological disorder, or a combination thereof. In some embodiments, the disease is cancer, an ophthalmological disease, a neurological disorder, a blood disorder, or a metabolic disorder. In some embodiments, the neurological disorder is Duchenne muscular dystrophy, myotonic dystrophy Type 1, or cystic fibrosis. In some embodiments, the neurological disorder is a neurodegenerative disease. In some embodiments, contacting the target nucleic acid comprises contacting a cell, wherein the target nucleic acid is located in the cell. In some embodiments, contacting occurs in vitro. In some embodiments, the contacting occurs in vivo. In some embodiments, contacting occurs ex vivo.

Disclosed herein, in some aspects, are cells that comprise a composition described herein. In some embodiments, cells are infected by a virus described herein. Also, disclosed herein, in some aspects, are cells that comprise a modified target nucleic acid, wherein the modified target nucleic acid is a target nucleic acid modified according to any one of the methods described herein. In some embodiments, the cell is a eukaryotic cell. In some embodiments, the cell is a mammalian cell. In some embodiments, the cell is a T cell. In some embodiments, the T cell is a natural killer T cell (NKT). In some embodiments, the cell is an induced pluripotent stem cell (iPSC).

Disclosed herein, in some aspects, are methods of producing a protein, the method comprising, contacting a cell comprising a target nucleic acid to a compositions described herein, thereby editing the target nucleic acid to produce a modified cell comprising a modified target nucleic acid; and producing a protein from the cell that is encoded, transcriptionally affected, or translationally affected by the modified nucleic acid.

Disclosed herein, in some aspects, are methods of treating a disease comprising administering to a subject in need thereof a composition described herein, a virus described herein, a pharmaceutical composition described herein, or a cell described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

It is to be understood that both the foregoing general description and the following detailed description are exemplary and explanatory only and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### II. Definitions

Unless otherwise indicated, all technical terms used herein have the same meaning as commonly understood by one of ordinary skill in the art to which this invention belongs. Unless otherwise indicated or obvious from context, the following terms have the following meanings:

Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The term “effector protein” refers to a protein that is capable of modifying a nucleic acid molecule (e.g., by cleavage, deamination, recombination). Modifying the nucleic acid may modulate the expression of the nucleic acid molecule (e.g., increasing or decreasing the expression of a nucleic acid molecule). The effector protein may be a Cas protein (i.e., an effector protein of a CRISPR-Cas system). In some instances, an effector protein is a polypeptide that non-covalently binds to a guide nucleic acid to form a complex that contacts a target nucleic acid, wherein at least a portion of the guide nucleic acid hybridizes to a target sequence of the target nucleic acid. A complex between an effector protein and a guide nucleic acid can include multiple effector proteins or a single effector protein. In some instances, the effector protein modifies the target nucleic acid when the complex contacts the target nucleic acid. In some instances, the effector protein does not modify the target nucleic acid, but it is fused to a fusion partner protein that modifies the target nucleic acid when the complex contacts the target nucleic acid. A non-limiting example of an effector protein modifying a target nucleic acid is cleaving of a phosphodiester bond of the target nucleic acid. Additional examples of modifications an effector protein can make to target nucleic acids are described herein and throughout.

“Percent identity,” “% identity,” and % “identical” refers to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

As used in the specification and claims, the singular forms “a”, “an” and “the” include plural references unless the context clearly dictates otherwise. For example, the term “a sample” includes a plurality of samples, including mixtures thereof.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” refers to an animal. The subject may be a mammal. The subject may be a human. The subject may be diagnosed or at high risk for a disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

### III. Introduction

Disclosed herein are compositions, systems and methods comprising:
a) at least one of a polypeptide and a nucleic acid encoding the polypeptide; and
b) at least one of a guide nucleic acid and a DNA molecule encoding the guide nucleic acid.

Polypeptides described herein may bind and, optionally, cleave nucleic acids in a sequence-specific manner. Effector proteins described herein may bind a target sequence of a target nucleic acid and cleave the target nucleic acid within the target sequence or at a position adjacent to the target sequence. In some embodiments, a polypeptide is activated when it binds a target sequence of a target nucleic acid to cleave a region of the target nucleic acid that is near, but not adjacent to the target sequence. A polypeptide may be an effector protein, such as a CRISPR-associated (Cas) protein, which may be coupled to a guide nucleic acid that imparts activity or sequence selectivity to the polypeptide. In general, guide nucleic acids comprise a first nucleotide sequence for interaction with an effector protein; and a second nucleotide sequence that hybridizes to a target nucleic acid. In some instances, the first sequence may be non-covalently bound by an effector protein or hybridize to an additional nucleic acid, wherein the additional nucleic acid is non-covalently bound by the effector protein. The first sequence may be referred to herein as a repeat sequence or handle sequence. The second sequence may be referred to herein as a spacer sequence. A guide nucleic acid may be referred to interchangeable as a guide RNA, however it is understood that guide nucleic acids may comprise DNA, RNA, or a combination thereof (e.g., RNA with a thymine base). In some instances, guide nucleic acids may include a chemically modified nucleobase or phosphate backbone. The handle sequence, as used herein, refers to a sequence that binds non-covalently with an effector protein. In some instances, the handle sequence comprises all or a portion of a repeat sequence. In general, a single guide nucleic acid, also referred to as a single guide RNA (sgRNA), comprises a handle sequence. In some aspects, a handle sequence includes a portion of a tracrRNA sequence that is capable of being non-covalently bound by an effector protein, but does not include all or a part of the portion of a tracrRNA that hybridizes to a portion of a crRNA as found in a dual nucleic acid system. In some aspects, a handle sequence can include a portion of a tracrRNA sequence as well as a portion of a repeat sequence, which can optionally be connected by a linker. In some aspects, a handle sequence in the context of a sgRNA can also be described as the portion of the sgRNA that does not hybridize to a target sequence in a target nucleic acid (e.g., a spacer sequence).

Effector proteins disclosed herein may cleave nucleic acids, including single stranded RNA (ssRNA), double stranded DNA (dsDNA), and single-stranded DNA (ssDNA). Effector proteins disclosed herein may provide cis cleavage activity, trans cleavage activity, nickase activity, or a combination thereof.

The compositions, systems and methods described herein are non-naturally occurring. In some instances, compositions, systems and methods comprise an engineered guide nucleic acid or a use thereof. In some instances, compositions, systems and methods comprise an engineered polypeptide or a use thereof. In general, compositions and systems described herein are not found in nature. In some embodiments, compositions, methods and systems described herein comprise at least one non-naturally occurring component. For example, disclosed compositions, methods and systems may comprise a guide nucleic acid, wherein the sequence of the guide nucleic acid is different or modified from that of a naturally-occurring guide nucleic acid. In some embodiments, compositions, systems and methods comprise at least two components that do not naturally occur together. For example, disclosed compositions, methods and systems may comprise a guide nucleic acid comprising a repeat region and a spacer region which do not naturally occur together. Also, by way of non-limiting example, disclosed compositions, methods and systems may comprise a guide nucleic acid and an effector protein that do not naturally occur together. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some instances, the effector proteins are not found in nature. In some instances, the guide nucleic acids are not found in nature. In some instances, systems and compositions comprise at least one non-naturally occurring component. For example, compositions and systems may comprise a guide nucleic acid, wherein the sequence of the guide nucleic acid is different or modified from that of a naturally-occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, composition and systems may comprise a guide nucleic acid and an effector protein that do not naturally occur together. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some instances, the guide nucleic acid comprises a non-natural nucleobase sequence. In some instances, the non-natural sequence is a nucleobase sequence that is not found in nature. The non-natural sequence may comprise a portion of a naturally-occurring sequence, wherein the portion of the naturally-occurring sequence is not present in nature, absent the remainder of the naturally-occurring sequence. In some instances, the guide nucleic acid comprises two naturally-occurring sequences arranged in an order or proximity that is not observed in nature. In some instances, compositions and systems comprise a ribonucleotide complex comprising an effector protein and a guide nucleic acid that do not occur together in nature. Engineered guide nucleic acids may comprise a first sequence and a second sequence that do not occur naturally together. For example, an engineered guide nucleic acid may comprise a sequence of a naturally-occurring repeat region and a spacer region that is complementary to a naturally-occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence located at a 3′ or 5′ end of the guide nucleic acid, or between the first and second sequences of the guide nucleic acid. For example, an engineered guide nucleic acid may comprise a naturally occurring CRISPR RNA (crRNA) and trans-activating crRNA (tracrRNA) coupled by a linker sequence.

In some instances, compositions and systems described herein comprise an effector protein that is similar to a naturally occurring effector protein. The effector protein may lack a portion of the naturally occurring effector protein. The effector protein may comprise at least one amino acid substitution, deletion, or insertion relative to a naturally-occurring effector protein. In some instances, effector proteins comprise at least one additional amino acid relative to a naturally-occurring effector protein. For example, the effector protein may comprise a nuclear localization signal relative to the natural occurring effector protein. In certain embodiments, the nucleobase sequence encoding the effector protein is codon optimized (e.g., for expression in a eukaryotic cell) relative to a naturally occurring nucleobase sequence.

### IV. Effector Proteins

Provided herein, are compositions, systems, and methods comprising an effector protein or a use thereof.

An effector protein may be brought into proximity of a target nucleic acid in the presence of a guide nucleic acid when the guide nucleic acid includes a nucleotide sequence that is complementary with a target sequence in the target nucleic acid. The ability of an effector protein to modify a target nucleic acid may be dependent upon the effector protein being bound to a guide nucleic acid and the guide nucleic acid being hybridized to a target nucleic acid. An effector protein may also recognize a protospacer adjacent motif (PAM) sequence present in the target nucleic acid, which may direct the modification activity of the effector protein. In general, effector proteins described herein modify a target nucleic acid via cis cleavage activity on the target nucleic acid. The modification of the target nucleic acid generated by an effector protein may, as a non-limiting example, result in modulation of the expression of the target nucleic acid (e.g., increasing or decreasing expression of the nucleic acid) or modulation of the activity of a translation product of the target nucleic acid (e.g., inactivation of a protein binding to an RNA molecule or hybridization).

An effector protein may be a CRISPR-associated (Cas) protein. An effector protein may function as a single protein, including a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., dimer or multimer). An effector protein, when functioning in a multiprotein complex, may have only one functional activity (e.g., binding to a guide nucleic acid), while other effector proteins present in the multiprotein complex are capable of the other functional activity (e.g., modifying a target nucleic acid). An effector protein may be a modified effector protein having increased modification activity and/or increased substrate binding activity (e.g., substrate selectivity, specificity, and/or affinity). Alternatively, or in addition, an effector protein may be a catalytically inactive effector protein having reduced modification activity or no modification activity. Accordingly, an effector protein as used herein encompasses a modified polypeptide that does not have nuclease activity.

In certain embodiments, effector proteins described herein comprise one or more functional domains. Effector protein functional domains can include a protospacer adjacent motif (PAM)-interacting domain, an oligonucleotide-interacting domain, one or more recognition domains, a non-target strand interacting domain, and a RuvC domain. A PAM interacting domain can be a target strand PAM interacting domain (TPID) or a non-target strand PAM interacting domain (NTPID). In some embodiments, a PAM interacting domain, such as a TPID or a NTPID, on an effector protein describes a region of an effector protein that interacts with target nucleic acid. In some embodiments, the effector proteins comprise a RuvC domain. In some embodiments, a RuvC domain comprises with substrate binding activity, catalytic activity, or both. In some embodiments, the RuvC domain may be defined by a single, contiguous sequence, or a set of RuvC subdomains that are not contiguous with respect to the primary amino acid sequence of the protein. An effector protein of the present disclosure may include multiple RuvC subdomains, which may combine to generate a RuvC domain with substrate binding or catalytic activity. For example, an effector protein may include three RuvC subdomains (RuvC-I, RuvC-II, and RuvC-III) that are not contiguous with respect to the primary amino acid sequence of the effector protein, but form a RuvC domain once the protein is produced and folds. In some embodiments, effector proteins comprise one or more recognition domain (REC domain) with a binding affinity for a guide nucleic acid or for a guide nucleic acid-target nucleic acid heteroduplex. An effector protein may comprise a zinc finger domain. In some embodiments, the effector protein does not comprise an HNH domain.

An effector protein may be small, which may be beneficial for nucleic acid detection or editing (for example, the effector protein may be less likely to adsorb to a surface or another biological species due to its small size). The smaller nature of these effector proteins may allow for them to be more easily packaged and delivered with higher efficiency in the context of genome editing and more readily incorporated as a reagent in an assay.

Provided herein are compositions that comprise one or more effector proteins, as well as uses thereof. In some instances, the compositions are not naturally occurring. In some instances, the compositions comprises an engineered guide nucleic acid. In some instances, the nucleobase sequence of the engineered guide nucleic acid is not identical to the nucleobase sequence of a naturally occurring nucleic acid. In some instances, the nucleobase sequence of the engineered guide nucleic acid is less than 100%, less than 99%, less than 98%, less than 97%, less than 95%, less than 90%, less than 85%, less than 80%, or less than 75% identical to the nucleobase sequence of a naturally occurring nucleic acid

In some instances, effector proteins comprise an amino acid sequence selected from any one of SEQ ID NOS: 1-1614 and 3195-3302. In some instances, effector proteins consist essentially of an amino acid sequence selected from any one of SEQ ID NOS: 1-1614 and 3195-3302. In some instances, effector proteins consist of an amino acid sequence selected from any one of SEQ ID NOS: 1-1614 and 3195-3302. In some instances, effector proteins comprise an amino acid sequence that is at least 65%, identical to a sequence selected from any one of SEQ ID NOS: 1-1614 and 3195-3302. In some instances, effector proteins consist of an amino acid sequence that is at least 65% identical to a sequence selected from any one of SEQ ID NOS: 1-1614 and 3195-3302. In some instances, effector proteins comprise an amino acid sequence that is at least 70%, identical to a sequence selected from any one of SEQ ID NOS: 1-1614 and 3195-3302. In some instances, effector proteins consist of an amino acid sequence that is at least 70% identical to a sequence selected from any one of SEQ ID NOS: 1-1614 and 3195-3302. In some instances, effector proteins comprise an amino acid sequence that is at least 75%, identical to a sequence selected from any one of SEQ ID NOS: 1-1614 and 3195-3302. In some instances, effector proteins consist of an amino acid sequence that is at least 75% identical to a sequence selected from any one of SEQ ID NOS: 1-1614 and 3195-3302. In some instances, effector proteins comprise an amino acid sequence that is at least 80%, identical to a sequence selected from any one of SEQ ID NOS: 1-1614 and 3195-3302. In some instances, effector proteins consist of an amino acid sequence that is at least 80% identical to a sequence selected from any one of SEQ ID NOS: 1-1614 and 3195-3302. In some instances, effector proteins comprise an amino acid sequence that is at least 85%, identical to a sequence selected from any one of SEQ ID NOS: 1-1614 and 3195-3302. In some instances, effector proteins consist of an amino acid sequence that is at least 85% identical to a sequence selected from any one of SEQ ID NOS: 1-1614 and 3195-3302. In some instances, effector proteins comprise an amino acid sequence that is at least 90%, identical to a sequence selected from any one of SEQ ID NOS: 1-1614 and 3195-3302. In some instances, effector proteins consist of an amino acid sequence that is at least 90% identical to a sequence selected from any one of SEQ ID NOS: 1-1614 and 3195-3302. In some instances, effector proteins comprise an amino acid sequence that is at least 95%, identical to a sequence selected from any one of SEQ ID NOS: 1-1614 and 3195-3302. In some instances, effector proteins consist of an amino acid sequence that is at least 95% identical to a sequence selected from any one of SEQ ID NOS: 1-1614 and 3195-3302. In some instances, effector proteins comprise an amino acid sequence that is at least 97%, identical to a sequence selected from any one of SEQ ID NOS: 1-1614 and 3195-3302. In some instances, effector proteins consist of an amino acid sequence that is at least 97% identical to a sequence selected from any one of SEQ ID NOS: 1-1614 and 3195-3302. In some instances, effector proteins comprise an amino acid sequence that is at least 98%, identical to a sequence selected from any one of SEQ ID NOS: 1-1614 and 3195-3302. In some instances, effector proteins consist of an amino acid sequence that is at least 98% identical to a sequence selected from any one of SEQ ID NOS: 1-1614 and 3195-3302. In some instances, effector proteins comprise an amino acid sequence that is at least 99%, identical to a sequence selected from any one of SEQ ID NOS: 1-1614 and 3195-3302. In some instances, effector proteins consist of an amino acid sequence that is at least 99% identical to a sequence selected from any one of SEQ ID NOS: 1-1614 and 3195-3302. In some instances, effector proteins comprise an amino acid sequence that is 100%, identical to a sequence selected from any one of SEQ ID NOS: 1-1614 and 3195-3302. In some instances, effector proteins consist of an amino acid sequence that is 100% identical to a sequence selected from any one of SEQ ID NOS: 1-1614 and 3195-3302.

In some instances, effector proteins comprise at least about 100, at least about 120, at least about 140, at least about 160, at least about 180, at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500, at least about 520, at least about 540, at least about 560, at least about 580, at least about 600, at least about 620, at least about 640, at least about 660, at least about 680, at least about 700, at least about 720, at least about 740, at least about 760, at least about 780, at least about 800, at least about 820, at least about 840, at least about 860, at least about 880, at least about 900, at least about 920, at least about 940, at least about 960, at least about 980, at least about 1000, at least about 1020, at least about 1040, at least about 1060, at least about 1080, at least about 1100, at least about 1120, at least about 1140, at least about 1160, at least about 1180, or at least about 1200 contiguous amino acids of a sequence selected from any one of SEQ ID NOS: 1-1614 and 3195-3302. In some instances, effector proteins comprise less than about 1900, less than about 1850, less than about 1800, less than about 1750, less than about 1700, or less than about 1650 contiguous amino acids of a sequence selected from any one of SEQ ID NOS: 1-1614 and 3195-3302.

In some instances, effector proteins comprise about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, about 840, about 860, about 880, about 900, about 920, about 940, about 960, about 980, about 1000, about 1020, about 1040, about 1060, about 1080, about 1100, about 1120, about 1140, about 1160, about 1180, about 1200, about 1220, about 1240, about 1260, about 1280, about 1300, about 1320, about 1340, about 1360, about 1380, or about 1400 contiguous amino acids of a sequence selected from any one of SEQ ID NOS: 1-1614 and 3195-3302.

In some instances, compositions comprise an engineered guide nucleic acid (also referred to simply as a guide nucleic acid), wherein the guide nucleic acid comprises a nucleobase sequence selected from any one of SEQ ID NOS: 1615-3194 and 3303-3406. In some instances, guide nucleic acids comprise a sequence that is complementary to a nucleobase sequence selected from any one of SEQ ID NOS: 1615-3194 and 3303-3406. In some instances, guide nucleic acids comprise a sequence that is reverse complementary to a nucleobase sequence selected from any one of SEQ ID NOS: 1615-3194 and 3303-3406. In some instances, guide nucleic acids comprise a sequence that is at least 65% identical to a nucleobase sequence selected from any one of SEQ ID NOS: 1615-3194 and 3303-3406, the complement thereof, or the reverse complement thereof. In some instances, guide nucleic acids comprise a sequence that is at least 70% identical to a nucleobase sequence selected from any one of SEQ ID NOS: 1615-3194 and 3303-3406, the complement thereof, or the reverse complement thereof. In some instances, guide nucleic acids comprise a sequence that is at least 75% identical to a nucleobase sequence selected from any one of SEQ ID NOS: 1615-3194 and 3303-3406, the complement thereof, or the reverse complement thereof. In some instances, guide nucleic acids comprise a sequence that is at least 80% identical to a nucleobase sequence selected from any one of SEQ ID NOS: 1615-3194 and 3303-3406, the complement thereof, or the reverse complement thereof. In some instances, guide nucleic acids comprise a sequence that is at least 85% identical to a nucleobase sequence selected from any one of SEQ ID NOS: 1615-3194 and 3303-3406, the complement thereof, or the reverse complement thereof. In some instances, guide nucleic acids comprise a sequence that is at least 90% identical to a nucleobase sequence selected from any one of SEQ ID NOS: 1615-3194 and 3303-3406, the complement thereof, or the reverse complement thereof. In some instances, guide nucleic acids comprise a sequence that is 100% identical to a nucleobase sequence selected from any one of SEQ ID NOS: 1615-3194 and 3303-3406, the complement thereof, or the reverse complement thereof.

In some instances, guide nucleic acids comprise at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39 or at least 40 contiguous nucleotides of a nucleobase sequence selected from any one of SEQ ID NOS: 1615-3194 and 3303-3406, the complement thereof, or the reverse complement thereof. In some instances, guide nucleic acids contain less than 32, less than 34, less than 36, less than 37, less than 38, less than 39, less than 40, less than 41, less than 42, less than 43, less than 44, or less than 45 contiguous nucleotides of any one of the nucleobase sequences selected from any one of SEQ ID NOS: 1615-3194 and 3303-3406, the complement thereof, or the reverse complement thereof. In some instances, guide nucleic acids comprise 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 contiguous nucleotides of any one of the nucleobase sequences selected from any one of SEQ ID NOS: 1615-3194 and 3303-3406, the complement thereof, or the reverse complement thereof.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A1 of TABLE 1, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A2 of TABLE 1, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A3 of TABLE 1, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column C1 of TABLE 2, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column D1 of TABLE 2, wherein the sequence from Column C1 and the sequence from Column D1 are in the same row of TABLE 2.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column C2 of TABLE 2, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column D2 of TABLE 2, wherein the sequence from Column C2 and the sequence from Column D2 are in the same row of TABLE 2.

In some instances, compositions comprise an effector protein or a fusion protein thereof, and a guide nucleic acid, wherein the amino acid sequence of the effector protein is at least about 50% identical to a sequence selected from Column A1 of TABLE 1, and wherein at least a portion of the guide nucleic acid comprises a nucleobase sequence that is at least about 50% identical or at least about 50% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least about 60% identical to a sequence selected from Column A1 of TABLE 1, and wherein at least a portion of the guide nucleic acid comprises a nucleobase sequence that is at least about 60% identical or at least about 60% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least about 70% identical to a sequence selected from Column A1 of TABLE 1, and wherein at least a portion of the guide nucleic acid comprises a nucleobase sequence that is at least about 70% identical or at least about 70% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least about 80% identical to a sequence selected from Column A1 of TABLE 1, and wherein at least a portion of the guide nucleic acid comprises a nucleobase sequence that is at least about or 80% identical or at least about 80% reverse complementary to a sequence selected from Column B1 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 90% identical to a sequence selected from Column A1 of TABLE 1, and wherein at least a portion of the guide nucleic acid comprises a nucleobase sequence that is at least about 90% identical or at least about 90% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least about 95% identical to a sequence selected from Column A1 of TABLE 1, and wherein at least a portion of the guide nucleic acid comprises a nucleobase sequence that is at least about 95% identical or at least 95% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is 100% identical to a sequence selected from Column A1 of TABLE 1, and wherein at least a portion of the guide nucleic acid comprises a nucleobase sequence that is 100% identical or 100% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least about 50% identical to a sequence selected from Column A2 of TABLE 1, and wherein at least a portion of the guide nucleic acid comprises a nucleobase sequence that is at least about 50% identical or at least about 50% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 60% identical to a sequence selected from Column A2 of TABLE 1, and wherein at least a portion of the guide nucleic acid comprises a nucleobase sequence that is at least about 60% identical or at least about 60% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 70% identical to a sequence selected from Column A2 of TABLE 1, and wherein at least a portion of the guide nucleic acid comprises a nucleobase sequence that is at least about 70% identical or at least about 70% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 80% identical to a sequence selected from Column A2 of TABLE 1, and wherein at least a portion of the guide nucleic acid comprises a nucleobase sequence that is at least about 80% identical or at least about 80% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 90% identical to a sequence selected from Column A2 of TABLE 1, and wherein at least a portion of the guide nucleic acid comprises a nucleobase sequence that is at least about 90% identical or at least about 90% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 95% identical to a sequence selected from Column A2 of TABLE 1, and wherein at least a portion of the guide nucleic acid comprises a nucleobase sequence that is at least about 95% identical or at least about 95% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is 100% identical to a sequence selected from Column A2 of TABLE 1, and wherein at least a portion of the guide nucleic acid comprises a nucleobase sequence that is 100% identical or 100% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 50% identical to a sequence selected from Column A3 of TABLE 1, and wherein at least a portion of the guide nucleic acid comprises a nucleobase sequence that is at least about 50% identical or at least about 50% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 60% identical to a sequence selected from Column A3 of TABLE 1, and wherein at least a portion of the guide nucleic acid comprises a nucleobase sequence that is at least about 60% identical or at least about 60% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 70% identical to a sequence selected from Column A3 of TABLE 1, and wherein at least a portion of the guide nucleic acid comprises a nucleobase sequence that is at least about 70% identical or at least about 70% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 80% identical to a sequence selected from Column A3 of TABLE 1, and wherein at least a portion of the guide nucleic acid comprises a nucleobase sequence that is at least about 80% identical or at least about 80% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 90% identical to a sequence selected from Column A3 of TABLE 1, and wherein at least a portion of the guide nucleic acid comprises a nucleobase sequence that is at least about 90% identical or at least about 90% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 95% identical to a sequence selected from Column A3 of TABLE 1, and wherein at least a portion of the guide nucleic acid comprises a nucleobase sequence that is at least about 95% identical or at least about 95% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is 100% identical to a sequence selected from Column A3 of TABLE 1, and wherein at least a portion of the guide nucleic acid comprises a nucleobase sequence that is 100% identical or 100% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 50% identical to a sequence selected from Column C1 of TABLE 2, and wherein at least a portion of the guide nucleic acid comprises a nucleobase sequence that is at least about 50% identical or at least about 50% reverse complementary to a sequence selected from Column D1 of TABLE 2, wherein the sequence from Column C1 and the sequence from Column D1 are in the same row of TABLE 2.

In some instances, compositions comprise an effector protein or a fusion protein thereof, and a guide nucleic acid, wherein the amino acid sequence of the effector protein is at least about 60% identical to a sequence selected from Column C1 of TABLE 2, and wherein at least a portion of the guide nucleic acid comprises a nucleobase sequence that is at least about 60% identical or at least about 60% reverse complementary to a sequence selected from Column D1 of TABLE 2, wherein the sequence from Column C1 and the sequence from Column D1 are in the same row of TABLE 2.

In some instances, compositions comprise an effector protein or a fusion protein thereof, and a guide nucleic acid, wherein the amino acid sequence of the effector protein is at least about 70% identical to a sequence selected from Column C1 of TABLE 2, and wherein at least a portion of the guide nucleic acid comprises a nucleobase sequence that is at least about 70% identical or at least about 70% reverse complementary to a sequence selected from Column D1 of TABLE 2, wherein the sequence from Column C1 and the sequence from Column D1 are in the same row of TABLE 2.

In some instances, compositions comprise an effector protein or a fusion protein thereof, and a guide nucleic acid, wherein the amino acid sequence of the effector protein is at least about 80% identical to a sequence selected from Column C1 of TABLE 2, and wherein at least a portion of the guide nucleic acid comprises a nucleobase sequence that is at least about 80% identical or at least about 80% reverse complementary to a sequence selected from Column D1 of TABLE 2, wherein the sequence from Column C1 and the sequence from Column D1 are in the same row of TABLE 2.

In some instances, compositions comprise an effector protein or a fusion protein thereof, and a guide nucleic acid, wherein the amino acid sequence of the effector protein is at least about 90% identical to a sequence selected from Column C1 of TABLE 2, and wherein at least a portion of the guide nucleic acid comprises a nucleobase sequence that is at least about 90% identical or at least about 90% reverse complementary to a sequence selected from Column D1 of TABLE 2, wherein the sequence from Column C1 and the sequence from Column D1 are in the same row of TABLE 2.

In some instances, compositions comprise an effector protein or a fusion protein thereof, and a guide nucleic acid, wherein the amino acid sequence of the effector protein is at least about 95% identical to a sequence selected from Column C1 of TABLE 2, and wherein at least a portion of the guide nucleic acid comprises a nucleobase sequence that is at least about 95% identical or at least about 95% reverse complementary to a sequence selected from Column D1 of TABLE 2, wherein the sequence from Column C1 and the sequence from Column D1 are in the same row of TABLE 2.

In some instances, compositions comprise an effector protein or a fusion protein thereof, and a guide nucleic acid, wherein the amino acid sequence of the effector protein is 100% identical to a sequence selected from Column C1 of TABLE 2, and wherein at least a portion of the guide nucleic acid comprises a nucleobase sequence that is 100% identical or 100% reverse complementary to a sequence selected from Column D1 of TABLE 2, wherein the sequence from Column C1 and the sequence from Column D1 are in the same row of TABLE 2.

In some instances, compositions comprise an effector protein or a fusion protein thereof, and a guide nucleic acid, wherein the amino acid sequence of the effector protein is at least about 50% identical to a sequence selected from Column C2 of TABLE 2, and wherein at least a portion of the guide nucleic acid comprises a nucleobase sequence that is at least about 50% identical or at least about 50% reverse complementary to a sequence selected from Column D2 of TABLE 2, wherein the sequence from Column C2 and the sequence from Column D2 are in the same row of TABLE 2.

In some instances, compositions comprise an effector protein or a fusion protein thereof, and a guide nucleic acid, wherein the amino acid sequence of the effector protein is at least about 60% identical to a sequence selected from Column C2 of TABLE 2, and wherein at least a portion of the guide nucleic acid comprises a nucleobase sequence that is at least about 60% identical or at least about 60% reverse complementary to a sequence selected from Column D2 of TABLE 2, wherein the sequence from Column C2 and the sequence from Column D2 are in the same row of TABLE 2.

In some instances, compositions comprise an effector protein or a fusion protein thereof, and a guide nucleic acid, wherein the amino acid sequence of the effector protein is at least about 70% identical to a sequence selected from Column C2 of TABLE 2, and wherein at least a portion of the guide nucleic acid comprises a nucleobase sequence that is at least about 70% identical or at least about 70% reverse complementary to a sequence selected from Column D2 of TABLE 2, wherein the sequence from Column C2 and the sequence from Column D2 are in the same row of TABLE 2.

In some instances, compositions comprise an effector protein or a fusion protein thereof, and a guide nucleic acid, wherein the amino acid sequence of the effector protein is at least about 80% identical to a sequence selected from Column C2 of TABLE 2, and wherein at least a portion of the guide nucleic acid comprises a nucleobase sequence that is at least about 80% identical or at least about 80% reverse complementary to a sequence selected from Column D2 of TABLE 2, wherein the sequence from Column C2 and the sequence from Column D2 are in the same row of TABLE 2.

In some instances, compositions comprise an effector protein or a fusion protein thereof, and a guide nucleic acid, wherein the amino acid sequence of the effector protein is at least about 90% identical to a sequence selected from Column C2 of TABLE 2, and wherein at least a portion of the guide nucleic acid comprises a nucleobase sequence that is at least about 90% identical or at least about 90% reverse complementary to a sequence selected from Column D2 of TABLE 2, wherein the sequence from Column C2 and the sequence from Column D2 are in the same row of TABLE 2.

In some instances, compositions comprise an effector protein or a fusion protein thereof, and a guide nucleic acid, wherein the amino acid sequence of the effector protein is at least about 95% identical to a sequence selected from Column C2 of TABLE 2, and wherein at least a portion of the guide nucleic acid comprises a nucleobase sequence that is at least about 95% identical or at least about 95% reverse complementary to a sequence selected from Column D2 of TABLE 2, wherein the sequence from Column C2 and the sequence from Column D2 are in the same row of TABLE 2.

In some instances, compositions comprise an effector protein or a fusion protein thereof, and a guide nucleic acid, wherein the amino acid sequence of the effector protein is 100% identical to a sequence selected from Column C2 of TABLE 2, and wherein at least a portion of the guide nucleic acid comprises a nucleobase sequence that is 100% identical or 100% reverse complementary to a sequence selected from Column D2 of TABLE 2, wherein the sequence from Column C2 and the sequence from Column D2 are in the same row of TABLE 2.

In some embodiments, the effector protein comprises between 1-50 conservative substitutions relative to effector protein SEQ ID NOS: 1-1614 and 3195-3302. In some embodiments the effector protein comprises between 5-40 conservative substitutions relative to effector protein SEQ ID NOS: 1-1614 and 3195-3302. In some embodiments the effector protein comprises between 10-30 conservative substitutions relative to effector protein SEQ ID NOS: 1-1614 and 3195-3302. In some embodiments the effector protein comprises between 20-35 conservative substitutions relative to effector protein SEQ ID NOS: 1-1614 and 3195-3302. In general, conservative substitution refers to the replacement of one amino acid for another such that the replacement takes place within a family of amino acids that are related in their side chains. Genetically encoded amino acids can be divided into four families having related side chains: (1) acidic (negatively charged): Asp (D), Glu (G); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser (S), Thr (T). Amino acids may be related by aliphatic side chains: Gly (G), Ala (A), Val (V), Leu (L), Ile (I), Ser (S), Thr (T), with Ser (S) and Thr (T) optionally being grouped separately as aliphatic-hydroxyl; Amino acids may be related by aromatic side chains: Phe (F), Tyr (Y), Trp (W). Amino acids may be related by amide side chains: Asn (N), Glu (Q). Amino acids may be related by sulfur-containing side chains: Cys (C) and Met (M).

In some embodiments the effector protein comprises one conservative substitution relative to effector protein SEQ ID NOS: 1-1614 and 3195-3302. In some embodiments the effector protein comprises two conservative substitutions relative to effector protein SEQ ID NOS: 1-1614 and 3195-3302. In some embodiments the effector protein comprises three conservative substitutions relative to effector protein SEQ ID NOS: 1-1614 and 3195-3302. In some embodiments the effector protein comprises four conservative substitutions relative to effector protein SEQ ID NOS: 1-1614 and 3195-3302. In some embodiments the effector protein comprises five conservative substitutions relative to effector protein SEQ ID NOS: 1-1614 and 3195-3302. In some embodiments the effector protein comprises six conservative substitutions relative to effector protein SEQ ID NOS: 1-1614 and 3195-3302. In some embodiments the effector protein comprises seven conservative substitutions relative to effector protein SEQ ID NOS: 1-1614 and 3195-3302. In some embodiments the effector protein comprises eight conservative substitutions relative to effector protein SEQ ID NOS: 1-1614 and 3195-3302. In some embodiments the effector protein comprises nine conservative substitutions relative to effector protein SEQ ID NOS: 1-1614 and 3195-3302. In some embodiments the effector protein comprises ten conservative substitutions relative to effector protein SEQ ID NOS: 1-1614 and 3195-3302.

In some instances, the portion of the guide nucleic acid is the repeat region of the guide nucleic acid. In some instances, the portion of the guide nucleic acid binds the effector protein.

TABLE 1
Exemplary Effector Proteins and Guide Sequences

| A1 | B1 | A2 | B2 | A3 | B3 |
|---|---|---|---|---|---|
| SEQ | SEQ | SEQ | SEQ | SEQ | SEQ |
| ID NO: | ID NO: | ID NO: | ID NO: | ID NO: | ID NO: |
| 1 | 1615 | 539 | 2135 | 1077 | 2665 |
| 2 | 1616 | 540 | 2136 | 1078 | 2666 |
| 3 | 1617 | 541 | 2137 | 1079 | 2667 |
| 4 | 1618 | 542 | 2138 | 1080 | 2668 |
| 5 | 1619 | 543 | 2139 | 1081 | 2669 |
| 6 | 1620 | 544 | 2140 | 1082 | 2670 |
| 7 | 1621 | 545 | 2141 | 1083 | 2671 |
| 8 | 1622 | 546 | 2142 | 1084 | 2672 |
| 9 | 1623 | 547 | N/A | 1085 | 2673 |
| 10 | 1624 | 548 | 2143 | 1086 | 2674 |
| 11 | 1625 | 549 | 2144 | 1087 | 2675 |
| 12 | 1626 | 550 | 2145 | 1088 | 2676 |
| 13 | 1627 | 551 | 2146 | 1089 | 2677 |
| 14 | 1628 | 552 | 2147 | 1090 | 2678 |
| 15 | 1629 | 553 | 2148 | 1091 | 2679 |
| 16 | 1630 | 554 | 2149 | 1092 | 2680 |
| 17 | 1631 | 555 | 2150 | 1093 | 2681 |
| 18 | 1632 | 556 | 2151 | 1094 | 2682 |
| 19 | 1633 | 557 | 2152 | 1095 | 2683 |
| 20 | 1634 | 558 | 2153 | 1096 | 2684 |
| 21 | 1635 | 559 | 2154 | 1097 | 2685 |
| 22 | 1636 | 560 | 2155 | 1098 | 2686 |
| 23 | 1637 | 561 | 2156 | 1099 | 2687 |
| 24 | 1638 | 562 | 2157 | 1100 | 2688 |
| 25 | 1639 | 563 | 2158 | 1101 | 2689 |
| 26 | 1640 | 564 | 2159 | 1102 | 2690 |
| 27 | 1641 | 565 | 2160 | 1103 | 2691 |
| 28 | 1642 | 566 | 2161 | 1104 | 2692 |
| 29 | 1643 | 567 | 2162 | 1105 | 2693 |
| 30 | 1644 | 568 | 2163 | 1106 | 2694 |
| 31 | 1645 | 569 | 2164 | 1107 | 2695 |
| 32 | 1646 | 570 | 2165 | 1108 | 2696 |
| 33 | 1647 | 571 | 2166 | 1109 | 2697 |
| 34 | 1648 | 572 | 2167 | 1110 | 2698 |
| 35 | 1649 | 573 | 2168 | 1111 | 2699 |
| 36 | 1650 | 574 | 2169 | 1112 | 2700 |
| 37 | 1651 | 575 | 2170 | 1113 | 2701 |
| 38 | 1652 | 576 | 2171 | 1114 | 2702 |
| 39 | 1653 | 577 | 2172 | 1115 | 2703 |
| 40 | 1654 | 578 | 2173 | 1116 | 2704 |
| 41 | 1655 | 579 | 2174 | 1117 | 2705 |
| 42 | 1656 | 580 | 2175 | 1118 | 2706 |
| 43 | N/A | 581 | 2176 | 1119 | N/A |
| 44 | 1657 | 582 | 2177 | 1120 | 2707 |
| 45 | 1658 | 583 | 2178 | 1121 | 2708 |
| 46 | N/A | 584 | 2179 | 1122 | 2709 |
| 47 | 1659 | 585 | 2180 | 1123 | 2710 |
| 48 | 1660 | 586 | 2181 | 1124 | 2711 |
| 49 | 1661 | 587 | 2182 | 1125 | 2712 |
| 50 | 1662 | 588 | 2183 | 1126 | 2713 |
| 51 | 1663 | 589 | 2184 | 1127 | 2714 |
| 52 | 1664 | 590 | 2185 | 1128 | 2715 |
| 53 | 1665 | 591 | 2186 | 1129 | 2716 |
| 54 | 1666 | 592 | 2187 | 1130 | 2717 |
| 55 | 1667 | 593 | 2188 | 1131 | 2718 |
| 56 | 1668 | 594 | 2189 | 1132 | 2719 |
| 57 | 1669 | 595 | 2190 | 1133 | 2720 |
| 58 | 1670 | 596 | 2191 | 1134 | 2721 |
| 59 | 1671 | 597 | 2192 | 1135 | 2722 |
| 60 | 1672 | 598 | 2193 | 1136 | 2723 |
| 61 | 1673 | 599 | 2194 | 1137 | 2724 |
| 62 | 1674 | 600 | 2195 | 1138 | 2725 |
| 63 | 1675 | 601 | 2196 | 1139 | 2726 |
| 64 | 1676 | 602 | 2197 | 1140 | 2727 |
| 65 | 1677 | 603 | 2198 | 1141 | 2728 |
| 66 | 1678 | 604 | 2199 | 1142 | 2729 |
| 67 | 1679 | 605 | 2200 | 1143 | 2730 |
| 68 | 1680 | 606 | 2201 | 1144 | 2731 |
| 69 | 1681 | 607 | 2202 | 1145 | 2732 |
| 70 | 1682 | 608 | 2203 | 1146 | 2733 |
| 71 | 1683 | 609 | 2204 | 1147 | 2734 |
| 72 | 1684 | 610 | 2205 | 1148 | 2735 |
| 73 | 1685 | 611 | 2206 | 1149 | 2736 |
| 74 | 1686 | 612 | 2207 | 1150 | 2737 |
| 75 | 1687 | 613 | 2208 | 1151 | 2738 |
| 76 | 1688 | 614 | 2209 | 1152 | 2739 |
| 77 | 1689 | 615 | 2210 | 1153 | 2740 |
| 78 | 1690 | 616 | 2211 | 1154 | 2741 |
| 79 | 1691 | 617 | 2212 | 1155 | 2742 |
| 80 | 1692 | 618 | 2213 | 1156 | 2743 |
| 81 | 1693 | 619 | 2214 | 1157 | 2744 |
| 82 | 1694 | 620 | 2215 | 1158 | 2745 |
| 83 | 1695 | 621 | 2216 | 1159 | 2746 |
| 84 | 1696 | 622 | 2217 | 1160 | 2747 |
| 85 | 1697 | 623 | 2218 | 1161 | 2748 |
| 86 | 1698 | 624 | 2219 | 1162 | 2749 |
| 87 | 1699 | 625 | 2220 | 1163 | 2750 |
| 88 | 1700 | 626 | 2221 | 1164 | 2751 |
| 89 | 1701 | 627 | 2222 | 1165 | 2752 |
| 90 | 1702 | 628 | 2223 | 1166 | 2753 |
| 91 | 1703 | 629 | 2224 | 1167 | 2754 |
| 92 | 1704 | 630 | 2225 | 1168 | N/A |
| 93 | 1705 | 631 | 2226 | 1169 | 2755 |
| 94 | 1706 | 632 | 2227 | 1170 | 2756 |
| 95 | 1707 | 633 | 2228 | 1171 | 2757 |
| 96 | 1708 | 634 | 2229 | 1172 | 2758 |
| 97 | 1709 | 635 | 2230 | 1173 | 2759 |
| 98 | 1710 | 636 | 2231 | 1174 | 2760 |
| 99 | 1711 | 637 | N/A | 1175 | 2761 |
| 100 | 1712 | 638 | 2232 | 1176 | 2762 |
| 101 | 1713 | 639 | 2233 | 1177 | 2763 |
| 102 | 1714 | 640 | 2234 | 1178 | 2764 |
| 103 | 1715 | 641 | 2235 | 1179 | 2765 |
| 104 | 1716 | 642 | N/A | 1180 | 2766 |
| 105 | 1717 | 643 | 2236 | 1181 | 2767 |
| 106 | 1718 | 644 | 2237 | 1182 | 2768 |
| 107 | 1719 | 645 | 2238 | 1183 | 2769 |
| 108 | N/A | 646 | 2239 | 1184 | 2770 |
| 109 | 1720 | 647 | 2240 | 1185 | 2771 |
| 110 | 1721 | 648 | 2241 | 1186 | 2772 |
| 111 | 1722 | 649 | 2242 | 1187 | 2773 |
| 112 | 1723 | 650 | 2243 | 1188 | 2774 |
| 113 | 1724 | 651 | 2244 | 1189 | 2775 |
| 114 | 1725 | 652 | 2245 | 1190 | 2776 |
| 115 | 1726 | 653 | 2246 | 1191 | 2777 |
| 116 | 1727 | 654 | 2247 | 1192 | 2778 |
| 117 | 1728 | 655 | 2248 | 1193 | 2779 |
| 118 | 1729 | 656 | 2249 | 1194 | 2780 |
| 119 | 1730 | 657 | 2250 | 1195 | 2781 |
| 120 | 1731 | 658 | 2251 | 1196 | 2782 |
| 121 | 1732 | 659 | 2252 | 1197 | 2783 |
| 122 | 1733 | 660 | 2253 | 1198 | 2784 |
| 123 | 1734 | 661 | 2254 | 1199 | 2785 |
| 124 | 1735 | 662 | 2255 | 1200 | 2786 |
| 125 | 1736 | 663 | 2256 | 1201 | 2787 |
| 126 | 1737 | 664 | 2257 | 1202 | 2788 |
| 127 | 1738 | 665 | 2258 | 1203 | 2789 |
| 128 | 1739 | 666 | 2259 | 1204 | 2790 |
| 129 | 1740 | 667 | 2260 | 1205 | 2791 |
| 130 | 1741 | 668 | 2261 | 1206 | 2792 |
| 131 | 1742 | 669 | 2262 | 1207 | 2793 |
| 132 | 1743 | 670 | 2263 | 1208 | 2794 |
| 133 | 1744 | 671 | 2264 | 1209 | 2795 |
| 134 | 1745 | 672 | 2265 | 1210 | 2796 |
| 135 | 1746 | 673 | 2266 | 1211 | 2797 |
| 136 | 1747 | 674 | 2267 | 1212 | 2798 |
| 137 | 1748 | 675 | 2268 | 1213 | 2799 |
| 138 | 1749 | 676 | 2269 | 1214 | 2800 |
| 139 | 1750 | 677 | 2270 | 1215 | 2801 |
| 140 | 1751 | 678 | 2271 | 1216 | 2802 |
| 141 | 1752 | 679 | 2272 | 1217 | 2803 |
| 142 | 1753 | 680 | 2273 | 1218 | 2804 |
| 143 | 1754 | 681 | 2274 | 1219 | 2805 |
| 144 | 1755 | 682 | 2275 | 1220 | 2806 |
| 145 | 1756 | 683 | 2276 | 1221 | 2807 |
| 146 | 1757 | 684 | 2277 | 1222 | 2808 |
| 147 | N/A | 685 | 2278 | 1223 | 2809 |
| 148 | 1758 | 686 | 2279 | 1224 | 2810 |
| 149 | 1759 | 687 | 2280 | 1225 | 2811 |
| 150 | 1760 | 688 | 2281 | 1226 | 2812 |
| 151 | 1761 | 689 | 2282 | 1227 | 2813 |
| 152 | 1762 | 690 | 2283 | 1228 | 2814 |
| 153 | 1763 | 691 | 2284 | 1229 | 2815 |
| 154 | 1764 | 692 | 2285 | 1230 | 2816 |
| 155 | 1765 | 693 | 2286 | 1231 | 2817 |
| 156 | 1766 | 694 | 2287 | 1232 | 2818 |
| 157 | 1767 | 695 | 2288 | 1233 | 2819 |
| 158 | 1768 | 696 | 2289 | 1234 | 2820 |
| 159 | 1769 | 697 | N/A | 1235 | 2821 |
| 160 | 1770 | 698 | 2290 | 1236 | 2822 |
| 161 | 1771 | 699 | 2291 | 1237 | 2823 |
| 162 | 1772 | 700 | 2292 | 1238 | 2824 |
| 163 | N/A | 701 | 2293 | 1239 | 2825 |
| 164 | 1773 | 702 | N/A | 1240 | 2826 |
| 165 | 1774 | 703 | 2294 | 1241 | 2827 |
| 166 | 1775 | 704 | 2295 | 1242 | 2828 |
| 167 | 1776 | 705 | 2296 | 1243 | 2829 |
| 168 | 1777 | 706 | 2297 | 1244 | 2830 |
| 169 | 1778 | 707 | 2298 | 1245 | 2831 |
| 170 | 1779 | 708 | 2299 | 1246 | 2832 |
| 171 | 1780 | 709 | 2300 | 1247 | 2833 |
| 172 | 1781 | 710 | 2301 | 1248 | 2834 |
| 173 | 1782 | 711 | 2302 | 1249 | 2835 |
| 174 | 1783 | 712 | 2303 | 1250 | 2836 |
| 175 | 1784 | 713 | 2304 | 1251 | 2837 |
| 176 | 1785 | 714 | 2305 | 1252 | 2838 |
| 177 | 1786 | 715 | 2306 | 1253 | 2839 |
| 178 | 1787 | 716 | 2307 | 1254 | 2840 |
| 179 | 1788 | 717 | 2308 | 1255 | 2841 |
| 180 | N/A | 718 | 2309 | 1256 | 2842 |
| 181 | 1789 | 719 | 2310 | 1257 | 2843 |
| 182 | 1790 | 720 | 2311 | 1258 | 2844 |
| 183 | 1791 | 72 | 2312 | 1259 | 2845 |
| 184 | 1792 | 722 | 2313 | 1260 | 2846 |
| 185 | 1793 | 723 | 2314 | 1261 | 2847 |
| 186 | 1794 | 724 | 2315 | 1262 | 2848 |
| 187 | 1795 | 725 | 2316 | 1263 | 2849 |
| 188 | 1796 | 726 | 2317 | 1264 | 2850 |
| 189 | 1797 | 727 | 2318 | 1265 | 2851 |
| 190 | 1798 | 728 | 2319 | 1266 | 2852 |
| 191 | 1799 | 729 | 2320 | 1267 | 2853 |
| 192 | 1800 | 730 | 2321 | 1268 | 2854 |
| 193 | 1801 | 731 | 2322 | 1269 | 2855 |
| 194 | 1802 | 732 | 2323 | 1270 | 2856 |
| 195 | 1803 | 733 | 2324 | 1271 | 2857 |
| 196 | 1804 | 734 | 2325 | 1272 | 2858 |
| 197 | 1805 | 735 | 2326 | 1273 | 2859 |
| 198 | 1806 | 736 | 2327 | 1274 | N/A |
| 199 | 1807 | 737 | 2328 | 1275 | 2860 |
| 200 | 1808 | 738 | 2329 | 1276 | 2861 |
| 201 | 1809 | 739 | 2330 | 1277 | 2862 |
| 202 | 1810 | 740 | 2331 | 1278 | 2863 |
| 203 | 1811 | 741 | 2332 | 1279 | 2864 |
| 204 | 1812 | 742 | 2333 | 1280 | 2865 |
| 205 | N/A | 743 | 2334 | 1281 | 2866 |
| 206 | 1813 | 744 | 2335 | 1282 | 2867 |
| 207 | 1814 | 745 | 2336 | 1283 | 2868 |
| 208 | 1815 | 746 | 2337 | 1284 | 2869 |
| 209 | 1816 | 747 | 2338 | 1285 | 2870 |
| 210 | 1817 | 748 | 2339 | 1286 | 2871 |
| 211 | 1818 | 749 | 2340 | 1287 | 2872 |
| 212 | 1819 | 750 | 2341 | 1288 | 2873 |
| 213 | 1820 | 751 | 2342 | 1289 | 2874 |
| 214 | 1821 | 752 | N/A | 1290 | 2875 |
| 215 | 1822 | 753 | 2343 | 1291 | 2876 |
| 216 | 1823 | 754 | 2344 | 1292 | 2877 |
| 217 | 1824 | 755 | 2345 | 1293 | 2878 |
| 218 | 1825 | 756 | 2346 | 1294 | 2879 |
| 219 | 1826 | 757 | 2347 | 1295 | 2880 |
| 220 | 1827 | 758 | 2348 | 1296 | 2881 |
| 221 | 1828 | 759 | 2349 | 1297 | 2882 |
| 222 | 1829 | 760 | 2350 | 1298 | 2883 |
| 223 | 1830 | 761 | 2351 | 1299 | 2884 |
| 224 | 1831 | 762 | 2352 | 1300 | 2885 |
| 225 | 1832 | 763 | 2353 | 1301 | 2886 |
| 226 | 1833 | 764 | 2354 | 1302 | 2887 |
| 227 | 1834 | 765 | 2355 | 1303 | 2888 |
| 228 | 1835 | 766 | 2356 | 1304 | 2889 |
| 229 | 1836 | 767 | 2357 | 1305 | 2890 |
| 230 | 1837 | 768 | 2358 | 1306 | 2891 |
| 231 | 1838 | 769 | 2359 | 1307 | 2892 |
| 232 | 1839 | 770 | 2360 | 1308 | 2893 |
| 233 | 1840 | 771 | 2361 | 1309 | 2894 |
| 234 | 1841 | 772 | 2362 | 1310 | 2895 |
| 235 | 1842 | 773 | 2363 | 1311 | 2896 |
| 236 | 1843 | 774 | 2364 | 1312 | 2897 |
| 237 | 1844 | 775 | 2365 | 1313 | 2898 |
| 238 | 1845 | 776 | 2366 | 1314 | 2899 |
| 239 | 1846 | 777 | 2367 | 1315 | N/A |
| 240 | 1847 | 778 | 2368 | 1316 | N/A |
| 241 | 1848 | 779 | 2369 | 1317 | 2900 |
| 242 | 1849 | 780 | 2370 | 1318 | 2901 |
| 243 | 1850 | 781 | 2371 | 1319 | 2902 |
| 244 | 1851 | 782 | 2372 | 1320 | 2903 |
| 245 | 1852 | 783 | 2373 | 1321 | 2904 |
| 246 | 1853 | 784 | 2374 | 1322 | 2905 |
| 247 | 1854 | 785 | 2375 | 1323 | 2906 |
| 248 | 1855 | 786 | 2376 | 1324 | 2907 |
| 249 | 1856 | 787 | 2377 | 1325 | 2908 |
| 250 | 1857 | 788 | 2378 | 1326 | 2909 |
| 251 | 1858 | 789 | 2379 | 1327 | 2910 |
| 252 | 1859 | 790 | 2380 | 1328 | 2911 |
| 253 | 1860 | 791 | 2381 | 1329 | 2912 |
| 254 | 1861 | 792 | 2382 | 1330 | 2913 |
| 255 | 1862 | 793 | 2383 | 1331 | 2914 |
| 256 | 1863 | 794 | 2384 | 1332 | 2915 |
| 257 | 1864 | 795 | 2385 | 1333 | 2916 |
| 258 | 1865 | 796 | 2386 | 1334 | 2917 |
| 259 | 1866 | 797 | 2387 | 1335 | 2918 |
| 260 | 1867 | 798 | 2388 | 1336 | 2919 |
| 261 | 1868 | 799 | 2389 | 1337 | 2920 |
| 262 | 1869 | 800 | 2390 | 1338 | 2921 |
| 263 | 1870 | 801 | 2391 | 1339 | 2922 |
| 264 | 1871 | 802 | 2392 | 1340 | 2923 |
| 265 | 1872 | 803 | 2393 | 1341 | 2924 |
| 266 | 1873 | 804 | 2394 | 1342 | 2925 |
| 267 | 1874 | 805 | 2395 | 1343 | 2926 |
| 268 | 1875 | 806 | 2396 | 1344 | 2927 |
| 269 | 1876 | 807 | 2397 | 1345 | 2928 |
| 270 | 1877 | 808 | 2398 | 1346 | 2929 |
| 271 | 1878 | 809 | 2399 | 1347 | N/A |
| 272 | 1879 | 810 | 2400 | 1348 | 2930 |
| 273 | 1880 | 811 | 2401 | 1349 | 2931 |
| 274 | 1881 | 812 | 2402 | 1350 | 2932 |
| 275 | 1882 | 813 | 2403 | 1351 | 2933 |
| 276 | 1883 | 814 | 2404 | 1352 | 2934 |
| 277 | 1884 | 815 | 2405 | 1353 | 2935 |
| 278 | 1885 | 816 | 2406 | 1354 | 2936 |
| 279 | 1886 | 817 | 2407 | 1355 | 2937 |
| 280 | 1887 | 818 | 2408 | 1356 | 2938 |
| 281 | 1888 | 819 | 2409 | 1357 | 2939 |
| 282 | 1889 | 820 | 2410 | 1358 | 2940 |
| 283 | 1890 | 821 | 2411 | 1359 | 2941 |
| 284 | 1891 | 822 | 2412 | 1360 | 2942 |
| 285 | 1892 | 823 | 2413 | 1361 | 2943 |
| 286 | 1893 | 824 | 2414 | 1362 | 2944 |
| 287 | 1894 | 825 | 2415 | 1363 | 2945 |
| 288 | 1895 | 826 | 2416 | 1364 | 2946 |
| 289 | 1896 | 827 | 2417 | 1365 | 2947 |
| 290 | 1897 | 828 | 2418 | 1366 | 2948 |
| 291 | N/A | 829 | 2419 | 1367 | 2949 |
| 292 | 1898 | 830 | 2420 | 1368 | 2950 |
| 293 | 1899 | 831 | 2421 | 1369 | 2951 |
| 294 | 1900 | 832 | 2422 | 1370 | 2952 |
| 295 | 1901 | 833 | 2423 | 1371 | 2953 |
| 296 | 1902 | 834 | 2424 | 1372 | 2954 |
| 297 | 1903 | 835 | 2425 | 1373 | N/A |
| 298 | 1904 | 836 | 2426 | 1374 | 2955 |
| 299 | 1905 | 837 | 2427 | 1375 | 2956 |
| 300 | 1906 | 838 | 2428 | 1376 | 2957 |
| 301 | 1907 | 839 | 2429 | 1377 | 2958 |
| 302 | 1908 | 840 | 2430 | 1378 | 2959 |
| 303 | 1909 | 84 | 2431 | 1379 | 2960 |
| 304 | 1910 | 842 | 2432 | 1380 | 2961 |
| 305 | 1911 | 843 | 2433 | 1381 | 2962 |
| 306 | 1912 | 844 | 2434 | 1382 | 2963 |
| 307 | 1913 | 845 | 2435 | 1383 | 2964 |
| 308 | 1914 | 846 | 2436 | 1384 | 2965 |
| 309 | 1915 | 847 | 2437 | 1385 | 2966 |
| 310 | 1916 | 848 | 2438 | 1386 | 2967 |
| 311 | 1917 | 849 | 2439 | 1387 | 2968 |
| 312 | 1918 | 850 | 2440 | 1388 | 2969 |
| 313 | 1919 | 851 | 2441 | 1389 | 2970 |
| 314 | 1920 | 852 | N/A | 1390 | 2971 |
| 315 | 1921 | 853 | 2442 | 1391 | 2972 |
| 316 | 1922 | 854 | 2443 | 1392 | 2973 |
| 317 | 1923 | 855 | 2444 | 1393 | 2974 |
| 318 | 1924 | 856 | 2445 | 1394 | 2975 |
| 319 | 1925 | 857 | 2446 | 1395 | 2976 |
| 320 | 1926 | 858 | 2447 | 1396 | 2977 |
| 32 | 1927 | 859 | 2448 | 1397 | 2978 |
| 322 | 1928 | 860 | 2449 | 1398 | 2979 |
| 323 | 1929 | 861 | 2450 | 1399 | 2980 |
| 324 | 1930 | 862 | 2451 | 1400 | 2981 |
| 325 | 1931 | 863 | 2452 | 1401 | 2982 |
| 326 | 1932 | 864 | 2453 | 1402 | 2983 |
| 327 | 1933 | 865 | 2454 | 1403 | 2984 |
| 328 | 1934 | 866 | 2455 | 1404 | 2985 |
| 329 | 1935 | 867 | 2456 | 1405 | 2986 |
| 330 | 1936 | 868 | 2457 | 1406 | 2987 |
| 331 | 1937 | 869 | 2458 | 1407 | 2988 |
| 332 | 1938 | 870 | 2459 | 1408 | 2989 |
| 333 | N/A | 871 | 2460 | 1409 | 2990 |
| 334 | 1939 | 872 | 2461 | 1410 | 2991 |
| 335 | 1940 | 873 | 2462 | 1411 | 2992 |
| 336 | 1941 | 874 | 2463 | 1412 | 2993 |
| 337 | N/A | 875 | 2464 | 1413 | 2994 |
| 338 | 1942 | 876 | 2465 | 1414 | 2995 |
| 339 | N/A | 877 | 2466 | 1415 | 2996 |
| 340 | 1943 | 878 | 2467 | 1416 | 2997 |
| 341 | 1944 | 879 | 2468 | 1417 | 2998 |
| 342 | 1945 | 880 | 2469 | 1418 | 2999 |
| 343 | 1946 | 881 | 2470 | 1419 | 3000 |
| 344 | 1947 | 882 | 2471 | 1420 | 3001 |
| 345 | 1948 | 883 | 2472 | 1421 | 3002 |
| 346 | 1949 | 884 | 2473 | 1422 | 3003 |
| 347 | 1950 | 885 | 2474 | 1423 | 3004 |
| 348 | 1951 | 886 | 2475 | 1424 | 3005 |
| 349 | 1952 | 887 | 2476 | 1425 | 3006 |
| 350 | 1953 | 888 | 2477 | 1426 | 3007 |
| 351 | 1954 | 889 | 2478 | 1427 | 3008 |
| 352 | 1955 | 890 | 2479 | 1428 | 3009 |
| 353 | 1956 | 891 | 2480 | 1429 | 3010 |
| 354 | 1957 | 892 | 2481 | 1430 | 3011 |
| 355 | N/A | 893 | 2482 | 1431 | 3012 |
| 356 | 1958 | 894 | 2483 | 1432 | 3013 |
| 357 | 1959 | 895 | 2484 | 1433 | 3014 |
| 358 | 1960 | 896 | 2485 | 1434 | 3015 |
| 359 | 1961 | 897 | 2486 | 1435 | 3016 |
| 360 | 1962 | 898 | 2487 | 1436 | 3017 |
| 361 | 1963 | 899 | 2488 | 1437 | 3018 |
| 362 | 1964 | 900 | 2489 | 1438 | 3019 |
| 363 | 1965 | 901 | 2490 | 1439 | 3020 |
| 364 | 1966 | 902 | 2491 | 1440 | 3021 |
| 365 | 1967 | 903 | 2492 | 1441 | 3022 |
| 366 | 1968 | 904 | 2493 | 1442 | 3023 |
| 367 | 1969 | 905 | 2494 | 1443 | 3024 |
| 368 | 1970 | 906 | 2495 | 1444 | 3025 |
| 369 | 1971 | 907 | 2496 | 1445 | 3026 |
| 370 | 1972 | 908 | 2497 | 1446 | 3027 |
| 371 | 1973 | 909 | 2498 | 1447 | 3028 |
| 372 | 1974 | 910 | 2499 | 1448 | 3029 |
| 373 | 1975 | 911 | 2500 | 1449 | 3030 |
| 374 | 1976 | 912 | 2501 | 1450 | 3031 |
| 375 | 1977 | 913 | 2502 | 1451 | 3032 |
| 376 | N/A | 914 | 2503 | 1452 | 3033 |
| 377 | 1978 | 915 | 2504 | 1453 | 3034 |
| 378 | 1979 | 916 | 2505 | 1454 | 3035 |
| 379 | 1980 | 917 | 2506 | 1455 | 3036 |
| 380 | N/A | 918 | 2507 | 1456 | 3037 |
| 381 | 1981 | 919 | 2508 | 1457 | 3038 |
| 382 | 1982 | 920 | 2509 | 1458 | 3039 |
| 383 | 1983 | 921 | 2510 | 1459 | 3040 |
| 384 | 1984 | 922 | 2511 | 1460 | 3041 |
| 385 | 1985 | 923 | 2512 | 1461 | 3042 |
| 386 | 1986 | 924 | 2513 | 1462 | 3043 |
| 387 | 1987 | 925 | 2514 | 1463 | 3044 |
| 388 | 1988 | 926 | 2515 | 1464 | 3045 |
| 389 | 1989 | 927 | 2516 | 1465 | 3046 |
| 390 | 1990 | 928 | N/A | 1466 | 3047 |
| 391 | 1991 | 929 | 2517 | 1467 | 3048 |
| 392 | 1992 | 930 | 2518 | 1468 | 3049 |
| 393 | 1993 | 931 | 2519 | 1469 | 3050 |
| 394 | 1994 | 932 | 2520 | 1470 | 3051 |
| 395 | N/A | 933 | 2521 | 1471 | 3052 |
| 396 | 1995 | 934 | 2522 | 1472 | 3053 |
| 397 | 1996 | 935 | 2523 | 1473 | 3054 |
| 398 | 1997 | 936 | 2524 | 1474 | 3055 |
| 399 | 1998 | 937 | 2525 | 1475 | 3056 |
| 400 | 1999 | 938 | 2526 | 1476 | 3057 |
| 401 | 2000 | 939 | 2527 | 1477 | 3058 |
| 402 | 2001 | 940 | 2528 | 1478 | 3059 |
| 403 | 2002 | 941 | 2529 | 1479 | 3060 |
| 404 | 2003 | 942 | 2530 | 1480 | 3061 |
| 405 | 2004 | 943 | 2531 | 1481 | 3062 |
| 406 | 2005 | 944 | 2532 | 1482 | 3063 |
| 407 | 2006 | 945 | 2533 | 1483 | 3064 |
| 408 | 2007 | 946 | 2534 | 1484 | 3065 |
| 409 | 2008 | 947 | 2535 | 1485 | 3066 |
| 410 | 2009 | 948 | 2536 | 1486 | 3067 |
| 411 | 2010 | 949 | 2537 | 1487 | 3068 |
| 412 | 2011 | 950 | 2538 | 1488 | 3069 |
| 413 | 2012 | 951 | 2539 | 1489 | 3070 |
| 414 | 2013 | 952 | 2540 | 1490 | 3071 |
| 415 | 2014 | 953 | 2541 | 1491 | 3072 |
| 416 | 2015 | 954 | 2542 | 1492 | 3073 |
| 417 | 2016 | 955 | 2543 | 1493 | 3074 |
| 418 | 2017 | 956 | 2544 | 1494 | 3075 |
| 419 | 2018 | 957 | 2545 | 1495 | 3076 |
| 420 | 2019 | 958 | 2546 | 1496 | 3077 |
| 421 | 2020 | 959 | 2547 | 1497 | 3078 |
| 422 | 2021 | 960 | 2548 | 1498 | 3079 |
| 423 | 2022 | 961 | 2549 | 1499 | 3080 |
| 424 | 2023 | 962 | 2550 | 1500 | 3081 |
| 425 | 2024 | 963 | 2551 | 1501 | 3082 |
| 426 | 2025 | 964 | 2552 | 1502 | 3083 |
| 427 | 2026 | 965 | 2553 | 1503 | N/A |
| 428 | 2027 | 966 | 2554 | 1504 | 3084 |
| 429 | 2028 | 967 | 2555 | 1505 | 3085 |
| 430 | 2029 | 968 | 2556 | 1506 | 3086 |
| 431 | 2030 | 969 | 2557 | 1507 | 3087 |
| 432 | 2031 | 970 | 2558 | 1508 | 3088 |
| 433 | 2032 | 971 | 2559 | 1509 | 3089 |
| 434 | 2033 | 972 | 2560 | 1510 | 3090 |
| 435 | 2034 | 973 | 2561 | 1511 | 3091 |
| 436 | 2035 | 974 | 2562 | 1512 | 3092 |
| 437 | 2036 | 975 | 2563 | 1513 | 3093 |
| 438 | 2037 | 976 | 2564 | 1514 | 3094 |
| 439 | 2038 | 977 | 2565 | 1515 | 3095 |
| 440 | 2039 | 978 | 2566 | 1516 | 3096 |
| 441 | 2040 | 979 | 2567 | 1517 | 3097 |
| 442 | 2041 | 980 | 2568 | 1518 | 3098 |
| 443 | 2042 | 981 | 2569 | 1519 | 3099 |
| 444 | 2043 | 982 | 2570 | 1520 | 3100 |
| 445 | N/A | 983 | 2571 | 1521 | 3101 |
| 446 | 2044 | 984 | 2572 | 1522 | 3102 |
| 447 | 2045 | 985 | 2573 | 1523 | 3103 |
| 448 | 2046 | 986 | 2574 | 1524 | 3104 |
| 449 | 2047 | 987 | 2575 | 1525 | 3105 |
| 450 | 2048 | 988 | 2576 | 1526 | 3106 |
| 451 | 2049 | 989 | 2577 | 1527 | 3107 |
| 452 | 2050 | 990 | 2578 | 1528 | 3108 |
| 453 | 2051 | 991 | 2579 | 1529 | 3109 |
| 454 | 2052 | 992 | 2580 | 1530 | 3110 |
| 455 | 2053 | 993 | 2581 | 1531 | 3111 |
| 456 | 2054 | 994 | 2582 | 1532 | 3112 |
| 457 | 2055 | 995 | 2583 | 1533 | 3113 |
| 458 | 2056 | 996 | 2584 | 1534 | 3114 |
| 459 | 2057 | 997 | 2585 | 1535 | 3115 |
| 460 | 2058 | 998 | 2586 | 1536 | 3116 |
| 461 | 2059 | 999 | 2587 | 1537 | 3117 |
| 462 | 2060 | 1000 | 2588 | 1538 | 3118 |
| 463 | 2061 | 1001 | 2589 | 1539 | 3119 |
| 464 | 2062 | 1002 | 2590 | 1540 | 3120 |
| 465 | 2063 | 1003 | 2591 | 1541 | 3121 |
| 466 | 2064 | 1004 | 2592 | 1542 | 3122 |
| 467 | 2065 | 1005 | 2593 | 1543 | 3123 |
| 468 | 2066 | 1006 | 2594 | 1544 | 3124 |
| 469 | 2067 | 1007 | 2595 | 1545 | 3125 |
| 470 | 2068 | 1008 | 2596 | 1546 | 3126 |
| 471 | 2069 | 1009 | 2597 | 1547 | 3127 |
| 472 | 2070 | 1010 | 2598 | 1548 | 3128 |
| 473 | 2071 | 1011 | 2599 | 1549 | 3129 |
| 474 | 2072 | 1012 | 2600 | 1550 | 3130 |
| 475 | 2073 | 1013 | 2601 | 1551 | 3131 |
| 476 | 2074 | 1014 | 2602 | 1552 | 3132 |
| 477 | 2075 | 1015 | 2603 | 1553 | 3133 |
| 478 | 2076 | 1016 | 2604 | 1554 | 3134 |
| 479 | 2077 | 1017 | 2605 | 1555 | 3135 |
| 480 | 2078 | 1018 | 2606 | 1556 | 3136 |
| 481 | 2079 | 1019 | 2607 | 1557 | 3137 |
| 482 | 2080 | 1020 | 2608 | 1558 | 3138 |
| 483 | 2081 | 1021 | 2609 | 1559 | 3139 |
| 484 | 2082 | 1022 | 2610 | 1560 | 3140 |
| 485 | 2083 | 1023 | 2611 | 1561 | 3141 |
| 486 | 2084 | 1024 | 2612 | 1562 | 3142 |
| 487 | 2085 | 1025 | 2613 | 1563 | 3143 |
| 488 | 2086 | 1026 | 2614 | 1564 | 3144 |
| 489 | 2087 | 1027 | 2615 | 1565 | 3145 |
| 490 | 2088 | 1028 | 2616 | 1566 | 3146 |
| 491 | 2089 | 1029 | 2617 | 1567 | 3147 |
| 492 | 2090 | 1030 | 2618 | 1568 | 3148 |
| 493 | 2091 | 1031 | 2619 | 1569 | 3149 |
| 494 | 2092 | 1032 | 2620 | 1570 | 3150 |
| 495 | N/A | 1033 | 2621 | 1571 | 3151 |
| 496 | 2093 | 1034 | 2622 | 1572 | 3152 |
| 497 | 2094 | 1035 | 2623 | 1573 | 3153 |
| 498 | 2095 | 1036 | 2624 | 1574 | 3154 |
| 499 | 2096 | 1037 | 2625 | 1575 | 3155 |
| 500 | 2097 | 1038 | 2626 | 1576 | 3156 |
| 501 | 2098 | 1039 | 2627 | 1577 | 3157 |
| 502 | 2099 | 1040 | 2628 | 1578 | 3158 |
| 503 | 2100 | 1041 | 2629 | 1579 | 3159 |
| 504 | 2101 | 1042 | 2630 | 1580 | 3160 |
| 505 | 2102 | 1043 | 2631 | 1581 | 3161 |
| 506 | 2103 | 1044 | 2632 | 1582 | 3162 |
| 507 | 2104 | 1045 | 2633 | 1583 | 3163 |
| 508 | 2105 | 1046 | 2634 | 1584 | 3164 |
| 509 | 2106 | 1047 | 2635 | 1585 | 3165 |
| 510 | 2107 | 1048 | 2636 | 1586 | 3166 |
| 511 | 2108 | 1049 | 2637 | 1587 | 3167 |
| 512 | N/A | 1050 | 2638 | 1588 | 3168 |
| 513 | 2109 | 1051 | 2639 | 1589 | 3169 |
| 514 | 2110 | 1052 | 2640 | 1590 | 3170 |
| 515 | 2111 | 1053 | 2641 | 1591 | 3171 |
| 516 | 2112 | 1054 | 2642 | 1592 | 3172 |
| 517 | 2113 | 1055 | 2643 | 1593 | 3173 |
| 518 | 2114 | 1056 | 2644 | 1594 | 3174 |
| 519 | 2115 | 1057 | 2645 | 1595 | 3175 |
| 520 | 2116 | 1058 | 2646 | 1596 | 3176 |
| 521 | 2117 | 1059 | 2647 | 1597 | 3177 |
| 522 | 2118 | 1060 | 2648 | 1598 | 3178 |
| 523 | 2119 | 1061 | 2649 | 1599 | 3179 |
| 524 | 2120 | 1062 | 2650 | 1600 | 3180 |
| 525 | 2121 | 1063 | 2651 | 1601 | 3181 |
| 526 | 2122 | 1064 | 2652 | 1602 | 3182 |
| 527 | 2123 | 1065 | 2653 | 1603 | 3183 |
| 528 | 2124 | 1066 | 2654 | 1604 | 3184 |
| 529 | 2125 | 1067 | 2655 | 1605 | 3185 |
| 530 | 2126 | 1068 | 2656 | 1606 | 3186 |
| 531 | 2127 | 1069 | 2657 | 1607 | 3187 |
| 532 | 2128 | 1070 | 2658 | 1608 | 3188 |
| 533 | 2129 | 1071 | 2659 | 1609 | 3189 |
| 534 | 2130 | 1072 | 2660 | 1610 | 3190 |
| 535 | 2131 | 1073 | 2661 | 1611 | 3191 |
| 536 | 2132 | 1074 | 2662 | 1612 | 3192 |
| 537 | 2133 | 1075 | 2663 | 1613 | 3193 |
| 538 | 2134 | 1076 | 2664 | 1614 | 3194 |

TABLE 2
Exemplary Effector Proteins and Guide Sequences

| C1 | D1 | C2 | D2 |
|---|---|---|---|
| SEQ ID NO: | SEQ ID NO: | SEQ ID NO: | SEQ ID NO: |
| 3195 | N/A | 3249 | 3353 |
| 3196 | N/A | 3250 | 3354 |
| 3197 | N/A | 3251 | 3355 |
| 3198 | N/A | 3252 | 3356 |
| 3199 | 3303 | 3253 | 3357 |
| 3200 | 3304 | 3254 | 3358 |
| 3201 | 3305 | 3255 | 3359 |
| 3202 | 3306 | 3256 | 3360 |
| 3203 | 3307 | 3257 | 3361 |
| 3204 | 3308 | 3258 | 3362 |
| 3205 | 3309 | 3259 | 3363 |
| 3206 | 3310 | 3260 | 3364 |
| 3207 | 3311 | 3261 | 3365 |
| 3208 | 3312 | 3262 | 3366 |
| 3209 | 3313 | 3263 | 3367 |
| 3210 | 3314 | 3264 | 3368 |
| 3211 | 3315 | 3265 | 3369 |
| 3212 | 3316 | 3266 | 3370 |
| 3213 | 3317 | 3267 | 3371 |
| 3214 | 3318 | 3268 | 3372 |
| 3215 | 3319 | 3269 | 3373 |
| 3216 | 3320 | 3270 | 3374 |
| 3217 | 3321 | 3271 | 3375 |
| 3218 | 3322 | 3272 | 3376 |
| 3219 | 3323 | 3273 | 3377 |
| 3220 | 3324 | 3274 | 3378 |
| 3221 | 3325 | 3275 | 3379 |
| 3222 | 3326 | 3276 | 3380 |
| 3223 | 3327 | 3277 | 3381 |
| 3224 | 3328 | 3278 | 3382 |
| 3225 | 3329 | 3279 | 3383 |
| 3226 | 3330 | 3280 | 3384 |
| 3227 | 3331 | 3281 | 3385 |
| 3228 | 3332 | 3282 | 3386 |
| 3229 | 3333 | 3283 | 3387 |
| 3230 | 3334 | 3284 | 3388 |
| 3231 | 3335 | 3285 | 3389 |
| 3232 | 3336 | 3286 | 3390 |
| 3233 | 3337 | 3287 | 3391 |
| 3234 | 3338 | 3288 | 3392 |
| 3235 | 3339 | 3289 | 3393 |
| 3236 | 3340 | 3290 | 3394 |
| 3237 | 3341 | 3291 | 3395 |
| 3238 | 3342 | 3292 | 3396 |
| 3239 | 3343 | 3293 | 3397 |
| 3240 | 3344 | 3294 | 3398 |
| 3241 | 3345 | 3295 | 3399 |
| 3242 | 3346 | 3296 | 3400 |
| 3243 | 3347 | 3297 | 3401 |
| 3244 | 3348 | 3298 | 3402 |
| 3245 | 3349 | 3299 | 3403 |
| 3246 | 3350 | 3300 | 3404 |
| 3247 | 3351 | 3301 | 3405 |
| 3248 | 3352 | 3302 | 3406 |

In some cases, effector proteins comprise a RuvC domain. In some instances, a RuvC domain is a region of an effector protein that is capable of cleaving a target nucleic acid. In some instances, a RuvC domain is capable of processing a pre-crRNA. In some instances, the RuvC domain is located near the C-terminus of the effector protein. A single RuvC domain may comprise RuvC subdomains, for example, a RuvCI subdomain, a RuvCII subdomain, and a RuvCIII subdomain. The term “RuvC” domain may also be referred to as a “RuvC-like” domain. Various RuvC-like domains are known in the art and may be identified using online tools such as InterPro (https://www.ebi.ac.uk/interpro/). For example, a RuvC-like domain may be a domain which shares homology with a region of TnpB proteins of the IS605 and other related families of transposons.

In some instances, the length of the effector protein is at least 700 linked amino acid residues. In some instances, the length of the effector protein is less than 2000 linked amino acid residues. In some instances, the length of the effector protein is about 700 to about 800 linked amino acid residues. In some instances, the length of the effector protein is about 800 to about 900 linked amino acid residues. In some instances, the length of the effector protein is about 900 to about 1000 linked amino acid residues. In some instances, the length of the effector protein is about 1000 to about 1100 linked amino acid residues. In some instances, the length of the effector protein is about 1100 to about 1200 linked amino acid residues. In some instances, the length of the effector protein is about 1200 to about 1300 linked amino acid residues. In some instances, the length of the effector protein is about 1300 to about 1400 linked amino acid residues. In some instances, the length of the effector protein is about 1400 to about 1500 linked amino acid residues. In some instances, the length of the effector protein is about 1500 to about 1600 linked amino acid residues. In some instances, the length of the effector protein is about 1600 to about 1700 linked amino acid residues. In some instances, the length of the effector protein is about 1700 to about 1800 linked amino acid residues. In some instances, the length of the effector protein is about 1900 to about 2000 linked amino acid residues.

In some instances, the effector proteins function as an endonuclease that catalyzes cleavage within a target nucleic acid. In some instances, the effector proteins catalyze non-sequence-specific cleavage of a single stranded nucleic acid. In some instances, the effector proteins are activated to perform trans cleavage activity after binding of a guide nucleic acid with a target nucleic acid. This trans cleavage activity may also be referred to as “collateral” or “transcollateral” cleavage. Trans cleavage activity may be non-specific cleavage of nearby single-stranded nucleic acid by the activated effector protein, such as trans cleavage of detector nucleic acids with a detection moiety.

Effector proteins disclosed herein may catalyze cleavage at a specific position (e.g., at a specific nucleotide within a nucleic acid sequence) in a target nucleic acid. The target nucleic acid may be single stranded RNA (ssRNA), double stranded DNA (dsDNA) or single-stranded DNA (ssDNA). In some instances, the target nucleic acid is single-stranded DNA. In some instances, the target nucleic acid is single-stranded RNA. The effector proteins may provide cis cleavage activity, trans cleavage activity, nickase activity, or a combination thereof. Cis cleavage activity may comprise cleavage of a target nucleic acid that is hybridized to a guide nucleic acid, wherein cleavage occurs within or directly adjacent to the region of the target nucleic acid that is hybridized to the guide nucleic acid. Trans cleavage activity (also referred to as transcollateral cleavage) may comprise cleavage of ssDNA or ssRNA that is near, but not hybridized to the guide nucleic acid. Trans cleavage activity may be triggered by the hybridization of a guide nucleic acid to the target nucleic acid. Nickase activity may comprise a selective cleavage of one strand of a dsDNA.

Effector proteins of the present disclosure, dimers thereof, and multimeric complexes thereof may cleave or nick a target nucleic acid within or near a protospacer adjacent motif (PAM) sequence of the target nucleic acid. In general, the PAM is a nucleotide sequence found in a target nucleic acid that directs an effector protein to modify the target nucleic acid at a specific location. In some instances, cleavage occurs within 1, 2, 3, 4, 5, 6, 7, 8, 9 or 10 nucleosides of a 5′ or 3′ terminus of a PAM sequence. A target nucleic acid may comprise a PAM sequence adjacent to a sequence that is complementary to a guide nucleic acid spacer region.

### Engineered Proteins

In some embodiments, effector proteins described herein have been modified (also referred to as an engineered protein). In some instances, effector proteins disclosed herein are engineered proteins. Engineered proteins are not identical to a naturally-occurring protein.

For example, effector proteins described herein can be modified with the addition of one or more heterologous peptides or heterologous polypeptides. In some embodiments, heterologous peptide or heterologous polypeptide comprises a subcellular localization signal. In some embodiments, a subcellular localization signal can be a nuclear localization signal (NLS) for targeting the effector protein to the nucleus. In some embodiments, the subcellular localization signal is a nuclear export signal (NES), a sequence to keep a fusion protein retained in the cytoplasm, a mitochondrial localization signal for targeting to the mitochondria, a chloroplast localization signal for targeting to a chloroplast, an ER retention signal, and the like.

A subcellular localization signal may be located at or near the amino terminus (N-terminus) of the effector protein disclosed herein. Subcellular localization signal may be located at or near the carboxy terminus (C-terminus) of the effector protein is disclosed herein. In some embodiments, a vector encodes the effector proteins described herein, wherein the vector or vector systems disclosed herein comprises one or more subcellular localization signals, such as about or more than about 1, 2, 3, 4, 5, 6, 7, 8, 9, 10, or more subcellular localization signals. In some embodiments, an effector protein described herein comprises 1, 2, 3, 4, 5, 6, 7, 8, 9, 10, or more subcellular localization signals at or near the N-terminus, 1, 2, 3, 4, 5, 6, 7, 8, 9, 10, or more subcellular localization signals at or near the C-terminus, or a combination of these (e.g. one or more subcellular localization signals at the amino-terminus and one or more subcellular localization signals at the carboxy terminus). When more than one subcellular localization signal is present, each may be selected independently of the others, such that a single subcellular localization signal may be present in more than one copy and/or in combination with one or more other subcellular localization signals present in one or more copies. In some embodiments, a subcellular localization signal is considered near the N- or C-terminus when the nearest amino acid of the subcellular localization signal is within about 1, 2, 3, 4, 5, 10, 15, 20, 25, 30, 40, 50, or more amino acids along the polypeptide chain from the N- or C-terminus. In certain embodiments, a subcellular localization signal described herein comprises a subcellular localization signals sequence recited in Table. Accordingly, in some embodiments, effector proteins described herein comprise an amino acid sequence that at least about 65%, at least about 70%, at least about 75%, at least about 80%, at least about 85%, at least about 90%, at least about 95%, at least about 97%, or at least 98%, at least about 99%, or about 100% identical to any one of the sequences as selected from SEQ ID NOS: 1-1614 and 3195-3302 and further comprises one or more sequence set forth in Table. In some embodiments, an effector protein described herein is not modified with a subcellular localization signals so that the polypeptide is not targeted to the nucleus, which can be advantageous depending on the circumstance (e.g., when the target nucleic acid is an RNA that is present in the cytosol).

TABLE 3
SEQUENCES OF EFFECTOR PROTEIN MODIFICATIONS

| SEQ | | |
|---|---|---|
| ID | | |
| NO: | Description | Sequence* |
| 3407 | NLS | KR(K/R)R |
| 3408 | NLS | K(K/R)RK |
| 3409 | NLS | (P/R)XXKR(^DE)(K/R) |
| 3410 | NLS | KRX(W/F/Y)XXAF |
| 3411 | NLS | (R/P)XXKR(K/R)(^DE) |
| 3412 | NLS | LGKR(K/R)(W/F/Y) |
| 3413 | NLS | KRX₁₀₋₁₂K(K/R)(K/R) |
| 3414 | NLS | KRX₁₀₋₁₂K(K/R)X(K/R) |

*wherein X is any amino-acid; and ^D/E is any amino-acid except Asp or Glu

In some embodiments, effector proteins described herein can be modified with a protein tag. In some instances, the tag is referred to as purification tag or a fluorescent protein. A protein tag may comprise a peptide that is heterologous to the effector protein. The protein tag may be detectable for use in detection of the effector protein and/or purification of the effector protein. Accordingly, in some embodiments, compositions, systems and methods comprise a protein tag or use thereof. Non-limiting examples of protein tags include a fluorescent protein, a histidine tag, e.g., a 6×His tag; a hemagglutinin (HA) tag; a FLAG tag; a Myc tag; and maltose binding protein (MBP). In some instances, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry, and tdTomato.

In another example, effector proteins may be codon optimized. In some embodiments, effector proteins described herein are encoded by a codon optimized nucleic acid. In some embodiments, a nucleic acid sequence encoding an effector protein described herein, is codon optimized. This type of optimization can entail a mutation of an effector protein encoding nucleotide sequence to mimic the codon preferences of the intended host organism or cell while encoding the same polypeptide. Thus, the codons can be changed, but the encoded protein remains unchanged. For example, if the intended target cell was a human cell, a human codon-optimized effector protein-encoding nucleotide sequence could be used. As another non-limiting example, if the intended host cell were a mouse cell, then a mouse codon-optimized effector protein-encoding nucleotide sequence could be generated. As another non-limiting example, if the intended host cell were a eukaryotic cell, then a eukaryote codon-optimized effector protein nucleotide sequence could be generated. As another non-limiting example, if the intended host cell were a prokaryotic cell, then a prokaryote codon-optimized effector protein-encoding nucleotide sequence could be generated. Codon usage tables are readily available, for example, at the “Codon Usage Database” available at www.kazusa.or.jp/codon. Accordingly, in some embodiments, effector proteins described herein may be codon optimized for expression in a specific cell, for example, a bacterial cell, a plant cell, a eukaryotic cell, an animal cell, a mammalian cell, or a human cell. In some embodiments, the effector protein is codon optimized for a human cell.

It is understood that when describing coding sequences of polypeptides described herein, said coding sequences do not necessarily require a codon encoding a N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some embodiments, when a modifying heterologous peptide, such as a fusion partner protein, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

Engineered proteins may provide enhanced nuclease or nickase activity as compared to a naturally occurring nuclease or nickase. Engineered proteins may provide enhance nucleic acid binding activity, e.g., enhanced binding of a guide nucleic acid and/or target nucleic acid. By way of non-limiting example, some engineered proteins exhibit optimal activity at lower salinity and viscosity than the protoplasm of their bacterial cell of origin. Also, by way of non-limiting example, bacteria often comprise protoplasmic salt concentrations greater than 250 mM and room temperature intracellular viscosities above 2 centipoise, whereas engineered proteins exhibit optimal activity (e.g., cis-cleavage activity) at salt concentrations below 150 mM and viscosities below 1.5 centipoise. The present disclosure leverages these dependencies by providing engineered proteins in solutions optimized for their activity and stability.

Compositions and systems described herein may comprise an engineered effector protein in a solution comprising a room temperature viscosity of less than about 15 centipoise, less than about 12 centipoise, less than about 10 centipoise, less than about 8 centipoise, less than about 6 centipoise, less than about 5 centipoise, less than about 4 centipoise, less than about 3 centipoise, less than about 2 centipoise, or less than about 1.5 centipoise.

Compositions and systems may comprise an engineered effector protein in a solution comprising an ionic strength of less than about 500 mM, less than about 400 mM, less than about 300 mM, less than about 250 mM, less than about 200 mM, less than about 150 mM, less than about 100 mM, less than about 80 mM, less than about 60 mM, or less than about 50 mM. Compositions and systems may comprise an engineered effector protein and an assay excipient, which may stabilize a reagent or product, prevent aggregation or precipitation, or enhance or stabilize a detectable signal (e.g., a fluorescent signal). Examples of assay excipients include, but are not limited to, saccharides and saccharide derivatives (e.g., sodium carboxymethyl cellulose and cellulose acetate), detergents, glycols, polyols, esters, buffering agents, alginic acid, and organic solvents (e.g., DMSO).

In some instances, the naturally-occurring protein is a wildtype protein. An engineered protein may comprise a modified form of a naturally-occurring protein. The engineered protein may comprise one or more amino acid alterations (e.g., deletion, insertion, or substitution) relative to a naturally-occurring protein. In some instances, the engineered protein comprises or consists of an amino acid sequence that is at least 65%, at least 70%, at least 75%, at least 80%, at least 85%, at least 90%, at least 95%, at least 97%, at least 98%, or at least 99% identical to the amino acid sequence of a naturally-occurring protein. In some instances, the engineered protein comprises or consists of an amino acid sequence that is not greater than 90%, 91%, 92%, 93%, 94%, 95%, 96%, 97%, 98%, 99%, 99.5%, 99.9%, 99.95% or 99.99% identical to the amino acid sequence of a naturally-occurring protein.

In some instances, one or more amino acid alterations of the engineered protein relative to a naturally-occurring effector protein increase the nucleic acid-cleaving activity of the engineered protein relative to the naturally-occurring protein. The engineered protein may have at least about 10%, at least about 20%, at least about 30%, at least about 40%, at least about 50%, at least about 60%, at least about 70%, at least about 80%, at least about 90%, or at least about 100% greater nucleic acid-cleaving activity than the naturally-occurring protein. The engineered protein may have at least about 2 fold, at least about 3 fold, at least about 4 fold, at least about 5 fold, at least about 6 fold, at least about 7 fold, at least about 8 fold, at least about 9 fold, or at least about 10 fold of the nucleic acid-cleaving activity as compared to the naturally-occurring protein. The engineered protein may not have more than about 100-fold nucleic acid-cleaving activity as compared to the naturally-occurring protein.

In some instances, one or more amino acid alterations of the engineered protein relative to a naturally-occurring effector protein reduce the nucleic acid-cleaving activity of the engineered protein relative to the naturally-occurring protein. The engineered protein may have less than 90%, less than 80%, less than 70%, less than 60%, less than 50%, less than 40%, less than 30%, less than 20%, less than 10%, less than 5%, or less than 1% of the nucleic acid-cleaving activity as compared to the naturally-occurring protein. In some instances, engineered proteins have no substantial nucleic acid-cleaving activity. Engineered proteins may be enzymatically inactive or “dead,” that is it may bind to a nucleic acid but not cleave it. An enzymatically inactive protein may comprise an enzymatically inactive domain (e.g. inactive nuclease domain). An enzymatically inactive protein may associate with a guide nucleic acid to activate or repress transcription of a target nucleic acid sequence. In some instances, the enzymatically inactive protein is fused to a second protein.

### Fusion Proteins

In some instances, compositions of the instant disclosure comprise a fusion protein or uses thereof. In general, a fusion protein comprises an effector protein and a fusion partner protein that is heterologous to the effector protein. A fusion protein may also be referred to as a fusion effector protein or a fusion polypeptide. In general, the fusion partner protein is not an effector protein. In some embodiments, a fusion partner protein, also referred to simply as a fusion partner, comprises a polypeptide or peptide that is fused or linked to an effector protein. The fusion partner generally imparts some function to the fusion protein that is not provided by the effector protein. The fusion partner may provide a detectable signal. The fusion partner may modify a target nucleic acid. In some embodiments, the fusion partner makes a chemical modification to one or more nucleotides of a target nucleic acid. By way of non-limiting example, a fusion partner may modify a nucleobase of the target nucleic acid to an alternative nucleobase. The fusion partner may be capable of modulating expression of a target nucleic acid. The fusion partner may inhibit, reduce, activate or increase expression of a target nucleic acid. The fusion partner may interact with additional proteins to make modifications to a target nucleic acid.

In some instances, the fusion partner protein may be heterologous to the effector protein, and thus, referred to herein as a “heterologous protein.” A protein that is heterologous to the effector protein is a protein that is not covalently linked via an amide bond to the effector protein in nature. A heterologous protein may not be encoded by a species that encodes the effector protein. In some instances, the heterologous protein exhibits an activity (e.g., enzymatic activity) that it exhibits when it is fused to the effector protein. In some instances, the heterologous protein exhibits increased or reduced activity (e.g., enzymatic activity) when it is fused to the effector protein, relative to when it is not fused to the effector protein. In some instances, the heterologous protein exhibits an activity (e.g., enzymatic activity) that it does not exhibit when it is fused to the effector protein. In some instances, the effector protein and the fusion partner protein are linked through an amide bond. In some instances, the C-terminus of the effector protein is linked to the N terminus of the fusion partner protein. In some instances, the N-terminus of the effector protein is linked to the C-terminus of the fusion partner protein. A fusion partner protein is also simply referred to herein as a fusion partner. In some instances, the effector protein comprises an amino acid sequence selected from SEQ ID NOS: 1-1614 and 3195-3302. In some instances, the effector protein comprises an amino acid sequence that is at least 50%, at least 55%, at least 60%, at least 65%, at least 70%, at least 75%, at least 80%, at least 85%, at least 90%, at least 95%, at least 97%, at least 98%, at least 99%, or 100% identical to a sequence selected from SEQ ID NOS: 1-1614 and 3195-3302. In some instances, the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99%, or 100% identical to a sequence selected from SEQ ID NOS: 1-1614 and 3195-3302.

In some instances, a fusion partner provides enzymatic activity that modifies a protein (e.g., a histone) associated with a target nucleic acid. Such enzymatic activities include, but are not limited to, methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, de-ribosylation activity, myristoylation activity, and demyristoylation activity.

In some instances, fusion partners provide enzymatic activity that modifies a target nucleic acid. Such enzymatic activities include, but are not limited to, nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, and glycosylase activity. The target nucleic acid may comprise or consist of a ssRNA, dsRNA, ssDNA, or a dsDNA. In some instances, the fusion partner is a reverse transcriptase. In some instances, the fusion partner is a base editor. In general, a base editor comprises a deaminase that when fused with an effector protein changes a nucleobase to a different nucleobase, e.g., cytosine to thymine or guanine to adenine. In some instances, the base editor comprises a deaminase.

In some instances, the fusion partner modulates transcription (e.g., inhibits transcription, increases transcription) of a target nucleic acid. In some instances, the fusion partner is a protein (or a domain from a protein) that increases transcription, also referred to as a transcription activator. Transcriptional activators may promote transcription via recruitment of transcription activator proteins, modification of target DNA such as demethylation, recruitment of a DNA modifier, modulation of histones associated with target DNA, recruitment of a histone modifier such as those that modify acetylation and/or methylation of histones, or a combination thereof. Non-limiting examples of fusion partners that promote or increase transcription include, but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof.

In some instances, the fusion partner is a protein (or a domain from a protein) that inhibits transcription, also referred to as a transcriptional repressor. Transcriptional repressors may inhibit transcription via recruitment of transcription inhibitor proteins, modification of target DNA such as methylation, recruitment of a DNA modifier, modulation of histones associated with target DNA, recruitment of a histone modifier such as those that modify acetylation and/or methylation of histones, or a combination thereof. Non-limiting examples of fusion partners that decrease or inhibit transcription include, but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof.

In some instances, fusion proteins are targeted by a guide nucleic acid to a specific location in the target nucleic acid and exert locus-specific regulation such as blocking RNA polymerase binding to a promoter (which selectively inhibits transcription activator function), and/or modifying the local chromatin status (e.g., when a fusion sequence is used that modifies the target nucleic acid or modifies a protein associated with the target nucleic acid). In some instances, the modifications are transient (e.g., transcription repression or activation). In some instances, the modifications are inheritable. For instance, epigenetic modifications made to a target nucleic acid, or to proteins associated with the target nucleic acid, e.g., nucleosomal histones, in a cell, are observed in cells produced by proliferation of the cell.

In some instances, fusion proteins bind and/or cleave ssRNA. Non-limiting examples of fusion partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins. It is understood that a fusion protein may include the entire protein or in some instances may include a fragment of the protein (e.g., a functional domain). In some instances, the functional domain interacts with or binds ssRNA, including intramolecular and/or intermolecular secondary structures thereof, e.g., hairpins, stem-loops, etc.). The functional domain may interact transiently or irreversibly, directly or indirectly. Fusion proteins may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for repressing translation (e.g., Ago2 and Ago4); proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); proteins and protein domains responsible for RNA localization (e.g., from IMP1, ZBP1, She2p, She3p, and Bicaudal-D); proteins and protein domains responsible for nuclear retention of RNA (e.g., Rrp6); proteins and protein domains responsible for nuclear export of RNA (e.g., TAP, NXF1, THO, TREX, REF, and Aly); proteins and protein domains responsible for repression of RNA splicing (e.g., PTB, Sam68, and hnRNP A1); proteins and protein domains responsible for stimulation of RNA splicing (e.g., Serine/Arginine-rich (SR) domains); proteins and protein domains responsible for reducing the efficiency of transcription (e.g., FUS (TLS)); and proteins and protein domains responsible for stimulating transcription (e.g., CDK7 and HIV Tat). Alternatively, the effector domain may be a domain of a protein selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable fusion partner is a PUF RNA-binding domain, which is described in more detail in WO2012068627, which is hereby incorporated by reference in its entirety.

In some instances, the fusion partner comprises an RNA splicing factor. The RNA splicing factor may be used (in whole or as fragments thereof) for modular organization, with separate sequence-specific RNA binding modules and splicing effector domains. Non-limiting examples of RNA splicing factors include members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal Glycine-rich domain. Some splicing factors may regulate alternative use of splice site (ss) by binding to regulatory sequences between the two alternative sites. For example, ASF/SF2 may recognize ESEs and promote the use of intron proximal sites, whereas hnRNP A1 may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c\acute{ω}-elements that are located in either the core exon region or the exon extension region (i.e., between the two alternative 5′ splice sites). For more examples, see WO2010075303, which is hereby incorporated by reference in its entirety.

In some embodiments, an effector protein is a fusion protein, wherein the fusion protein comprises an effector protein (e.g., a Cas) and a fusion partner protein. In some embodiments, the effector protein comprises an amino acid sequence that is at least 70%, at least 75%, at least 80%, at least 85%, at least 90%, at least 95%, or 100% identical to any one of the sequences selected from SEQ ID NOS: 1-1614 and 3195-3302. In some embodiments, the amino acid of the effector protein is at least 70%, at least 75%, at least 80%, at least 85%, at least 90%, at least 95%, or 100% identical to any one of the sequences selected from SEQ ID NOS: 1-1614 and 3195-3302. Unless otherwise indicated, reference to effector proteins throughout the present disclosure include fusion proteins thereof.

In some embodiments, the fusion partner inhibits the formation of a multimeric complex of the effector protein. In some embodiments, the fusion partner promotes the formation of a multimeric complex of the effector protein. By way of non-limiting example, the fusion protein may comprise a Cas, and a fusion partner comprising a Calcineurin A tag, wherein the fusion protein dimerizes in the presence of Tacrolimus (FK506). Also by way of non-limiting example, the fusion protein may comprise a Cas and a SpyTag configured to dimerize or associate with another effector protein in a multimeric complex.

In some embodiments, fusion proteins and/or fusion partners comprise a prime editing enzyme. Such a fusion may be referred to as a prime editor. In some embodiments, a prime editor is capable of catalyzing the modification (insertion, deletion, or base-to-base conversion) of a target nucleic acid. A prime editing enzyme capable of catalyzing such a reaction is a reverse transcriptase. A non-limiting example of a reverse transcriptase is an M-MLV RT enzyme and variants thereof having polymerase activity. In some embodiments, the M-MLV RT enzyme comprises at least one mutation selected from D200N, L603W, T330P, T306K, and W313F relative to wildtype M-MLV RT enzyme. In general, a prime editing enzyme requires a prime editing guide RNA (pegRNA) to catalyze the modification. Such a pegRNA can be capable of identifying the nucleotide or nucleotide sequence in the target nucleic acid to be edited and encoding the new genetic information that replaces the targeted nucleotide or nucleotide sequence in the nucleic acid. In some embodiments, the target nucleic acid is a dsDNA molecule. In some embodiments, the pegRNA comprises a guide RNA comprising a first region that is bound by the effector protein, and a second region comprising a spacer sequence that is complementary to a target sequence of the target dsDNA molecule; a template RNA comprising a primer binding sequence that hybridizes to a primer sequence of the target dsDNA molecule that is formed when target nucleic acid is cleaved, and a template sequence that is complementary to at least a portion of the target sequence of the target dsDNA molecule with the exception of at least one nucleotide. In some embodiments, the spacer sequence is complementary to the target sequence on the target strand of the dsDNA molecule. In some embodiments, the spacer sequence is complementary to the target sequence on the non-target strand of the dsDNA molecule. In some instances, the primer binding sequence hybridizes to a primer sequence on the non-target strand of the target dsDNA molecule. In some instances, the primer binding sequence hybridizes to a primer sequence on the target strand of the target dsDNA molecule. In some instances, the target strand is cleaved. In some instances, the non-target strand is cleaved.

### Modifying Target Nucleic Acids

Disclosed herein are compositions and methods for modifying a target nucleic acid. The target nucleic acid may be a gene or a portion thereof. Methods and compositions may modify a coding portion of a gene, a non-coding portion of a gene, or a combination thereof. Modifying at least one gene using the compositions and methods described herein may reduce or increase expression of one or more genes. In some embodiments, compositions and methods reduce expression of one or more genes by at least 10%, at least 20%, at least 30%, at least 40%, at least 50%, at least 60%, at least 70%, at least 80%, at least 90%, or at least 95%. In some embodiments, compositions and methods remove all expression of a gene, also referred to as genetic knock out. In some embodiments, compositions and methods increase expression of one or more genes by at least 10%, at least 20%, at least 30%, at least 40%, at least 50%, at least 60%, at least 70%, at least 80%, at least 90%, or at least 100%.

### Base Editors.

In some embodiments, fusion partners modify a nucleobase of a target nucleic acid. Fusion proteins comprising such fusion partners and an effector protein may be referred to as base editors. When a base editor is described herein, it can refer to a fusion protein comprising a base editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

In some embodiments, base editors modify a sequence of a target nucleic acid. In some embodiments, base editors provide a nucleobase change in a DNA molecule. In some embodiments, the nucleobase change in the DNA molecule is selected from: an adenine (A) to guanine (G); cytosine (C) to thymine (T); and cytosine (C) to guanine (G). In some embodiments, base editors provide a nucleobase change in an RNA molecule. In some embodiments, the nucleobase change in the RNA molecule is selected from: adenine (A) to guanine (G); uracil (U) to cytosine (C); cytosine (C) to guanine (G); and guanine (G) to adenine (A). In some embodiments, the fusion partner is a deaminase, e.g., ADAR1/2.

Some base editors modify a nucleobase of on a single strand of DNA. In some embodiments, base editors modify a nucleobase on both strands of dsDNA. In some embodiments, upon binding to its target locus in DNA, base pairing between the guide nucleic acid and target DNA strand leads to displacement of a small segment of single-stranded DNA in an “R-loop”. In some embodiments, DNA bases within the R-loop are modified by the deaminase enzyme. In some embodiments, DNA base editors for improved efficiency in eukaryotic cells comprise a catalytically inactive effector protein that may generate a nick in the non-edited DNA strand, inducing repair of the non-edited strand using the edited strand as a template.

In some embodiments, a catalytically inactive effector protein can comprise an effector protein that is modified relative to a naturally-occurring nuclease to have a reduced or eliminated catalytic activity relative to that of the naturally-occurring nuclease, but retains its ability to interact with a guide nucleic acid. The catalytic activity that is reduced or eliminated is often a nuclease activity. The naturally-occurring nuclease may be a wildtype protein. In some embodiments, the catalytically inactive effector protein is referred to as a catalytically inactive variant of a nuclease, e.g., a Cas nuclease.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions comprise a base editor and a guide nucleic acid, wherein the guide nucleic acid directs the base editor to a sequence in a target gene. The target gene may be associated with a disease. In some embodiments, the guide nucleic acid directs that base editor to or near a mutation in the sequence of a target gene. The mutation may be the deletion of one more nucleotides. The mutation may be the addition of one or more nucleotides. The mutation may be the substitution of one or more nucleotides. The mutation may be the insertion, deletion or substitution of a single nucleotide, also referred to as a point mutation. The point mutation may be a SNP. The mutation may be associated with a disease. In some embodiments, the guide nucleic acid directs the base editor to bind a target sequence within the target nucleic acid that is within 0, 1, 2, 3, 4, 5, 6, 7, 8, 9, 10, 11, 12, 13, 14, 15, 16, 17, 18, 19, or 20 nucleotides of the mutation. In some embodiments, the guide nucleic acid comprises a sequence that is identical, complementary or reverse complementary to a target sequence of a target nucleic acid that comprises the mutation. In some embodiments, the guide nucleic acid comprises a sequence that is identical, complementary or reverse complementary to a target sequence of a target nucleic acid that is within 1, 2, 3, 4, 5, 6, 7, 8, 9, 10, 11, 12, 13, 14, 15, 16, 17, 18, 19, or 20 nucleotides of the mutation.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions comprise a base editor and a guide nucleic acid, wherein the guide nucleic acid directs the base editor to a sequence in a target gene.

In some embodiments, fusion partners comprise a base editing enzyme. When a base editing enzyme is described herein, it can refer to a protein, polypeptide, or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

In some embodiments, the base editing enzyme modifies the nucleobase of a deoxyribonucleotide. In some embodiments, the base editing enzyme modifies the nucleobase of a ribonucleotide. A base editing enzyme that converts a cytosine to a guanine or thymine may be referred to as a cytosine base editing enzyme. A base editing enzyme that converts an adenine to a to a guanine may be referred to as an adenine base editing enzyme. In some embodiments, the base editing enzyme comprises a deaminase enzyme. In some embodiments, the deaminase functions as a monomer. In some embodiments, the deaminase functions as heterodimer with an additional protein. In some embodiments, base editors comprise a DNA glycosylase inhibitor. In some embodiments, base editors comprise a uracil glycosylase inhibitor (UGI) or uracil N-glycosylase (UNG). In some embodiments, base editors do not comprise a UGI. In some embodiments, base editors do not comprise a UNG. In some embodiments, base editors do not comprise a functional fragment of a UGI. A functional fragment of a UGI is a fragment of a UGI that is capable of excising a uracil residue from DNA by cleaving an N-glycosydic bond. In some embodiments, a functional fragment comprises a fragment of a protein that retains some function relative to the entire protein. Non-limiting examples of functions are nucleic acid binding, protein binding, nuclease activity, nickase activity, deaminase activity, demethylase activity, or acetylation activity.

In some embodiments, a base editing enzyme can comprise a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase. In some embodiments, a base editor can be a fusion protein comprising a base editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

In some embodiments, the base editor is a cytidine deaminase base editor generated by ancestral sequence reconstruction as described in WO2019226953, which is hereby incorporated by reference in its entirety.

Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3:eaao4774 (2017), and Rees et al., Nat Rev Genet. 2018 December; 19(12):770-788. doi: 10.1038/s41576-018-0059-1, which are hereby incorporated by reference in their entirety.

In some embodiments, the base editor is a cytosine base editor (CBE). In general, a CBE comprises a cytosine base editing enzyme and a catalytically inactive effector protein. In some embodiments, the catalytically inactive effector protein is a catalytically inactive variant of an effector protein described herein. The CBE may convert a cytosine to a thymine. In some embodiments, the base editor is an adenine base editor (ABE). In general, an ABE comprises an adenine base editing enzyme and a catalytically inactive effector protein. In some embodiments, the catalytically inactive effector protein is a catalytically inactive variant of an effector protein described herein. The ABE generally converts an adenine to a guanine. In some embodiments, the base editor is a cytosine to guanine base editor (CGBE). In general, a CGBE converts a cytosine to a guanine.

In some embodiments, the base editor is a CBE. In some embodiments, the cytosine base editing enzyme is a cytidine deaminase. In some embodiments, the cytosine deaminase is an APOBEC1 cytosine deaminase, which accept ssDNA as a substrate but is incapable of cleaving dsDNA, fused to a catalytically inactive effector protein. In some embodiments, when bound to its cognate DNA, the catalytically inactive effector protein performs local denaturation of the DNA duplex to generate an R-loop in which the DNA strand not paired with the guide nucleic acid exists as a disordered single-stranded bubble. In some embodiments, the catalytically inactive effector protein generated ssDNA R-loop enables the CBE to perform efficient and localized cytosine deamination in vitro. In some examples, deamination activity is exhibited in a window of about 4 to about 10 base pairs. In some embodiments, fusion to the catalytically inactive effector protein presents the target site to APOBEC1 in high effective molarity, enabling the CBE to deaminate cytosines located in a variety of different sequence motifs, with differing efficacies. In some embodiments, the CBE is capable of mediating RNA-programmed deamination of target cytosines in vitro. In some embodiments, the CBE is capable of mediating RNA-programmed deamination of target cytosines in vivo. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C⋅G-to-G⋅C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise an uracil glycosylase inhibitor (UGI) or uracil N-glycosylase (UNG). In some embodiments, base excision repair (BER) of U⋅G in DNA is initiated by a UNG, which recognizes the U⋅G mismatch and cleaves the glyosidic bond between uracil and the deoxyribose backbone of DNA. In some embodiments, BER results in the reversion of the U⋅G intermediate created by the first CBE back to a C⋅G base pair. In some embodiments, UNG may be inhibited by fusion of uracil DNA glycosylase inhibitor (UGI), in some embodiments, a small protein from bacteriophage PBS, to the C-terminus of the CBE. In some embodiments, UGI is a DNA mimic that potently inhibits both human and bacterial UNG. In some embodiments, a UGI inhibitor is any protein or polypeptide that inhibits UNG. In some embodiments, the CBE mediates efficient base editing in bacterial cells and moderately efficient editing in mammalian cells, enabling conversion of a C⋅G base pair to a T⋅A base pair through a U⋅G intermediate. In some embodiments, the CBE is modified to increase base editing efficiency while editing more than one strand of DNA.

In some embodiments, the CBE nicks the non-edited DNA strand. In some embodiments, the non-edited DNA strand nicked by the CBE biases cellular repair of the U⋅G mismatch to favor a U⋅A outcome, elevating base editing efficiency. In some embodiments, the APOBEC1-nickase-UGI fusion efficiently edits in mammalian cells, while minimizing frequency of non-target indels.

In some embodiments, the cytidine deaminase is selected from APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, or saBE4-Gam as described in WO2021163587, WO2021087246, WO2021062227, and WO2020123887, which are incorporated herein by reference in their entirety.

In some embodiments, the fusion protein further comprises a non-protein uracil-DNA glcosylase inhibitor (npUGI). In some embodiments, the npUGI is selected from a group of small molecule inhibitors of uracil-DNA glycosylase (UDG), or a nucleic acid inhibitor of UDG. In some embodiments, the non-protein uracil-DNA glcosylase inhibitor (npUGI) is a small molecule derived from uracil. Examples of small molecule non-protein uracil-DNA glcosylase inhibitors, fusion proteins, and Cas-CRISPR systems comprising base editing activity are described in WO2021087246, which is incorporated by reference in its entirety,

In some embodiments, the fusion partner is a deaminase, e.g., ADAR1/2, ADAR-2, or AID. In some embodiments, the base editor is an ABE. In some embodiments, the adenine base editing enzyme of the ABE is an adenosine deaminase. In some embodiments, the adenine base editing enzyme is selected from ABE8e, ABE8.20m, APOBEC3A, Anc APOBEC (a.k.a. AncBE4Max), and BtAPOBEC2. In some embodiments, the ABE base editor is an ABE7 base editor. In some embodiments, the deaminase or enzyme with deaminase activity is selected from ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, or ABE8.24d. In some embodiments, the adenine base editing enzyme is ABE8.1d. In some embodiments, the adenosine base editor is ABE9. Exemplary deaminases are described in US20210198330, WO2021041945, WO2021050571A1, and WO2020123887, all of which are incorporated herein by reference in their entirety. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871. Additional examples of deaminase domains are also described in WO2018027078 and WO2017070632, which are hereby incorporated by reference in their entirety.

In some embodiments, an ABE converts an A⋅T base pair to a G⋅C base pair. In some embodiments, the ABE converts a target A⋅T base pair to G⋅C in vivo. In some embodiments, the ABE converts a target A⋅T base pair to G⋅C in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. In some embodiments, an ABE comprises an engineered adenosine deaminase enzyme capable of acting on ssDNA.

In some embodiments, a base editor comprises an adenosine deaminase variant that differs from a naturally occurring deaminase. Relative to the naturally occurring deaminase, the adenosine deaminase variant may comprise a V82S alteration, a T166R alteration, or a combination thereof. In some embodiments, the adenosine deaminase variant comprises at least one of the following alterations relative to a naturally occurring adenosine deaminase: Y147T, Y147R, Q154S, Y123H, and Q154R., which are incorporated herein by reference in their entirety.

In some embodiments, a base editor comprises a deaminase dimer. In some embodiments, a base editor is a deaminase dimer further comprising a base editing enzyme and an adenine deaminase (e.g., TadA).

In some embodiments, the adenosine deaminase is a TadA monomer (e.g., Tad*7.10, TadA*8 or TadA*9). In some embodiments, the adenosine deaminase is a TadA*8 variant. Such a TadA*8 variant includes TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety. In some embodiments, a base editor is a deaminase dimer comprising a base editing enzyme fused to TadA via a linker.

In some embodiments, the amino terminus of the fusion partner protein is linked to the carboxy terminus of the effector protein via the linker. In some embodiments, the carboxy terminus of the fusion partner protein is linked to the amino terminus of the effector protein via the linker.

In some embodiments, the base editing enzyme is fused to TadA at the N-terminus. In some embodiments, the base editing enzyme is fused to TadA at the C-terminus. In some embodiments, the base editing enzyme is a deaminase dimer comprising an ABE. In some embodiments, the deaminase dimer comprises an adenosine deaminase. In some embodiments, the deaminase dimer comprises TadA fused to an adenine base editing enzyme selected from ABE8e, ABE8.20m, APOBEC3A, Anc APOBEC (a.k.a. AncBE4Max), and BtAPOBEC2. In some embodiments TadA is fused to ABE8e or a variant thereof. In some embodiments TadA is fused to ABE8e or a variant thereof at the amino-terminus (ABE8e-TadA). In some embodiments, TadA is fused to ABE8e or a variant thereof at the carboxy terminus (ABE8e-TadA).

### Recombinases

In some embodiments, the fusion partners comprise a recombinase domain. In some embodiments, the enzymatically inactive protein is fused with a recombinase. In some embodiments, the recombinase is a site-specific recombinase. In some embodiments, the fusion partners comprise a recombinase domain wherein the recombinase is a site-specific recombinase. In some embodiments, described herein is a programmed nuclease comprising reduced nuclease activity or no nuclease activity and fused with a recombinase, wherein the recombinase can be a site-specific recombinase. Such polypeptides can be used for site-directed transgene insertion. Examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include, but are not limited to, gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include, but are not limited to: Bxb1, wBeta, BL3, phiR4, A118, TG1, MR11, phi370, SPBc, TP901-1, phiRV, FC1, K38, phiBT1, and phiC31. Further discussion and examples of suitable recombinase fusion partners are described in U.S. Pat. No. 10,975,392, which is incorporated herein by reference in its entirety.

In some embodiments, the fusion protein comprises a linker that links the recombinase domain to the Cas-CRISPR domain of the effector protein. In some embodiments, the linker is The-Ser.

### Modifying Proteins

In some embodiments, a fusion partner provides enzymatic activity that modifies a protein (e.g., a histone) associated with a target nucleic acid. Such enzymatic activities include, but are not limited to, methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, de-ribosylation activity, myristoylation activity, and demyristoylation activity.

In some embodiments, the fusion partner has enzymatic activity that modifies a protein associated with a target nucleic acid. The protein may be a histone, an RNA binding protein, or a DNA binding protein. Examples of such protein modification activities include methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

### CRISPRa Fusions and CRISPRi Fusions

In some embodiments, fusion partners activate or increase expression of a target nucleic acid. Fusion proteins comprising such fusion partners and an effector protein may be referred to as CRISPRa fusions. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator. In some embodiments, a transcriptional activator can describe a polypeptide or a fragment thereof that can activate or increase transcription of a target nucleic acid molecule. Transcriptional activators may promote transcription via: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof.

In some embodiments, fusions partners inhibit or reduce expression of a target nucleic acid. Fusion proteins comprising such fusion partners and an effector protein may be referred to as CRISPRi fusions. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. In some embodiments, a transcriptional repressor can describe a polypeptide or a fragment thereof that is capable of arresting, preventing, or reducing transcription of a target nucleic acid. Transcriptional repressors may inhibit transcription via: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

### Additional Fusion Partners

In some embodiments, the fusion partner is a chloroplast transit peptide (CTP), also referred to as a plastid transit peptide. In some embodiments, this targets the fusion protein to a chloroplast. Chromosomal transgenes from bacterial sources must have a sequence encoding a CTP sequence fused to a sequence encoding an expressed protein if the expressed protein is to be compartmentalized in the plant plastid (e.g. chloroplast). The CTP is removed in a processing step during translocation into the plastid. Accordingly, localization of an exogenous protein to a chloroplast is often accomplished by means of operably linking a polynucleotide sequence encoding a CTP sequence to the 5′ region of a polynucleotide encoding the exogenous protein. In some embodiments, the CTP is located at the N-terminus of the fusion protein. Processing efficiency may, however, be affected by the amino acid sequence of the CTP and nearby sequences at the amino terminus (NH2 terminus) of the peptide.

In some embodiments, the fusion partner is an endosomal escape peptide. In some embodiments, an endosomal escape protein comprises the amino acid sequence GLFXALLXLLXSLWXLLLXA, wherein each X is independently selected from lysine, histidine, and arginine. In some embodiments, an endosomal escape protein comprises the amino acid sequence GLFHALLHLLHSLWHLLLHA. In some embodiments, the amino acid sequence of the endosomal escape protein is GLFXALLXLLXSLWXLLLXA or GLFHALLHLLHSLWHLLLHA.

Further suitable fusion partners include, but are not limited to, proteins (or fragments/domains thereof) that are boundary elements (e.g., CTCF), proteins and fragments thereof that provide periphery recruitment (e.g., Lamin A, Lamin B, etc.), protein docking elements (e.g., FKBP/FRB, Pil1/Aby1, etc.).

### Linkers for Fusion Partners

In some embodiments, fusion proteins comprise an effector protein and a fusion partner protein. The effector protein may be fused or linked to the fusion partner protein. The terms “fused” and “linked” may be used interchangeably. In some instances, the effector protein and the fusion partner are directly linked via a covalent bond. In some instances, effector proteins and fusion partners of a fusion effector protein are connected via a linker. The linker may comprise or consist of a covalent bond. The linker may comprise or consist of a chemical group. In some embodiments, the linker comprises an amino acid. In some embodiments, a linker comprises a bond or molecule that links a first polypeptide to a second polypeptide. In some embodiments, a peptide linker comprises at least two amino acids linked by an amide bond. In general, the linker connects a terminus of the effector protein to a terminus of the fusion partner. In some embodiments, the carboxy terminus of the effector protein is linked to the amino terminus of the fusion partner. In some embodiments, the carboxy terminus of the fusion partner is linked to the amino terminus of the effector protein.

In some embodiments, a terminus of the effector protein is linked to a terminus of the fusion partner through an amide bond. In some embodiments, a terminus of the effector protein is linked to a terminus of the fusion partner through a peptide bond. In some instances, linkers comprise an amino acid. In some embodiments, linkers comprise a peptide. In some embodiments, an effector protein is coupled to a fusion partner via a linker protein. The linker protein may have any of a variety of amino acid sequences. A linker protein may comprise a region of rigidity (e.g., beta sheet, alpha helix), a region of flexibility, or any combination thereof. In some embodiments, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. In some embodiments, when a linked amino acids is described herein, it can refer to at least two amino acids linked by an amide bond.

These linkers may be produced by using synthetic, linker-encoding oligonucleotides to couple the proteins, or may be encoded by a nucleic acid sequence encoding a fusion protein (e.g., an effector protein coupled to a fusion partner). Examples of linker proteins include glycine polymers (G)n, glycine-serine polymers (including, for example, (GS)n, GSGGSn, GGSGGSn, and GGGSn, where n is an integer of at least one), glycine-alanine polymers, and alanine-serine polymers. Exemplary linkers may comprise amino acid sequences including, but not limited to, GGSG, GGSGG, GSGSG, GSGGG, GGGSG, and GSSSG.

In some instances, linkers do not comprise an amino acid. In some instances, linkers do not comprise a peptide. In some instances, linkers comprise a nucleotide, a polynucleotide, a polymer, or a lipid.

### Nuclease-Dead Effector Proteins

In some embodiments, the effector protein can comprise an enzymatically inactive and/or “dead” (abbreviated by “d”) effector protein in combination (e.g., fusion) with a polypeptide comprising recombinase activity. Although an effector protein normally has nuclease activity, in some embodiments, an effector protein does not have nuclease activity. In some embodiments, an effector protein comprising at least 65%, at least 70%, at least 75%, at least 80%, at least 85%, at least 90%, at least 92%, at least 95%, at least 97%, at least 98%, at least 99%, or 100% sequence identity with any one of the sequences selected from SEQ ID NOS: 1-1614 and 3195-3302 is a nuclease-dead effector protein. In some embodiments, the effector protein comprising at least 65%, at least 70%, at least 75%, at least 80%, at least 85%, at least 90%, at least 92%, at least 95%, at least 97%, at least 98%, at least 99%, or 100% sequence identity with any one of the sequences selected from SEQ ID NOS: 1-1614 and 3195-3302 is modified or engineered to be a nuclease-dead effector protein.

The effector protein can comprise a modified form of a wild type counterpart. The modified form of the wild type counterpart can comprise an amino acid change (e.g., deletion, insertion, or substitution) that reduces the nucleic acid-cleaving activity of the effector protein. For example, a nuclease domain (e.g., HEPN domain) of an effector polypeptide can be deleted or mutated so that it is no longer functional or comprises reduced nuclease activity. The modified form of the effector protein can have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. The modified form of an effector protein can have no substantial nucleic acid-cleaving activity. When an effector protein is a modified form that has no substantial nucleic acid-cleaving activity, it can be referred to as enzymatically inactive and/or dead. A dead effector polypeptide can bind to a target sequence but may not cleave the target nucleic acid. A dead effector polypeptide can associate with a guide nucleic acid to activate or repress transcription of a target nucleic acid.

In some embodiments, the effector proteins disclosed herein (e.g. an effector protein of a CRISPR-Cas system, or a Cas protein) are modified relative to a naturally-occurring effector protein to have reduced or eliminated catalytic activity relative to that of the naturally-occurring effector protein, but retain their ability to interact with a guide nucleic acid. For example, a “dCas” protein refers to any one of the Cas proteins disclosed herein that is modified relative to a naturally-occurring Cas protein to have a reduced or eliminated catalytic activity relative to that of the naturally-occurring effector protein, but retains its ability to interact with a guide nucleic acid.

### Effector Protein Activity

The effector proteins of the present disclosure may show an enhanced activity (e.g., nucleic acid binding activity, nuclease activity), when measured in a cleavage assay or a reporter assay, under certain conditions relative to a control condition. For example, the effector proteins of the present disclosure may have variable levels of activity based on a buffer formulation, a pH level, temperature, or salt. Buffers consistent with the present disclosure include phosphate buffers, Tris buffers, and HEPES buffers. Effector proteins of the present disclosure can show optimal activity in phosphate buffers, Tris buffers, and HEPES buffers.

Effector proteins can also exhibit varying levels of activity at different pH levels. For example, enhanced nuclease activity can be observed between pH 7 and pH 9. In some embodiments, effector proteins of the present disclosure exhibit enhanced cleavage at about pH 7, about pH 7.1, about pH 7.2, about pH 7.3, about pH 7.4, about pH 7.5, about pH 7.6, about pH 7.7, about pH 7.8, about pH 7.9, about pH 8, about pH 8.1, about pH 8.2, about pH 8.3, about pH 8.4, about pH 8.5, about pH 8.6, about pH 8.7, about pH 8.8, about pH 8.9, about pH 9, from pH 7 to 7.5, from pH 7.5 to 8, from pH 8 to 8.5, from pH 8.5 to 9, or from pH 7 to 8.5.

In some embodiments, the effector proteins of the present disclosure exhibit activity or enhanced activity at a temperature of 25° C. to 50° C. in the presence of target nucleic acid. For example, the effector proteins of the present disclosure can exhibit enhanced cleavage of an ssDNA-FQ reporter at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., from 30° C. to 40° C., from 35° C. to 45° C., or from 35° C. to 40° C.

In some embodiments, the effector proteins of the present disclosure exhibit activity or enhanced activity under a salt concentration from 25 nM salt to 200 mM salt. Non-limiting examples of such salts are NaCl and KCl. The effector proteins of the present disclosure can be active at salt concentrations of from 25 nM to 500 nM salt, from 500 nM to 1000 nM salt, from 1000 nM to 2000 nM salt, from 2000 nM to 3000 nM salt, from 3000 nM to 4000 nM salt, from 4000 nM to 5000 nM salt, from 5000 nM to 6000 nM salt, from 6000 nM to 7000 nM salt, from 7000 nM to 8000 nM salt, from 8000 nM to 9000 nM salt, from 9000 nM to 0.01 mM salt, from 0.01 mM to 0.05 mM salt, from 0.05 mM to 0.1 mM salt, from 0.1 mM to 10 mM salt, from 10 mM to 100 mM salt, or from 100 mM to 500 mM salt. The effector proteins of the present disclosure can exhibit cleavage activity independent of the salt concentration in a sample.

In some embodiments, the effector proteins of the present disclosure exhibit activity or enhanced activity in the presence of a co-factor. In certain embodiments, the co-factor allows the effector proteins to perform a function. In some embodiments, the function is pre-crRNA processing and/or target nucleic acid cleavage. As discussed in Jiang F. and Doudna J. A. (Annu. Rev. Biophys. 2017. 46:505-29), Cas9 uses divalent metal ions as co-factors. The suitability of a divalent metal ion as a cofactor can easily be assessed, such as by methods based on those described by Sundaresan et al. (Cell Rep. 2017 Dec. 26; 21(13): 3728-3739). In some embodiments, the co-factor is a divalent metal ion. Exemplary divalent metal ions include: Mg²⁺, Mn²⁺, Zn²⁺, Ca²⁺, and Cu²⁺. In some embodiments, an effector protein forms a complex with a divalent metal ion. In preferred embodiments, an effector protein forms a complex with Mg²⁺, Mn²⁺, Zn²⁺, Ca²⁺, or Cu².

### Synthesis, Isolation and Assaying

Effector proteins of the present disclosure may be synthesized, using any suitable method. Effector proteins of the present disclosure may be produced in vitro or by eukaryotic cells or by prokaryotic cells. Effector proteins can be further processed by unfolding, e.g. heat denaturation, dithiothreitol reduction, etc. and may be further refolded, using any suitable method.

Methods of generating and assaying the effector proteins described herein are well known to one of skill in the art. Examples of such methods are described in the Examples provided herein. Any of a variety of methods can be used to generate an effector protein disclosed herein. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, M D (1999); Gillman et al., Directed Evolution Library Creation: Methods and Protocols (Methods in Molecular Biology) Springer, 2nd ed (2014)). One non-limiting example of a method for preparing an effector protein is to express recombinant nucleic acids encoding the effector protein in a suitable microbial organism, such as a bacterial cell, a yeast cell, or other suitable cell, using methods well known in the art.

In some embodiments, an effector protein provided herein is an isolated effector protein. In some embodiments, effector proteins described herein can be isolated and purified for use in compositions, systems, and/or methods described herein. Methods described here can include the step of isolating effector proteins described herein. An isolated effector protein provided herein can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

For example, compositions and/or systems described herein can further comprise a purification tag that can be attached to an effector protein, or a nucleic acid encoding for a purification tag that can be attached to a nucleic acid encoding for an effector protein as described herein. A purification tag, as used herein, can be an amino acid sequence which can attach or bind with high affinity to a separation substrate and assist in isolating the protein of interest from its environment, which can be its biological source, such as a cell lysate. Attachment of the purification tag can be at the N or C terminus of the effector protein. Furthermore, an amino acid sequence recognized by a protease or a nucleic acid encoding for an amino acid sequence recognized by a protease, such as TEV protease or the HRV3C protease can be inserted between the purification tag and the effector protein, such that biochemical cleavage of the sequence with the protease after initial purification liberates the purification tag. Purification and/or isolation can be through high performance liquid chromatography (HPLC), exclusion chromatography, gel electrophoresis, affinity chromatography, or other purification technique. Examples of purification tags are as described herein.

In some embodiments, effector proteins described herein are isolated from cell lysate. In some embodiments, the compositions described herein can comprise 20% or more by weight, 75% or more by weight, 95% or more by weight, or 99.5% or more by weight of an effector protein, related to the method of preparation of compositions described herein and its purification thereof, wherein percentages can be upon total protein content in relation to contaminants. Thus, in some embodiments, an effector protein described herein is at least 80% pure, at least 85% pure, at least 90% pure, at least 95% pure, at least 98% pure, or at least 99% pure (e.g., free of contaminants, non-engineered polypeptide proteins or other macromolecules, etc.).

### V. Multimeric Complexes

Compositions, systems, and methods of the present disclosure may comprise a multimeric complex or uses thereof, wherein the multimeric complex comprises multiple effector proteins that non-covalently interact with one another. A multimeric complex may comprise enhanced activity relative to the activity of any one of its effector proteins alone. For example, a multimeric complex comprising two effector proteins may comprise greater nucleic acid binding affinity and/or nuclease activity than that of either of the effector proteins provided in monomeric form under the same conditions that the activity with the multimeric complex was observed. A multimeric complex may have an affinity for a target sequence of a target nucleic acid and is capable of catalytic activity (e.g., cleaving, nicking or modifying the nucleic acid) at or near the target sequence. Multimeric complexes may be activated when complexed with a guide nucleic acid. Multimeric complexes may be activated when complexed with a guide nucleic acid and a target nucleic acid. In some instances, the multimeric complex cleaves the target nucleic acid. In some instances, the multimeric complex nicks the target nucleic acid. Effector proteins of a multimeric complex may target different nucleic acid sequences. Effector proteins of a multimeric complex may target different types of nucleic acids. By way of non-limiting example, a first effector protein may target double- and single-stranded nucleic acids, and a second effector protein may only target single-stranded nucleic acids).

In some instances, the multimeric complex is a dimer comprising two effector proteins of identical amino acid sequences. In some instances, the multimeric complex comprises a first effector protein and a second effector protein, wherein the amino acid sequence of the first effector protein is at least 90%, at least 92%, at least 94%, at least 96%, at least 98% identical, or at least 99% identical to the amino acid sequence of the second effector protein.

In some instances, the multimeric complex is a heterodimeric complex comprising at least two effector proteins of different amino acid sequences. In some instances, the multimeric complex is a heterodimeric complex comprising a first effector protein and a second effector protein, wherein the amino acid sequence of the first effector protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second effector protein.

Various aspects of the present disclosure include compositions and methods comprising multiple effector proteins, and uses thereof, respectively. An effector protein comprising at least 65% sequence identity to any one of the sequences selected from SEQ ID NOS: 1-1614 and 3195-3302 may be provided with a second effector protein. Two effector proteins may target different nucleic acid sequences. Two effector proteins may target different types of nucleic acids (e.g., a first effector protein may target double- and single-stranded nucleic acids, and a second effector protein may only target single-stranded nucleic acids).

In some embodiments, multimeric complexes comprise at least one effector protein, or a fusion protein thereof, comprising an amino acid sequence with at least 65%, at least 70%, at least 75%, at least 80%, at least 85%, at least 90%, at least 95% or 100% identity to any one of the sequences selected from SEQ ID NOS: 1-1614 and 3195-3302. In some embodiments, multimeric complexes comprise at least one effector protein or a fusion protein thereof, wherein the amino acid sequence of the effector protein is at least 65%, at least 70%, at least 75%, at least 80%, at least 85%, at least 90%, at least 95% or 100% identical to any one of the sequences selected from SEQ ID NOS: 1-1614 and 3195-3302.

### VI. Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. In general, a guide nucleic acid is a nucleic acid molecule, at least a portion of which may be bound by an effector protein, thereby forming a ribonucleoprotein complex (RNP). In some embodiments, the guide nucleic acid imparts activity or sequence selectivity to the effector protein. When complexed with an effector protein, guide nucleic acids can bring the effector protein into proximity of a target nucleic acid. The guide nucleic acid may hybridize to a target nucleic acid or a portion thereof. In some embodiments, when a guide nucleic acid and an effector protein form an RNP, at least a portion of the RNP binds, recognizes, and/or hybridizes to a target nucleic acid. Those skilled in the art in reading the below specific examples of guide nucleic acids as used in RNPs described herein, will understand that in some embodiments, a RNP can hybridize to one or more target sequences in a target nucleic acid, thereby allowing the RNP to modify and/or recognize a target nucleic acid or sequence contained therein or to modify and/or recognize non-target sequences depending on the guide nucleic acid, and in some embodiments, the effector protein, used.

Guide nucleic acids may also be referred to herein as a “guide RNA.” However, a guide nucleic acid, including guide RNAs described herein, may comprise various modified nucleotides and deoxyribonucleotides. The term “guide RNA,” as well as crRNA and tracrRNA, includes nucleic acids comprising ribonucleosides, deoxyribonucleosides and biochemically or chemically modified nucleotides (e.g., one or more sequence modifications as described herein), and any combinations thereof. Modified nucleosides may comprise modified sugar moieties (e.g., 2′-OMe, 2′-fluoro, and constrained ethyl), modified nucleobases or modified backbone linkages (phosphorothioate linkage).

The guide nucleic acid may comprise a first region complementary to a target nucleic acid (FR1) and a second region that is not complementary to the target nucleic acid (FR2). In some cases, FR1 is located 5′ to FR2 (FR1-FR2). In some cases, FR2 is located 5′ to FR1 (FR2-FR1). In some embodiments, the first region comprises a spacer region, wherein the spacer region can interact in a sequence-specific manner with (e.g., has complementarity with, or can hybridize to) a target nucleic acid. In certain embodiments, the second region comprises a repeat region that interacts with the effector protein.

A guide nucleic acid may comprise a naturally occurring sequence. A guide nucleic acid may comprise a non-naturally occurring sequence, wherein the sequence of the guide nucleic acid, or any portion thereof, may be different from the sequence of a naturally occurring nucleic acid. The guide nucleic acid may be chemically synthesized or recombinantly produced. Guide nucleic acids and portions thereof may be found in or identified from a CRISPR array present in the genome of a host organism or cell. In some embodiments, an effector protein or a multimeric complex thereof cleaves a precursor RNA (“pre-crRNA”) to produce a guide RNA, also referred to as a “mature guide RNA.” An effector protein that cleaves pre-crRNA to produce a mature guide RNA is said to have pre-crRNA processing activity. In some embodiments, a repeat region of a guide RNA comprises mutations or truncations relative to respective regions in a corresponding pre-crRNA.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. Multiple guide nucleic acids may target an effector protein to different locations in the target nucleic acid by binding to different portions of the target nucleic acid. A first guide nucleic acid may bind or cleave a first portion of a target nucleic acid and a second guide nucleic acid may bind or cleave a second portion of the target nucleic acid. The first portion and the second portion of the target nucleic acid may be located at least 10, at least 20, at least 30, at least 40, at least 50, at least 60, at least 70, at least 80, at least 90 or at least 100 nucleotides apart. The first portion and the second portion of the target nucleic acid may be located between 100 and 200, 200 and 300, 300 and 400, 400 and 500, 500 and 600, 600 and 700, 700 and 800, 800 and 900 or 900 and 1000 nucleotides apart. In some instances, the first portion and/or the second portion of the target nucleic acid are located in an intron of a gene. In some instances, the first portion and/or the second portion of the target nucleic acid are located in an exon of a gene. In some instances, the first portion and/or the second portion of the target nucleic acid span and exon-intron junction of a gene. In some instances, the first portion and/or the second portion of the target nucleic acid are located on either side of an exon and cutting at both sites results in deletion of the exon. In some embodiments, composition, systems and methods comprise a donor nucleic acid that may be inserted in replacement of a deleted or cleaved sequence of the target nucleic acid. In some embodiments, compositions, systems and methods comprising multiple guide nucleic acids or uses thereof comprise multiple different effector proteins.

In some embodiments, a guide nucleic acid comprises 10, 11, 12, 13, 14, 15, 16, 17, 18, 19, 20, 21, 22, 23, 24, 25, 26, 27, 28, 29, or 30 linked nucleotides. In general, a guide nucleic acid comprises at least 10 linked nucleotides. In some embodiments, a guide nucleic acid comprises at least 25 linked nucleotides. A guide nucleic acid may comprise 10 to 50 linked nucleotides. In some embodiments, the guide nucleic acid comprises or consists essentially of about 12 to about 80 linked nucleotides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleotides. In some embodiments, the guide nucleic acid has about 10 to about 60, about 20 to about 50, or about 30 to about 40 linked nucleotides.

In some embodiments, a guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a sequence of nucleotides that is present in a host eukaryotic cell. Such a sequence of nucleotides is distinguished from nucleotide sequences present in other host cells, such as prokaryotic cells, or viruses. Said sequences present in a eukaryotic cell can be located a gene, an exon, an intron, a non-coding (e.g., promoter or enhancer) region, a selectable marker, tag, signal, and the like.

In some embodiments, the guide nucleic acid can bind to a target sequence, wherein the target sequence is eukaryotic. The guide nucleic acid may bind to a target nucleic acid, such as DNA or RNA, from a cancer gene or gene associated with a genetic disorder, or an amplicon thereof, as described herein. The guide nucleic acid may bind to a target nucleic acid such as a nucleic acid from a bacterium, a virus, a parasite, a protozoon, a fungus or other agents responsible for a disease, or an amplicon thereof. The target nucleic acid may comprise a mutation, such as a single nucleotide polymorphism (SNP). A mutation may confer for example, resistance to a treatment, such as antibiotic treatment.

In general, the guide nucleic acid comprises a CRISPR RNA (crRNA) that is at least partially complementary to a target nucleic acid. In some instances, the guide nucleic acid comprises a trans-activating crRNA (tracrRNA), at least a portion of which interacts with the effector protein. In general, tracrRNA comprises a first sequence that is capable of being non-covalently bound by an effector protein. TracrRNAs may comprise a second sequence that hybridizes to a portion of a crRNA, which may be referred to as a repeat hybridization sequence. The tracrRNA may hybridize to a portion of the guide nucleic acid that does not hybridize to the target nucleic acid. In some instances, the crRNA and tracrRNA are provided as a single guide nucleic acid, also referred to as a single guide RNA (sgRNA). In some instances, a crRNA and tracrRNA function as two separate, unlinked molecules.

In some instances, guide nucleic acids do not comprise a tracrRNA. In some cases, effector proteins do not require a tracrRNA to locate and/or cleave a target nucleic acid. In some instances, the crRNA of the guide nucleic acid comprises a repeat region and a spacer region, wherein the repeat region binds to the effector protein and the spacer region hybridizes to a target sequence of the target nucleic acid. The repeat sequence of the crRNA may interact with the effector protein, allowing for the guide nucleic acid and the effector protein to form an RNP complex.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

### Spacer Region

In general, guide nucleic acids comprise a spacer sequence within the spacer region that hybridizes with a target sequence of a target nucleic acid. The spacer sequence may comprise complementarity with a target sequence of a target nucleic acid. The spacer sequence can function to direct the guide nucleic acid to the target nucleic acid for detection and/or modification. The spacer sequence may be complementary to a target sequence that is adjacent to a PAM that is recognizable by an effector protein of interest.

In some embodiments, the spacer region is 15-28 linked nucleotides in length. In some embodiments, the spacer region is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides in length. In some embodiments, the spacer region is 18-24 linked nucleotides in length. In some embodiments, the spacer region is at least 15 linked nucleotides in length. In some embodiments, the spacer region is at least 16, 18, 20, or 22 linked nucleotides in length. In some embodiments, the spacer region comprises at least 10, 11, 12, 13, 14, 15, 16, 17, 18, 19, 20, 21, 22, 23, 24, 25, 26, 27, 28, 29, or 30 nucleotides. In some embodiments, the spacer region is at least 17 linked nucleotides in length. In some embodiments, the spacer region is at least 18 linked nucleotides in length. In some embodiments, the spacer region is at least 20 linked nucleotides in length. In some embodiments, the spacer region is at least 80%, at least 85%, at least 90%, at least 95% or 100% complementary to a target sequence of the target nucleic acid. In some embodiments, the spacer region is 100% complementary to the target sequence of the target nucleic acid. In some embodiments, the spacer region comprises at least 15 contiguous nucleotides that are complementary to the target nucleic acid. It is understood that the sequence of a spacer region need not be 100% complementary to that of a target sequence of a target nucleic acid to hybridize or hybridize specifically to the target sequence. The spacer sequence may comprise at least one nucleotide that is not complementary to the corresponding nucleotide of the target sequence.

### Repeat Region

Guide nucleic acids can also comprise a repeat region that interacts with the effector protein. The repeat region may also be referred to as a “protein-binding segment.” Typically, the repeat region is adjacent to the spacer region. For example, a guide nucleic acid that interacts with the effector protein may comprise a repeat region that is 5′ of the spacer region. In certain embodiments, the repeat region is followed by the spacer region in the 5′ to 3′ direction. In some embodiments, the repeat region is between 10 and 50, 12 and 48, 14 and 46, 16 and 44, and 18 and 42 nucleotides in length. In certain embodiments, the repeat region is between 19 and 37 nucleotides in length.

In some embodiments, the spacer sequence and the direct repeat sequence(s) of the guide nucleic acid are present within the same molecule. In some embodiments, the spacer and repeat sequences are linked directly to one another. In some embodiments, a linker is present between the spacer and repeat sequences. The linker may comprise 1, 2, 3, 4, 5, 6, 7, 8, 9 or 10 nucleotides. The linker may comprise more than 10 nucleotides. In some instances, the linker comprises the sequence 5′-GAAA-3′. In some embodiments, the spacer sequence and the repeat sequence(s) of the guide nucleic acid are present in separate molecules, which are joined to one another by base pairing interactions.

In some embodiments, the protein binding segment includes two sequences that are complementary to each other and hybridize to form a double stranded RNA duplex (dsRNA duplex). In some instances, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming region can include a bulge. In certain embodiments, the repeat region comprises a hairpin or stem-loop structure, optionally at the 5′ portion of the repeat region. In some embodiments, a strand of the stem portion comprises a sequence and the other strand of the stem portion comprises a sequence that is, at least partially, complementary. In certain embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

In some embodiments, guide nucleic acids comprise a nucleotide sequence as described herein (e.g., Columns B1, B2, and B3 of TABLE 1 and Columns D1 and D2 of TABLE 2). Such nucleotide sequences described herein may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein (e.g., Columns B1, B2, and B3 of TABLE 1 and Columns D1 and D2 of TABLE 2) also discloses the complementary nucleotide sequence, the reverse nucleotide sequence, and the reverse complement nucleotide sequence, any one of which can be a nucleotide sequence for use in a guide nucleic acid as described herein.

In some embodiments, the guide nucleic acid comprises a sequence that is at least 70%, at least 80%, at least 85%, at least 90%, at least 95%, or 100% identical to any one of the sequences recited in Columns B1, B2, and B3 of TABLE 1 and Columns D1 and D2 of TABLE 2 or any combination thereof.

In some embodiments, the guide nucleic acid comprises a sequence that is at least 70%, at least 75%, at least 80%, at least 85%, at least 90%, at least 95%, or 100% identical to any one of the sequences of Columns B1, B2, and B3 of TABLE 1. In some embodiments, the guide nucleic acid comprises a sequence that is at least 70%, at least 75%, at least 80%, at least 85%, at least 90%, at least 95%, or 100% identical to any one of the sequences of Columns D1 and D2 of TABLE 2. In some embodiments, the guide nucleic acid comprises a sequence that is at least 70%, at least 75%, at least 80%, at least 85%, at least 90%, at least 95%, or 100% identical to any one of the sequences of Columns B1, B2, and B3 of TABLE 1 and a sequence that is at least 70%, at least 75%, at least 80%, at least 85%, at least 90%, at least 95%, or 100% identical to any one of the sequences of Columns D1 and D2 of TABLE 2.

The guide nucleic acid may bind to a target nucleic acid (e.g., a single strand of a target nucleic acid) or a portion thereof. The guide nucleic acid may bind to a target nucleic acid such as a nucleic acid from a bacterium, a virus, a parasite, a protozoa, a fungus or other agents responsible for a disease, or an amplicon thereof. The target nucleic acid may comprise a mutation, such as a single nucleotide polymorphism (SNP). A mutation may confer for example, resistance to a treatment, such as antibiotic treatment. The guide nucleic acid may bind to a target nucleic acid, such as DNA or RNA, from a cancer gene or gene associated with a genetic disorder, or an amplicon thereof, as described herein.

In some cases, the guide comprises 10, 11, 12, 13, 14, 15, 16, 17, 18, 19, 20, 21, 22, 23, 24, 25, 26, 27, 28, 29, or 30 linked nucleosides. In general, a guide nucleic acid comprises at least linked nucleosides. In some instances, a guide nucleic acid comprises at least 25 linked nucleosides. A guide nucleic acid may comprise 10 to 50 linked nucleosides. In some cases, the guide nucleic acid comprises or consists essentially of about 12 to about 80 linked nucleosides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleosides. In some cases, the guide nucleic acid has about 10 to about 60, about 20 to about 50, or about 30 to about 40 linked nucleosides.

A guide nucleic acid can generally comprise a crRNA, at least a portion of which is complementary to a target sequence of a target nucleic acid. In some embodiments, the composition comprising an effector protein and a guide nucleic acid further comprises a tracrRNA that interacts with the effector protein. In some embodiments, the crRNA and the tracrRNA are covalently linked. In some embodiments, the crRNA and tracrRNA are linked by a phosphodiester bond. In some embodiments, the crRNA and tracrRNA are linked by one or more linked nucleotides. In some embodiments, a crRNA and tracrRNA function as two separate, unlinked molecules. In some embodiments, compositions, systems and methods does not comprise or require a tracrRNA. In some embodiments, an effector protein does not require a tracrRNA to locate and/or cleave a target nucleic acid. In some embodiments, the crRNA of the guide nucleic acid comprises a repeat region and a spacer region, wherein the repeat region binds to the effector protein and the spacer region hybridizes to a target sequence of the target nucleic acid. The repeat sequence of the crRNA may interact with an effector protein, allowing for the guide nucleic acid and the effector protein to form an RNP complex. In some embodiments, the guide nucleic acid is a sgRNA.

In some instances, compositions and systems provided herein comprise a nucleic acid expression vector encoding an effector protein. In some instances, compositions and systems comprise multiple nucleic acid expression vectors. In some instances, at least one of the multiple nucleic acid expression vectors encodes a guide nucleic acid described herein. In some instances, the guide nucleic acid and the effector protein are encoded by the same nucleic acid expression vector. In some instances, the engineered guide nucleic acid and the effector protein are encoded by different nucleic acid expression vectors. In some instances, a nucleic acid expression vector may encode one or more guide nucleic acids.

crRNA

In general, a crRNA comprises a spacer region that hybridizes to a target sequence of a target nucleic acid. In some embodiments, the crRNA of the guide nucleic acid comprises a repeat region and a spacer region, wherein the repeat region binds to the effector protein and the spacer region hybridizes to a target sequence of the target nucleic acid. The repeat sequence of the crRNA may interact with an effector protein, allowing for the guide nucleic acid and the effector protein to form an RNP complex. Accordingly in some embodiments, the guide nucleic acid is a crRNA.

A crRNA may be the product of processing of a longer precursor CRISPR RNA (pre-crRNA) transcribed from the CRISPR array by cleavage of the pre-crRNA within each direct repeat sequence to afford shorter, mature crRNAs. A crRNA may be generated by a variety of mechanisms, including the use of dedicated endonucleases (e.g., Cas6 or Cas5d in Type I and III systems), coupling of a host endonuclease (e.g., RNase III) with tracrRNA (Type II systems), or a ribonuclease activity endogenous to the effector protein itself (e.g., Cpfl, from Type V systems). A crRNA may also be specifically generated outside of processing of a pre-crRNA and individually contacted to an effector protein in vivo or in vitro.

In some embodiments, the length of the crRNA is not greater than about 40, about 45, about 50, about 55, about 60, about 65, about 70 or about 75 linked nucleosides. In some embodiments, the length of the crRNA is about 30 to about 120 linked nucleosides. In some embodiments, the length of a crRNA is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleosides. In some embodiments, the length of a crRNA is about 40, about 45, about 50, about 55, about 60, about 65, about 70 or about 75 linked nucleosides.

tracrRNA

In some embodiments, the compositions comprising a guide nucleic acid and an effector protein (e.g., in a dual nucleic acid system) comprises a tracrRNA. A tracrRNA may include deoxyribonucleosides, ribonucleosides, chemically modified nucleosides, or any combination thereof. A tracrRNA may be separate from, but form a complex with, a guide nucleic acid and an effector protein. A tracrRNA may include a nucleotide sequence that hybridizes with a portion of a guide nucleic acid (e.g., a repeat hybridization region). A tracrRNA may also form a secondary structure (e.g., one or more hairpin loops) that facilitates the binding of an effector protein to a guide nucleic acid and/or modification activity of an effector protein on a target nucleic acid (e.g., a hairpin region). A tracrRNA may include a repeat hybridization region and a hairpin region. The repeat hybridization region may hybridize to all or part of the repeat sequence of a guide nucleic acid. The repeat hybridization region may be positioned 3′ of the hairpin region. The hairpin region may include a first sequence, a second sequence that is reverse complementary to the first sequence, and a stem-loop linking the first sequence and the second sequence.

In some embodiments, tracrRNAs comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the tracrRNA comprises a pseudoknot (e.g., a secondary structure comprising a stem at least partially hybridized to a second stem or half-stem secondary structure). An effector protein may recognize a tracrRNA comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the tracrRNA comprises at least 2, at least 3, at least 4, or at least 5 stem regions.

In some embodiments, the length of a tracrRNA is not greater than 50, 56, 68, 71, 73, 95, or 105 linked nucleotides. In some embodiments, the length of a tracrRNA is about 30 to about 120 linked nucleotides. In some embodiments, the length of a tracrRNA is about 50 to about 105, about 50 to about 95, about 50 to about 73, about 50 to about 71, about 50 to about 68, or about 50 to about 56 linked nucleotides. In some embodiments, the length of a tracrRNA is 56 to 105 linked nucleotides, from 56 to 105 linked nucleotides, 68 to 105 linked nucleotides, 71 to 105 linked nucleotides, 73 to 105 linked nucleotides, or 95 to 105 linked nucleotides. In some embodiments, the length of a tracrRNA is 40 to 60 nucleotides. In some embodiments, the length of a tracrRNA is 50, 56, 68, 71, 73, 95, or 105 linked nucleotides. In some embodiments, the length of a tracrRNA is 50 nucleotides.

An exemplary tracrRNA may comprise, from 5′ to 3′, a 5′ region, a hairpin region, a repeat hybridization region, and a 3′ region. In some embodiments, the 5′ region may hybridize to the 3′ region. In some embodiments, the 5′ region does not hybridize to the 3′ region. In some embodiments, the 3′ region is covalently linked to the crRNA (e.g., through a phosphodiester bond). In some embodiments, a tracrRNA may comprise an un-hybridized region at the 3′ end of the tracrRNA. The un-hybridized region may have a length of about 1, about 2, about 3, about 4, about 5, about 6, about 7, about 8, about 9, about 10, about 12, about 14, about 16, about 18, or about 20 linked nucleotides. In some embodiments, the length of the un-hybridized region is 0 to 20 linked nucleotides.

sgRNA

In some embodiments, compositions comprise a guide nucleic acid and an effector protein without a tracrRNA (e.g., a single guide nucleic acid system), wherein the guide nucleic acid is referred to herein as a sgRNA. A sgRNA may include deoxyribonucleosides, ribonucleosides, chemically modified nucleosides, or any combination thereof. A sgRNA may also include a nucleotide sequence that forms a secondary structure (e.g., one or more hairpin loops) that facilitates the binding of an effector protein to the sgRNA and/or modification activity of an effector protein on a target nucleic acid (e.g., a hairpin region). Such a sequence can be contained within a handle sequence as described herein. A sgRNA may include a handle sequence having a hairpin region, as well as a linker and a repeat sequence. The sgRNA having a handle sequence can have a hairpin region positioned 3′ of the linker and/or repeat sequence. The sgRNA having a handle sequence can have a hairpin region positioned 5′ of the linker and/or repeat sequence. The hairpin region may include a first sequence, a second sequence that is reverse complementary to the first sequence, and a stem-loop linking the first sequence and the second sequence.

In some embodiments, the handle sequence of a sgRNA comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the sgRNA comprises a pseudoknot (e.g., a secondary structure comprising a stem at least partially hybridized to a second stem or half-stem secondary structure). An effector protein may recognize a sgRNA comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the sgRNA comprises at least 2, at least 3, at least 4, or at least 5 stem regions.

In some embodiments, the length of a handle sequence in a sgRNA is not greater than 50, 56, 66, 67, 68, 69, 70, 71, 72, 73, 95, or 105 linked nucleotides. In some embodiments, the length of a handle sequence in a sgRNA is about 30 to about 120 linked nucleotides. In some embodiments, the length of a handle sequence in a sgRNA is about 50 to about 105, about 50 to about 95, about 50 to about 73, about 50 to about 71, about 50 to about 70, or about 50 to about 69 linked nucleotides. In some embodiments, the length of a handle sequence in a sgRNA is 56 to 105 linked nucleotides, from 56 to 105 linked nucleotides, 66 to 105 linked nucleotides, 67 to 105 linked nucleotides, 68 to 105 linked nucleotides, 69 to 105 linked nucleotides, 70 to 105 linked nucleotides, 71 to 105 linked nucleotides, 72 to 105 linked nucleotides, 73 to 105 linked nucleotides, or 95 to 105 linked nucleotides. In some embodiments, the length of a handle sequence in a sgRNA is 40 to 70 nucleotides. In some embodiments, the length of a handle sequence in a sgRNA is 50, 56, 66, 67, 68, 69, 70, 71, 72, 73, 95, or 105 linked nucleotides. In some embodiments, the length of a handle sequence in a sgRNA is 69 nucleotides.

An exemplary handle sequence in a sgRNA may comprise, from 5′ to 3′, a 5′ region, a hairpin region, and a 3′ region. In some embodiments, the 5′ region may hybridize to the 3′ region. In some embodiments, the 5′ region does not hybridize to the 3′ region. In some embodiments, the 3′ region is covalently linked to a spacer sequence (e.g., through a phosphodiester bond). In some embodiments, the 5′ region is covalently linked to a spacer sequence (e.g., through a phosphodiester bond).

### Pooling Guide Nucleic Acids

In some embodiments, compositions, systems or methods provided herein comprise a pool of guide nucleic acids. In some embodiments, the pool of guide nucleic acids were tiled against a target nucleic acid, e.g., the genomic locus of interest or uses thereof. In some embodiments, a guide nucleic acid is selected from a group of guide nucleic acids that have been tiled against a nucleic acid sequence of a genomic locus of interest. The genomic locus of interest may belong to a viral genome, a bacterial genome, or a mammalian genome. Non-limiting examples of viral genomes are an HPV genome, an HIV genome, an influenza genome, or a coronavirus genome. Often, these guide nucleic acids are pooled for detecting a target nucleic acid in a single assay. Pooling of guide nucleic acids may ensure broad spectrum identification, or broad coverage, of a target species within a single reaction. This may be particularly helpful in diseases or indications, like sepsis, that may be caused by multiple organisms. The pool of guide nucleic acids may enhance the detection of a target nucleic using systems of methods described herein relative to detection with a single guide nucleic acid. The pool of guide nucleic acids may ensure broad coverage of the target nucleic acid within a single reaction using the methods described herein. In some embodiments, the pool of guide nucleic acids are collectively complementary to at least 50%, at least 55%, at least 60%, at least 65%, at least 70%, at least 75%, at least 80%, at least 85%, at least 90%, at least 95% or 100% of the target nucleic acid. In some embodiments, at least a portion of the guide nucleic acids of the pool overlap in sequence. In some embodiments, at least a portion of the guide nucleic acids of the pool do not overlap in sequence. In some embodiments, the pool of guide nucleic acids comprises at least 2, at least 3, at least 4, at least 5, or at least 6 guide nucleic acids targeting different sequences of a target nucleic acid.

### VII. Sequence Modifications

Polypeptides (e.g., effector proteins) and nucleic acids (e.g., engineered guide nucleic acids) described herein can be further modified as described throughout and as further described herein. Examples are modifications of interest that do not alter primary sequence, including chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Modifications disclosed herein can also include modification of described polypeptides and/or engineered guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g. D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or engineered guide nucleic acids described herein. For example, groups can be introduced during synthesis or during expression of a polypeptide (e.g., an effector protein), which allow for linking to other molecules or to a surface. Thus, e.g., cysteines can be used to make thioethers, histidines for linking to a metal ion complex, carboxyl groups for forming amides or esters, amino groups for forming amides, and the like.

Modifications can further include modification of nucleic acids described herein (e.g., engineered guide nucleic acids) to provide the nucleic acid with a new or enhanced feature, such as improved stability. Such modifications of a nucleic acid include a base modification, a backbone modification, a sugar modification, or combinations thereof, of one or more nucleotides, nucleosides, or nucleobases in a nucleic acid.

In some embodiments, nucleic acids (e.g., engineered guide nucleic acids) described herein comprise one or more modifications comprising: 2′O-methyl modified nucleotides, 2′ Fluoro modified nucleotides; locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH2-NH—O—CH2-, —CH2-N(CH3)-O—CH2- (known as a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O—CH2-); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH2 component parts; and combinations thereof.

### VIII. Vectors and Multiplexed Expression Vectors

In some embodiments, compositions and systems provided herein comprise a vector system encoding a polypeptide (e.g., an effector protein) described herein. In some embodiments, compositions and systems provided herein comprise a vector system encoding a guide nucleic acid (e.g., crRNA) described herein. In some embodiments, compositions and systems provided herein comprise a multi-vector system encoding an effector protein and a guide nucleic acid described herein, wherein the guide nucleic acid and the effector protein are encoded by the same or different vectors. In some embodiments, the engineered guide and the engineered effector protein are encoded by different vectors of the system. In some embodiments, a nucleic acid encoding a polypeptide (e.g., an effector protein) comprises an expression vector. In some embodiments, a nucleic acid encoding a polypeptide is a messenger RNA. In some embodiments, an expression vector comprises or encodes an engineered guide nucleic acid. In some embodiments, the expression vector encodes the crRNA.

In some embodiments, a vector may encode one or more engineered effector proteins. In some embodiments, a vector may encode 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 engineered effector proteins. In some embodiments, a vector can encode one or more engineered effector proteins comprising an amino acid sequence of any one of the sequences selected from SEQ ID NOS: 1-1614 and 3195-3302.

In some embodiments, a vector may encode one or more guide nucleic acids. In some embodiments, a vector may encode 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 different guide nucleic acids.

In some embodiments, a vector can comprise or encode one or more regulatory elements. Regulatory elements can refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector can comprise or encode for one or more additional elements, such as, for example, replication origins, antibiotic resistance (or a nucleic acid encoding the same), a tag (or a nucleic acid encoding the same), selectable markers, and the like.

Vectors described herein can encode a promoter—a regulatory region on a nucleic acid, such as a DNA sequence, capable of initiating transcription of a downstream (3′ direction) coding or non-coding sequence. As used herein, a promoter can be bound at its 3′ terminus to a nucleic acid the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. A promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors can be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human H1 promoter (H1). By transcriptional activation, it is intended that transcription will be increased above basal levels in the target cell by 10 fold, by 100 fold, or by 1000 fold, or more. In addition, vectors used for providing a nucleic acid that, when transcribed, produces an engineered guide nucleic acid and/or a nucleic acid that encodes an effector protein to a cell may include nucleic acid sequences that encode for selectable markers in the target cells, so as to identify cells that have taken up the engineered guide nucleic acid and/or an effector protein.

In general, vectors provided herein comprise at least one promotor or a combination of promoters driving expression or transcription of one or more genome editing tools described herein. In some embodiments, the viral vector comprises a nucleotide sequence of a promoter. In some embodiments, the viral vector comprises two promoters. In some embodiments, the viral vector comprises three promoters. In some embodiments, the length of the promoter is less than about 500, less than about 400, or less than about 300 linked nucleotides. In some embodiments, the length of the promoter is at least 100 linked nucleotides. Non-limiting examples of promoters include CMV, 7SK, EF1a, RPBSA, hPGK, EFS, SV40, PGK1, Ubc, human beta actin promoter, CAG, TRE, UAS, Ac5, Polyhedrin, CaMKIIa, GAL1, H1, TEF1, GDS, ADH1, CaMV35S, Ubi, U6, MNDU3, MSCV, Ck8e, SPC5-12, Desmin, MND and CAG.

In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, an effector protein (or a nucleic acid encoding same) and/or an engineered guide nucleic acid (or a nucleic acid that, when transcribed, produces same) are coadministered with a donor nucleic acid. Coadministration can be contact with a target nucleic acid, administered to a cell, such as a host cell, or administered as method of nucleic acid detection, editing, and/or treatment as described herein, in a single vehicle, such as a single expression vector. In certain embodiments, an effector protein (or a nucleic acid encoding same) and/or an engineered guide nucleic acid (or a nucleic acid that, when transcribed, produces same) are not co-administered with donor nucleic acid in a single vehicle. In certain embodiments, an effector protein (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

### Lipid Particles

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector. In some embodiments, a lipid or a lipid nanoparticle can encapsulate the effector protein, the sgRNA or crRNA, the nucleic acid encoding the effector protein and/or the DNA molecule encoding the sgRNA or crRNA. LNPs are effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3):146-157). In some embodiments, a method can comprise contacting a cell with an expression vector. In some embodiments, contacting can comprise electroporation, lipofection, or lipid nanoparticle (LNP) delivery of an expression vector.

### Viral Vectors

An expression vector can be a viral vector. In some embodiments, a viral vector comprises a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. In some embodiments, the expression vector is an adeno-associated viral vector. There are a variety of viral vectors that are associated with various types of viruses, including but not limited to retroviruses (e.g., lentiviruses and y-retroviruses), adenoviruses, arenaviruses, alphaviruses, adeno-associated viruses (AAVs), baculoviruses, vaccinia viruses, herpes simplex viruses and poxviruses. A viral vector provided herein can be derived from or based on any such virus. Often the viral vectors provided herein are an adeno-associated viral vector (AAV vector). Generally, an AAV vector has two inverted terminal repeats (ITRs). According, in some embodiments, the viral vector provided herein comprises two inverted terminal repeats of AAV. The DNA sequence in between the ITRs of an AAV vector provided herein may be referred to herein as the sequence encoding the genome editing tools. These genome editing tools can include, but are not limited to, an effector protein, effector protein modifications (e.g., nuclear localization signal (NLS), polyA tail), guide nucleic acid(s), respective promoter(s), and a donor nucleic acid, or combinations thereof. In some embodiments, a nuclear localization signal comprises an entity (e.g., peptide) that facilitates localization of a nucleic acid, protein, or small molecule to the nucleus, when present in a cell that contains a nuclear compartment.

In general, viral vectors provided herein comprise at least one promotor or a combination of promoters driving expression or transcription of one or more genome editing tools described herein. In some embodiments, the length of the promoter is less than about 500, less than about 400, or less than about 300 linked nucleotides. In some embodiments, the length of the promoter is at least 100 linked nucleotides. Non-limiting examples of promoters include CMV, EF1a, RPBSA, hPGK, EFS, SV40, PGK1, Ubc, human beta actin promoter, CAG, TRE, UAS, Ac5, Polyhedrin, CaMKIIa, GAL1, H1, TEF1, GDS, ADH1, CaMV35S, Ubi, U6, MNDU3, and MSCV. In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44.

In some embodiments, the coding region of the AAV vector forms an intramolecular double-stranded DNA template thereby generating an AAV vector that is a self-complementary AAV (scAAV) vector. In general, the sequence encoding the genome editing tools of an scAAV vector has a length of about 2 kb to about 3 kb. The scAAV vector can comprise nucleotide sequences encoding an effector protein, providing guide nucleic acids described herein, and a donor nucleic acid described herein. In some embodiments, the AAV vector provided herein is a self-inactivating AAV vector.

In some embodiments, an AAV vector provided herein comprises a modification, such as an insertion, deletion, chemical alteration, or synthetic modification, relative to a wild-type AAV vector.

In some embodiments, the viral particle that delivers the viral vector described herein is an AAV. AAVs are characterized by their serotype. Non-limiting examples of AAV serotypes are AAV1, AAV2, AAV3, AAV4, AAV5, AAV6, AAV7, AAV8, AAV9, AAV10, AAV11, AAV12, scAAV, AAV-rh10, chimeric or hybrid AAV, or any combination, derivative, or variant thereof.

### Producing AAV Particles

The AAV particles described herein can be referred to as recombinant AAV (rAAV). Often, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E40RF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles can be produced in the mammalian cell culture system by providing the rAAV plasmid to the mammalian cell. In some embodiments, producing rAAV virus particles in a mammalian cell can comprise transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, an insect cell for producing rAAV viral particles comprises a Sf9 cell. In some embodiments, production of rAAV virus particles in insect cells can comprise baculovirus. In some embodiments, production of rAAV virus particles in insect cells can comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety

### IX. Systems

Disclosed herein, in some aspects, are systems for modifying a nucleic acid, comprising any one of the effector proteins described herein, or a multimeric complex thereof. In some instances, systems comprise a guide nucleic acid described herein. Systems may be used to detect, modify, or edit a target nucleic acid.

In some embodiments, the effector proteins having reduced or eliminated catalytic activity relative to that of the naturally-occurring effector protein, but having the ability to interact with a guide nucleic acid disclosed herein (such as, a dCas protein) associates with, or forms a complex with an engineered transposon system to provide a CRISPR-associated transposase (CAST) system for improved RNA-guided DNA integration. The engineered transposon system may comprise one or more transposase proteins, including a functional TnsB protein, a functional TnsC protein and/or a functional TniQ protein. In some embodiments, the engineered transposon system comprises a functional TnsB protein, a functional TnsC protein and a functional TniQ protein. In some embodiments, the engineered transposon system lacks a functional TnsA protein. Further details on transposases that may be used in association with the effector proteins disclosed herein are provided in Peters J E, et al., Proc Natl Acad Sci USA. 2017 Aug. 29; 114(35):E7358-E7366; Strecker, J. et al. Science 365, 48-53 (2019); Saito M, et al., Cell. 2021 Apr. 29; 184(9):2441-2453.e18; Petassi M T, et al., Cell. 2020 Dec. 23; 183(7):1757-1771, and Klompe S E, et al., Nature. 2019 July; 571(7764):219-225, the contents of each of which are herein incorporated by reference in their entireties for all purposes. In some embodiments, the effector proteins disclosed herein are capable of directing the engineered transposon system to a target insertion site of the target nucleic acid.

In some embodiments, the disclosed system for RNA-guided DNA integration comprises the following elements: (a) a CRISPR-Cas system, comprising: (i) any one of the dCas proteins disclosed herein, and (ii) a guide RNA (gRNA); (b) an engineered transposon system, wherein the engineered transposon system comprises a functional TnsB protein, a functional TnsC protein and a functional TniQ protein, but lacks a functional TnsA protein; (c) at least one nicking effector protein (e.g. at least one effector protein having nickase activity disclosed herein, or the nickase domain therefrom, and/or one or more vectors encoding the effector protein having nickase activity); and (d) at least one donor nucleic acid to be integrated into a target nucleic acid.

In some embodiments, the effector protein with nickase activity disclosed herein is fused to the TnsB protein. In yet other embodiments, the CRISPR-Cas system comprises a effector protein having nickase activity disclosed herein.

In the RNA-guided DNA integration systems disclosed herein, the 3′ nicking activity of TnsB is combined with the catalytic activity (e.g. 5′ nicking activity) of an effector protein having nickase activity disclosed herein to catalyze the “cut and paste” insertion of the donor nucleic acid into the target site in a targeted, RNA-mediated manner through the function of any one of the dCas proteins disclosed herein. Thus, the RNA-guided DNA integration systems disclosed herein exhibit not only the simplicity and orientation predictability characteristic of the Type V-K CRISPR-associated transposase (CAST) systems, but also the on-target specificity and “cut-and-paste” capability of the Type I CAST systems.

Systems may be used to modify the activity or expression of a target nucleic acid. In some instances, systems comprise an effector protein described herein, a reagent, support medium, or a combination thereof. In some instances, systems comprise a fusion protein described herein. In some instances, effector proteins comprise an amino acid sequence that is at least 70%, at least 75%, at least 80%, at least 85%, at least 90%, at least 95%, or 100% identical to any one of the amino acid sequences selected from SEQ ID NOS: 1-1614 and 3195-3302. In some instances, the amino acid sequence of the effector protein is at least 70%, at least 75%, at least 80%, at least 85%, at least 90%, at least 95%, or 100% identical to any one of the amino acid sequences selected from SEQ ID NOS: 1-1614 and 3195-3302.

In some embodiments, systems comprise an effector protein described herein, a guide nucleic acid described herein, a reagent, support medium, or a combination thereof. In some embodiments, the effector protein comprises an effector protein, or a fusion protein thereof, described herein. In some embodiments, effector protein comprises an amino acid sequence that is at least 65%, at least 70%, at least 75%, at least 80%, at least 85%, at least 90%, at least 95%, or 100% identical to any one of the sequences selected from SEQ ID NOS: 1-1614 and 3195-3302. In some embodiments, the amino acid sequence of the effector protein is at least 65%, at least 70%, at least 75%, at least 80%, at least 85%, at least 90%, at least 95%, or 100% identical to any one of the sequences selected from SEQ ID NOS: 1-1614 and 3195-3302.

Systems may be used for detecting the presence of a target nucleic acid associated with or causative of a disease, such as cancer, a genetic disorder, or an infection. In some instances, systems are useful for phenotyping, genotyping, or determining ancestry. In some instances, systems are useful for modifying a target nucleic acid associated with or causative of a disease, such as cancer, a genetic disorder, or an infection. Unless specified otherwise, systems include kits and may be referred to as kits. Unless specified otherwise, systems include devices and may also be referred to as devices. Systems described herein may be provided in the form of a companion diagnostic assay or device, a point-of-care assay or device, or an over-the-counter diagnostic assay/device.

Reagents and effector proteins of various systems may be provided in a reagent chamber or on the support medium. Alternatively, the reagent and/or effector protein may be contacted with the reagent chamber or the support medium by the individual using the system. An exemplary reagent chamber is a test well or container. The opening of the reagent chamber may be large enough to accommodate the support medium. Optionally, the system comprises a buffer and a dropper. The buffer may be provided in a dropper bottle for ease of dispensing. The dropper may be disposable and transfer a fixed volume. The dropper may be used to place a sample into the reagent chamber or on the support medium.

### System Solutions

In general, systems comprise a solution in which the activity of an effector protein occurs. Often, the solution comprises or consists essentially of a buffer. The solution or buffer may comprise a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some instances, a buffer is required for cell lysis activity or viral lysis activity.

In some instances, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. In some instances, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, or 9.5 to 10.5.

In some instances, systems comprise a solution, wherein the solution comprises at least one salt. In some instances, the at least one salt is selected from potassium acetate, magnesium acetate, sodium chloride, potassium chloride, magnesium chloride, calcium chloride, and any combination thereof. In some instances, the concentration of the at least one salt in the solution is 5 mM to 100 mM, 5 mM to 10 mM, 1 mM to 60 mM, or 1 mM to 10 mM. In some instances, the concentration of the at least one salt is about 105 mM. In some instances, the concentration of the at least one salt is about 55 mM. In some instances, the concentration of the at least one salt is about 7 mM. In some instances, the solution comprises potassium acetate and magnesium acetate. In some instances, the solution comprises sodium chloride and magnesium chloride. In some instances, the solution comprises potassium chloride and magnesium chloride. In some instances, the salt is a magnesium salt and the concentration of magnesium in the solution is at least 5 mM, 7 mM, at least 9 mM, at least 11 mM, at least 13 mM, or at least 15 mM. In some instances, the concentration of magnesium is less than 20 mM, less than 18 mM or less than 16 mM.

In some instances, systems comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some instances, the crowding agent is glycerol. In some instances, the concentration of the crowding agent in the solution is 0.01% (v/v) to 10% (v/v). In some instances, the concentration of the crowding agent in the solution is 0.5% (v/v) to 10% (v/v).

In some instances, systems comprise a solution, wherein the solution comprises at least one detergent. Exemplary detergents include Tween, Triton-X, and IGEPAL. A solution may comprise Tween, Triton-X, or any combination thereof. A solution may comprise Triton-X. A solution may comprise IGEPAL CA-630. In some instances, the concentration of the detergent in the solution is 2% (v/v) or less. In some instances, the concentration of the detergent in the solution is 1% (v/v) or less. In some instances, the concentration of the detergent in the solution is 0.00001% (v/v) to 0.01% (v/v). In some instances, the concentration of the detergent in the solution is about 0.01% (v/v).

In some instances, systems comprise a solution, wherein the solution comprises at least one reducing agent. Exemplary reducing agents comprise dithiothreitol (DTT), B-mercaptoethanol (BME), or tris(2-carboxyethyl) phosphine (TCEP). In some instances, the reducing agent is DTT. In some instances, the concentration of the reducing agent in the solution is 0.01 mM to 100 mM. In some instances, the concentration of the reducing agent in the solution is 0.1 mM to 10 mM. In some instances, the concentration of the reducing agent in the solution is 0.5 mM to 2 mM. In some instances, the concentration of the reducing agent in the solution is 0.01 mM to 100 mM. In some instances, the concentration of the reducing agent in the solution is 0.1 mM to 10 mM. In some instances, the concentration of the reducing agent in the solution is about 1 mM.

In some instances, systems comprise a solution, wherein the solution comprises a competitor. In general, competitors compete with the target nucleic acid or the reporter nucleic acid for cleavage by the effector protein or a dimer thereof. Exemplary competitors include heparin, and imidazole, and salmon sperm DNA. In some instances, the concentration of the competitor in the solution is 1 μg/mL to 100 μg/mL. In some instances, the concentration of the competitor in the solution is 40 μg/mL to 60 μg/mL.

In some instances, systems comprise a solution, wherein the solution comprises a co-factor. In some instances, the co-factor allows an effector protein or a multimeric complex thereof to perform a function, including pre-crRNA processing and/or target nucleic acid cleavage. The suitability of a cofactor for an effector protein or a multimeric complex thereof may be assessed, such as by methods based on those described by Sundaresan et al. (Cell Rep. 2017 Dec. 26; 21(13): 3728-3739). In some instances, an effector or a multimeric complex thereof forms a complex with a co-factor. In some instances, the co-factor is a divalent metal ion. In some instances, the divalent metal ion is selected from Mg²⁺, Mn²⁺, Zn²⁺, Ca²⁺, Cu²⁺. In some instances, the divalent metal ion is Mg²⁺. In some instances, the co-factor is Mg²⁺.

### Reporters

In some instances, systems disclosed herein comprise a reporter. By way of non-limiting and illustrative example, a reporter may comprise a single stranded nucleic acid and a detection moiety (e.g., a labeled single stranded RNA reporter), wherein the nucleic acid is capable of being cleaved by an effector protein (e.g., a CRISPR/Cas protein as disclosed herein) or a multimeric complex thereof, releasing the detection moiety, and, generating a detectable signal. As used herein, “reporter” is used interchangeably with “reporter nucleic acid” or “reporter molecule.” The effector proteins disclosed herein, activated upon hybridization of a guide nucleic acid to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded.

In some instances, reporters comprise a protein capable of generating a signal. A signal may be a calorimetric, potentiometric, amperometric, optical (e.g., fluorescent, colorimetric, etc.), or piezo-electric signal. In some instances, the reporter comprises a detection moiety. Suitable detectable labels and/or moieties that may provide a signal include, but are not limited to, an enzyme, a radioisotope, a member of a specific binding pair; a fluorophore; a fluorescent protein; a quantum dot; and the like.

In some instances, the reporter comprises a detection moiety and a quenching moiety. In some instances, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some instances, the quenching moiety is 5′ to the cleavage site and the detection moiety is 3′ to the cleavage site. In some instances, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some instances, the detection moiety is at the 5′ terminus of the nucleic acid of a reporter. In some instances, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, (3-glucuronidase, invertase, Xanthine Oxidase, firefly luciferase, and glucose oxidase (GO).

In some instances, the detection moiety comprises an invertase. The substrate of the invertase may be sucrose. A DNS reagent may be included in the system to produce a colorimetric change when the invertase converts sucrose to glucose. In some instances, the reporter nucleic acid and invertase are conjugated using a heterobifunctional linker via sulfo-SMCC chemistry.

Suitable fluorophores may provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Non-limiting examples of fluorophores are fluorescein amidite, 6-Fluorescein, IRDye 700, TYE 665, Alex Fluor 594, or ATTO TM 633 (NHS Ester). The fluorophore may be an infrared fluorophore. The fluorophore may emit fluorescence in the range of 500 nm and 720 nm. In some instances, the fluorophore emits fluorescence at a wavelength of 700 nm or higher. In other cases, the fluorophore emits fluorescence at about 665 nm. In some instances, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some instances, the fluorophore emits fluorescence in the range 450 nm to 750 nm, 500 nm to 650 nm, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some instances, the quenching moiety quenches a detection moiety that emits fluorescence at a wavelength of 700 nm or higher. In other cases, the quenching moiety quenches a detection moiety that emits fluorescence at about 660 nm or about 670 nm. In some instances, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some instances, the quenching moiety quenches a detection moiety that emits fluorescence in the range 450 nm to 750 nm, 500 nm to 650 nm, or 550 to 650 nm. A quenching moiety may quench fluorescein amidite, 6-Fluorescein, IRDye 700, TYE 665, Alex Fluor 594, or ATTO TM 633 (NHS Ester). A quenching moiety may be Iowa Black RQ, Iowa Black FQ or IRDye QC-1 Quencher. A quenching moiety may quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

The generation of the detectable signal from the release of the detection moiety indicates that cleavage by the effector protein has occurred and that the sample contains the target nucleic acid. In some instances, the detection moiety comprises a fluorescent dye. Sometimes the detection moiety comprises a fluorescence resonance energy transfer (FRET) pair. In some instances, the detection moiety comprises an infrared (IR) dye. In some instances, the detection moiety comprises an ultraviolet (UV) dye. Alternatively, or in combination, the detection moiety comprises a protein. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal may be a colorimetric signal or a signal visible by eye. In some instances, the detectable signal may be fluorescent, electrical, chemical, electrochemical, or magnetic. In some instances, the first detection signal may be generated by binding of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some instances, the detectable signal may be generated directly by the cleavage event. Alternatively, or in combination, the detectable signal may be generated indirectly by the signal event. Sometimes the detectable signal is not a fluorescent signal. In some instances, the detectable signal may be a colorimetric or color-based signal. In some instances, the detected target nucleic acid may be identified based on its spatial location on the detection region of the support medium. In some instances, the second detectable signal may be generated in a spatially distinct location than the first generated signal.

In some instances, the reporter nucleic acid is a single-stranded nucleic acid sequence comprising ribonucleotides. The nucleic acid of a reporter may be a single-stranded nucleic acid sequence comprising at least one ribonucleotide. In some instances, the nucleic acid of a reporter is a single-stranded nucleic acid comprising at least one ribonucleotide residue at an internal position that functions as a cleavage site. In some instances, the nucleic acid of a reporter comprises at least 2, at least 3, at least 4, at least 5, at least 6, at least 7, at least 8, at least 9, or at least 10 ribonucleotide residues at an internal position. In some instances, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some instances, the nucleic acid of a reporter has only ribonucleotide residues. In some instances, the nucleic acid of a reporter has only deoxyribonucleotide residues. In some instances, the nucleic acid comprises nucleotides resistant to cleavage by the effector protein described herein. In some instances, the nucleic acid of a reporter comprises synthetic nucleotides. In some instances, the nucleic acid of a reporter comprises at least one ribonucleotide residue and at least one non-ribonucleotide residue.

In some instances, the nucleic acid of a reporter comprises at least one uracil ribonucleotide. In some instances, the nucleic acid of a reporter comprises at least two uracil ribonucleotides. Sometimes the nucleic acid of a reporter has only uracil ribonucleotides. In some instances, the nucleic acid of a reporter comprises at least one adenine ribonucleotide. In some instances, the nucleic acid of a reporter comprises at least two adenine ribonucleotide. In some instances, the nucleic acid of a reporter has only adenine ribonucleotides. In some instances, the nucleic acid of a reporter comprises at least one cytosine ribonucleotide. In some instances, the nucleic acid of a reporter comprises at least two cytosine ribonucleotide. In some instances, the nucleic acid of a reporter comprises at least one guanine ribonucleotide. In some instances, the nucleic acid of a reporter comprises at least two guanine ribonucleotide. In some instances, a nucleic acid of a reporter comprises a single unmodified ribonucleotide. In some instances, a nucleic acid of a reporter comprises only unmodified deoxyribonucleotides.

In some instances, the nucleic acid of a reporter is 5 to 20, 5 to 15, 5 to 10, 7 to 20, 7 to 15, or 7 to 10 nucleotides in length. In some instances, the nucleic acid of a reporter is 3 to 20, 4 to 10, 5 to 10, or 5 to 8 nucleotides in length. In some instances, the nucleic acid of a reporter is 5 to 12 nucleotides in length. In some instances, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some instances, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some instances, systems comprise a plurality of reporters. The plurality of reporters may comprise a plurality of signals. In some instances, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some instances, there are 2 to 50, 3 to 40, 4 to 30, 5 to 20, or 6 to 10 different reporters.

In some instances, systems comprise an effector protein and a reporter nucleic acid configured to undergo trans cleavage by the effector protein. Trans cleavage of the reporter may generate a signal from reporter or alter a signal from the reporter. In some instances, the signal is an optical signal, such as a fluorescence signal or absorbance band. Trans cleavage of the reporter may alter the wavelength, intensity, or polarization of the optical signal. For example, the reporter may comprise a fluorophore and a quencher, such that trans cleavage of the reporter separates the fluorophore and the quencher thereby increasing a fluorescence signal from the fluorophore. Herein, detection of reporter cleavage to determine the presence of a target nucleic acid sequence may be referred to as ‘DETECTR’. In some instances described herein is a method of assaying for a target nucleic acid in a sample comprising contacting the target nucleic acid with an effector protein, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of an effector protein (e.g., an effector protein as disclosed herein) may be inhibited. This is because the activated effector proteins collaterally cleave any nucleic acids. If total nucleic acids are present in large amounts, they may outcompete reporters for the effector proteins. In some instances, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some instances, the sample comprises amplified target nucleic acid. In some instances, the sample comprises an unamplified target nucleic acid. In some instances, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some instances, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

### Amplification Reagents/Components

In some instances, systems described herein comprise a reagent or component for amplifying a nucleic acid. Non-limiting examples of reagents for amplifying a nucleic acid include polymerases, primers, and nucleotides. In some instances, systems comprise reagents for nucleic acid amplification of a target nucleic acid in a sample. Nucleic acid amplification of the target nucleic acid may improve at least one of sensitivity, specificity, or accuracy of the assay in detecting the target nucleic acid. In some instances, nucleic acid amplification is isothermal nucleic acid amplification, providing for the use of the system or system in remote regions or low resource settings without specialized equipment for amplification. In some instances, amplification of the target nucleic acid increases the concentration of the target nucleic acid in the sample relative to the concentration of nucleic acids that do not correspond to the target nucleic acid.

The reagents for nucleic acid amplification may comprise a recombinase, an oligonucleotide primer, a single-stranded DNA binding (SSB) protein, a polymerase, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some instances, systems comprise a PCR tube, a PCR well or a PCR plate. The wells of the PCR plate may be pre-aliquoted with the reagent for amplifying a nucleic acid, as well as a guide nucleic acid, an effector protein, a multimeric complex, or any combination thereof. The wells of the PCR plate may be pre-aliquoted with a guide nucleic acid targeting a target sequence, an effector protein capable of being activated when complexed with the guide nucleic acid and the target sequence, and at least one population of a single stranded reporter nucleic acid comprising a detection moiety. A user may thus add the biological sample of interest to a well of the pre-aliquoted PCR plate and measure for the detectable signal with a fluorescent light reader or a visible light reader.

In some instances, systems comprise a PCR plate; a guide nucleic acid targeting a target sequence; an effector protein capable of being activated when complexed with the guide nucleic acid and the target sequence; and a single stranded reporter nucleic acid comprising a detection moiety, wherein the reporter nucleic acid is capable of being cleaved by the activated nuclease, thereby generating a detectable signal.

In some instances, systems comprise a support medium; a guide nucleic acid targeting a target sequence; and an effector protein capable of being activated when complexed with the guide nucleic acid and the target sequence. In some instances, nucleic acid amplification is performed in a nucleic acid amplification region on the support medium. Alternatively, or in combination, the nucleic acid amplification is performed in a reagent chamber, and the resulting sample is applied to the support medium.

In some instances, a system for modifying a target nucleic acid comprises a PCR plate; a guide nucleic acid targeting a target sequence; and an effector protein capable of being activated when complexed with the guide nucleic acid and the target sequence. The wells of the PCR plate may be pre-aliquoted with the guide nucleic acid targeting a target sequence, and an effector protein capable of being activated when complexed with the guide nucleic acid and the target sequence. A user may thus add the biological sample of interest to a well of the pre-aliquoted PCR plate.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. Sometimes, the nucleic acid amplification is performed for 1 to 60, 5 to 55, 10 to 50, 15 to 45, 20 to 40, or 25 to 35 minutes. Sometimes, the nucleic acid amplification reaction is performed at a temperature of around 20-45° C. In some instances, the nucleic acid amplification reaction is performed at a temperature no greater than 20° C., 25° C., 30° C., 35° C., 37° C., 40° C., 45° C., or any value 20° C. to 45° C. In some instances, the nucleic acid amplification reaction is performed at a temperature of at least 20° C., 25° C., 30° C., 35° C., 37° C., 40° C., or 45° C., or any value 20° C. to 45° C. In some instances, the nucleic acid amplification reaction is performed at a temperature of 20° C. to 45° C., 25° C. to 40° C., 30° C. to 40° C., or 35° C. to 40° C.

Often, systems comprise primers for amplifying a target nucleic acid to produce an amplification product comprising the target nucleic acid and a PAM. For instance, at least one of the primers may comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid sequence, methods of assaying for a target nucleic acid that lacks a PAM by amplifying the target nucleic acid sequence to introduce a PAM, and compositions used in introducing a PAM via amplification into the target nucleic acid sequence.

### Additional System Components

In some instances, systems include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of use.

A system may include labels listing contents and/or instructions for use, or package inserts with instructions for use. A set of instructions will also typically be included. In one embodiment, a label is on or associated with the container. In some instances, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

In some instances, systems comprise a solid support. An RNP or effector protein may be attached to a solid support. The solid support may be an electrode or a bead. The bead may be a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### Certain System Conditions

In some instances, systems and methods are employed under certain conditions that enhance an activity of the effector protein relative to alternative conditions, as measured by a detectable signal released from cleavage of a reporter in the presence of the target nucleic acid. The detectable signal may be generated at about the rate of trans cleavage of a reporter nucleic acid. In some instances, the reporter nucleic acid is a homopolymeric reporter nucleic acid comprising 5 to 20 consecutive adenines, 5 to 20 consecutive thymines, 5 to 20 consecutive cytosines, or 5 to 20 consecutive guanines. In some instances, the reporter is an RNA-FQ reporter.

In some instances, effector proteins disclosed herein recognize, bind, or are activated by, different target nucleic acids having different sequences, but are active toward the same reporter nucleic acid, allowing for facile multiplexing in a single assay having a single ssRNA-FQ reporter.

In some instances, systems are employed under certain conditions that enhance trans cleavage activity of an effector protein. In some instances, under certain conditions, transcolatteral cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some instances, systems and methods are employed under certain conditions that enhance cis-cleavage activity of the effector protein.

Certain conditions that may enhance the activity of an effector protein include a certain salt presence or salt concentration of the solution in which the activity occurs. For example, cis-cleavage activity of an effector protein may be inhibited or halted by a high salt concentration. The salt may be a sodium salt, a potassium salt, or a magnesium salt. In some instances, the salt is NaCl. In some instances, the salt is KNO₃. In some instances, the salt concentration is less than 150 mM, less than 125 mM, less than 100 mM, less than 75 mM, less than 50 mM, or less than 25 mM.

Certain conditions that may enhance the activity of an effector protein includes the pH of a solution in which the activity. For example, increasing pH may enhance trans cleavage activity. For example, the rate of trans cleavage activity may increase with increase in pH up to pH 9. In some instances, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some instances, the pH is 7 to 7.5, 7.5 to 8, 8 to 8.5, 8.5 to 9, or 7 to 8.5. In some instances, the pH is less than 7. In some instances, the pH is greater than 7.

Certain conditions that may enhance the activity of an effector protein includes the temperature at which the activity is performed. In some instances, the temperature is about 25° C. to about 50° C. In some instances, the temperature is about 20° C. to about 40° C., about 30° C. to about 50° C., or about 40° C. to about 60° C. In some instances, the temperature is about 25° C., about 30° C., about 35° C., about 40° C., about 45° C., or about 50° C.

In some instances, a final concentration an effector protein in a buffer of a system is 1 pM to 1 nM, 1 pM to 10 pM, 10 pM to 100 pM, 100 pM to 1 nM, 1 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1000 nM. The final concentration of the sgRNA complementary to the target nucleic acid may be 1 pM to 1 nM, 1 pM to 10 pM, 10 pM to 100 pM, 100 pM to 1 nM, 1 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1000 nM. The concentration of the ssDNA-FQ reporter may be 1 pM to 1 nM, 1 pM to 10 pM, 10 pM to 100 pM, 100 pM to 1 nM, 1 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1000 nM.

In some instances, systems comprise an excess volume of solution comprising the guide nucleic acid, the effector protein and the reporter, which contacts a smaller volume comprising a sample with a target nucleic acid. The smaller volume comprising the sample may be unlysed sample, lysed sample, or lysed sample which has undergone any combination of reverse transcription, amplification, and in vitro transcription. The presence of various reagents, (such as buffer, magnesium sulfate, salts, the pH, a reducing agent, primers, dNTPs, NTPs, cellular lysates, non-target nucleic acids, primers, or other components), in a crude, non-lysed sample, a lysed sample, or a lysed and amplified sample, may inhibit the ability of the effector protein to become activated or to find and cleave the nucleic acid of the reporter. This may be due to nucleic acids that are not the reporter outcompeting the nucleic acid of the reporter, for the effector protein. Alternatively, various reagents in the sample may simply inhibit the activity of the effector protein. Thus, the compositions and methods provided herein for contacting an excess volume comprising the engineered guide nucleic acid, the effector protein, and the reporter to a smaller volume comprising the sample with the target nucleic acid of interest provides for superior detection of the target nucleic acid by ensuring that the effector protein is able to find and cleaves the nucleic acid of the reporter. In some instances, the volume comprising the guide nucleic acid, the effector protein, and the reporter (may be referred to as “a second volume”) is 4-fold greater than a volume comprising the sample (may be referred to as “a first volume”). In some instances, the volume comprising the guide nucleic acid, the effector protein, and the reporter (may be referred to as “a second volume”) is at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold greater than a volume comprising the sample (may be referred to as “a first volume”). In some instances, the volume comprising the sample is at least 0.5 μL, at least 1 μL, at least at least 1 μL, at least 2 μL, at least 3 μL, at least 4 μL, at least 5 μL, at least 6 μL, at least 7 μL, at least 8 μL, at least 9 μL, at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 25 μL, at least 30 μL, at least 35 μL, at least 40 μL, at least 45 μL, at least 50 μL, at least 55 μL, at least 60 μL, at least 65 μL, at least 70 μL, at least 75 μL, at least 80 μL, at least 85 μL, at least 90 μL, at least 95 μL, at least 100 μL, 0.5 μL to 5 μL μL, 5 μL to 10 μL, 10 μL to 15 μL, 15 μL to 20 μL, 20 μL to 25 μL, 25 μL to 30 μL, 30 μL to 35 μL, 35 μL to 40 μL, 40 μL to 45 μL, 45 μL to 50 μL, 10 μL to 20 μL, 5 μL to 20 μL, 1 μL to 40 μL, 2 μL to 10 μL, or 1 μL to 10 μL. In some instances, the volume comprising the effector protein, the guide nucleic acid, and the reporter is at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 21 μL, at least 22 μL, at least 23 μL, at least 24 μL, at least 25 μL, at least 26 μL, at least 27 μL, at least 28 μL, at least 29 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 350 μL, at least 400 μL, at least 450 μL, at least 500 μL, 10 μL to 15 μL μL, 15 μL to 20 μL, 20 μL to 25 μL, 25 μL to 30 μL, 30 μL to 35 μL, 35 μL to 40 μL, 40 μL to 45 μL, 45 μL to 50 μL, 50 μL to 55 μL, 55 μL to 60 μL, 60 μL to 65 μL, 65 μL to 70 μL, 70 μL to 75 μL, 75 μL to 80 μL, 80 μL to 85 μL, 85 μL to 90 μL, 90 μL to 95 μL, 95 μL to 100 μL, 100 μL to 150 μL, 150 μL to 200 μL, 200 μL to 250 μL, 250 μL to 300 μL, 300 μL to 350 μL, 350 μL to 400 μL, 400 μL to 450 μL, 450 μL to 500 μL, 10 μL to 20 μL, 10 μL to 30 μL, 25 μL to 35 μL, 10 μL to 40 μL, 20 μL to 50 μL, 18 μL to 28 μL, or 17 μL to 22 μL.

In some instances, systems comprise an effector protein that nicks a target nucleic acid, thereby producing a nicked product. In some instances, systems cleave a target nucleic acid, thereby producing a linearized product. In some instances, systems produce at least 50%, at least 55%, at least 60%, at least 65%, at least 70%, at least 75%, at least 80%, at least 85%, at least 90 or at least 95% of a maximum amount of nicked product within 1 minute, where the maximum amount of nicked product is the maximum amount detected within a 60 minute period from when the target nucleic acid is mixed with the effector protein or the multimeric complex thereof. In some instances, systems produce at least 50%, at least 55%, at least 60%, at least 65%, at least 70%, at least 75%, at least 80%, at least 85%, at least 90 or at least 95% of a maximum amount of linearized product within 1 minute, where the maximum amount of linearized product is the maximum amount detected within a 60-minute period from when the target nucleic acid is mixed with the effector protein. In some instances, at least 80% of the maximum amount of linearized product is produced within 1 minute. In some instances, at least 90% of the maximum amount of linearized product is produced within 1 minute.

### X. Target Nucleic Acids and Samples

Described herein are compositions, systems and methods for modifying or detecting a target nucleic acid, wherein the target nucleic acid is a gene, a portion thereof, a transcript thereof. In some embodiments, the target nucleic acid is a reverse transcript (e.g. a cDNA) of an mRNA transcribed from the gene, or an amplicon thereof. acid. In some embodiments, the target nucleic acid is a single stranded nucleic acid. Alternatively, or in combination, the target nucleic acid is a double stranded nucleic acid and is prepared into single stranded nucleic acids before or upon contacting the reagents. In some embodiments, the target nucleic acid is a double stranded nucleic acid. In some embodiments, the double stranded nucleic acid is DNA. The target nucleic acid may be a RNA. The target nucleic acids include but are not limited to mRNA, rRNA, tRNA, non-coding RNA, long non-coding RNA, and microRNA (miRNA). In some embodiments, the target nucleic acid is complementary DNA (cDNA) synthesized from a single-stranded RNA template in a reaction catalyzed by a reverse transcriptase. In some embodiments, the target nucleic acid is single-stranded RNA (ssRNA) or mRNA. In some embodiments, the target nucleic acid is from a virus, a parasite, or a bacterium described herein. As another non-limiting example, the target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides).

In certain embodiments, the target nucleic acid is a double stranded nucleic acid comprising a target strand and a non-target strand, wherein the target strand comprises a target sequence. In some embodiments, where a target strand comprises a target sequence, at least a portion of the engineered guide nucleic acid is complementary to the target sequence on the target strand. In some embodiments, where the target nucleic acid is a double stranded nucleic acid comprising a target strand and a non-target strand, and wherein the target strand comprises a target sequence, at least a portion of the engineered guide nucleic acid is complementary to the target sequence on the target strand. In some embodiments, a target nucleic acid comprises a PAM as described herein that is located on the non-target strand. Such a PAM described herein, in some embodiments, is adjacent (e.g., within 1, 2, 3, 4 or 5 nucleotides) to the 5′ end of the target sequence on the non-target strand of the double stranded DNA molecule. In certain embodiments, such a PAM described herein is directly adjacent to the 5′ end of a target sequence on the non-target strand of the double stranded DNA molecule.

In some embodiments, an effector protein described herein, or a multimeric complex thereof recognizes a PAM on a target nucleic acid. In some embodiments, multiple effector proteins of the multimeric complex recognize a PAM on a target nucleic acid. In some embodiments, only one effector protein of the multimeric complex recognizes a PAM on a target nucleic acid. In some embodiments, the PAM is 3′ to the spacer region of the crRNA. In some embodiments, the PAM is directly 3′ to the spacer region of the crRNA.

An effector protein of the present disclosure, a dimer thereof, or a multimeric complex thereof may cleave or nick a target nucleic acid within or near a protospacer adjacent motif (PAM) sequence of the target nucleic acid. In some embodiments, cleavage occurs within 1, 2, 3, 4, 5, 6, 7, 8, 9 or 10 nucleotides of a 5′ or 3′ terminus of a PAM sequence. A target nucleic acid may comprise a PAM sequence adjacent to a sequence that is complementary to a guide nucleic acid spacer region.

In some embodiments, the target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides.

In some embodiments, the target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are set forth in TABLE 4. Nucleic acid sequences of target nucleic acids and/or corresponding genes are readily available in public databases as known and used in the art. In some embodiments, the target nucleic acid is selected from TABLE 4.

TABLE 4
EXEMPLARY TARGETS
Exemplary targets

AAVS1, ABCA4, ABCB11, ABCC8, ABCD1, ABCG5, ABCG8, ACAD9, ACADM, ACADVL, ACAT1,
ACOX1, ACSF3, ADA, ADAMTS2, ADGRG1, AGA, AGL, AGPS, AGXT, AHI1, AIRE, ALDH3A2,
ALDOB, ALG6, ALK, ALKBH5, ALMS1, ALPL, AMRC9, AMT, ANAPC10, ANAPC11, ANGPTL3, APC,
Apo(a), APOCIII, APOEε4, APOL1, APP, AQP2, AR, ARFRP1, ARG1, ARH, ARL13B, ARL6, ARSA,
ARSB, ASL, ASNS, ASPA, ASS1, ATM, ATP6V1B1, ATP7A, ATP7B, ATRX, ATXN1, ATXN10, ATXN2,
ATXN3, ATXN7, ATXN8OS, AXIN1, AXIN2, B2M, BACE-1, BAK1, BAP1, BARD1, BAX2, BBS1,
BBS10, BBS12, BBS2, BCKDHA, BCKDHB, BCL2L2, BCS1L, BEST1, Betaglobin gene, BLM, BMPR1A,
BRAF, BRAFV600E, BRCA1, BRCA2, BRIP1, BSND, C9orf72, CA4, CACNA1A, CAPN3, CASR, CBS,
CCNB1 CC2D2A, CCR5, CD1, CD2, CD3, CD3D, CD3Z, CD4, CD5, CD6, CD7, CD8A, CD8B, CD9,
CD14, CD18, CD19, CD21, CD22, CD23, CD27, CD28, CD30, CD33, CD34, CD36, CD38, CD40,
CD40L, CD44, CD46, CD47, CD48, CD52, CD55, CD57, CD58, CD59, CD68, CD69, CD72, CD73,
CD74, CD79A, CD80, CD81, CD83, CD84, CD86, CD90, CD93, CD96, CD99, CD100, CD123, CD160,
CD163, CD164, CD164L2, CD166, CD200, CD204, CD207, CD209, CD226, CD244, CD247, CD274,
CD276, CD300, CD320, CDC73, CDH1, CDH23, CDK11, CDK4, CDKN1A, CDKN1B, CDKN1C,
CDKN2A, CDKN2B, CEBPA, CELA3B, CEP290, CERKL, CFB, CFTR, CHCHD10, CHEK2, CHM, CHRNE,
CIITA, CLN3, CLN5, CLN6, CLN8, CLRN1, CLTA, CNBP, CNGB1, CNGB3, COL1A1, COL1A2,
COL27A1, COL4A3, COL4A4, COL4A5, COL7A1, CPS1, CPT1A, CPT2, CRB1, CREBBP, CRX, CRYAA,
CTNNA1, CTNNB1, CTNND2, CTNS, CTSK, CXCL12, CYBA, CYBB, CYP11B1, CYP11B2, CYP17A1,
CYP19A1, CYP27A1, DBT, DCC, DCLRE1C, DERL2, DFNA36, DFNB31, DGAT2, DHCR7, DHDDS,
DICER1, DIS3L2, DLD, DMD, DMPK, DNAH5, DNAI1, DNAI2, DNM2, DNMT1, DPC4, DYSF, EDA,
EDN3, EDNRB, EGFR, EIF2B5, EMC2, EMC3, EMD, EMX1, EN1, EPCAM, ERCC6, ERCC8, ESCO2,
ETFA, ETFDH, ETHE1, EVC, EVC2, EYS, F5, F9, FXI, FAH, FAM161A, FANCA, FANCB, FANCC,
FANCD1, FANCD2, FANCE, FANCF, FANCG, FANCI, FANCJ, FANCL, FANCM, FANCN, FANCP,
FANCS, FBN1, FGF14, FGFR2, FGFR3, FGA, FGB, FGG, FH, FHL1, FIX, FKRP, FKTN, FLCN, FMR1,
FOXP3, FSCN2, FUS, FUT8, FVIII, FXII, FXN, G6PC, GAA, GALC, GALK1, GALT, GAMT, GATA2,
GATA-4, GBA, GBE1, GCDH, GCGR, GDNF, GFAP, GFM1, GHR, GJB1, GJB2, GLA, GLB1, GLDC,
GLE1, GNE, GNPTAB, GNPTG, GNS, GPC3, GPR98, GREM1, GRHPR, GRIN2B, H2AFX, H2AX,
HADHA, HAX1, HBA1, HBA2, HBB, HER2, HEXA, HEXB, HFE, HGSNAT, HLCS, HMGCL, HOGA1,
HOXB13, HPRPF3, HPRT1, HPS1, HPS3, HRAS, HRD1, HSD17B4, HSD3B2, HTT, HUS1, HYAL1,
HYLS1, IDS, IDUA, IFITM5, IKBKAP, IL2RG, IL7R, IMPDH1, INPP5E, IRF4, ITGB2, ITPR1, IVD, JAG1,
JAK1, JAK3, KCNC3, KCND3, KCNJ11, KLHL7, KRAS, LAMA2, LAMA3, LAMB3, LAMC2, LCA5, LDLR,
LDLRAP1, LHX3, LIFR, LIPA, LMNA, LOR, LOXHD1, LPL, LRAT, LRP6, LRPPRC, LRRK2, MADR2,
MAN2B1, MAPT, MAX, MCM6, MCOLN1, MECP2, MED17, MEFV, MEN1, MERTK, MESP2, MET,
METex14, MFN2, MFSD8, MIA3, MITF, MKL2, MKS1, MLC1, MLH1, MLH3, MMAA, MMAB,
MMACHC, MMADHC, MMD, MPI, MPL, MPV17, MSH2, MSH3, MSH6, MTHFD1L, MTHFR,
MTM1, MTRR, MTTP, MUT, MUTYH, MYC, MYH7, MYO7A, NAGLU, NAGS, NBN, NDRG1,
NDUFAF5, NDUFS6, NEB, NF1, NF2, NKX2-5, NOG, NOTCH1, NOTCH2, NPC1, NPC2, NPHP1,
NPHS1, NPHS2, NRAS, NR2E3, NTHL1, NTRK, NTRK1, OAT, OCT4, OFD1, OPA3, OTC, PAH, PALB2,
PAQR8, PAX3, PC, PCCA, PCCB, PCDH15, PCSK9, PD1, PDCD1, PDE6B, PDGFRA, PDHA1, PDHB,
PEX1, PEX10, PEX12, PEX13, PEX14, PEX16, PEX19, PEX2, PEX26, PEX3, PEX5, PEX6, PEX7, PFKM,
PHGDH, PHOX2B, PKD1, PKD2, PKHD1, PKK, PLEKHG4, PMM2, PMP22, PMS1, PMS2, PNPLA3,
POLD1, POLE, POMGNT1, POT1, POU5F1, PPM1A, PPP2R2B, PPT1, PRCD, PRKAG2, PRKAR1A,
PRKCG, PRNP, PROM1, PROP1, PRPF31, PRPF8, PRPH2, PRPS1, PSAP, PSD95, PSEN1, PSEN2,
PSRC1, PTCH1, PTEN, PTS, PUS1, PYGM, RAB23, RAD50, RAD51C, RAD51D, RAG1, RAG2, RAPSN,
RARS2, RB1, RDH12, RECQL4, RET, RHO, RICTOR, RMRP, ROS1, RP1, RP2, RPE65, RPGR,
RPGRIP1L, RPL32P3, RS1, RTCA, RTEL1, RUNX1, SACS, SAMHD1, SCN1A, SCN2A, SDHA, SDHAF2,
SDHB, SDHC, SDHD, SEL1L, SEPSECS, SERPINA1, SERPING1, SGCA, SGCB, SGCG, SGSH, SIRT1,
SLC12A3, SLC12A6, SLC17A5, SLC22A5, SLC25A13, SLC25A15, SLC26A2, SLC26A4, SLC35A3,
SLC35B4 SLC37A4, SLC39A4, SLC4A11, SLC6A8, SLC7A7, SMAD3, SMAD4, SMARCA4, SMARCAL1,
SMARCB1, SMARCE1, SMN1, SMPD1, SNAI2, SNCA, SNRNP200, SOD1, SOX10, SPARA7, SPTBN2,
STAR, STAT3, STK11, SUFU, SUMF1, SYNE1, SYNE2, SYS1, TARDBP, TAT, TBK1, TBP, TCIRG1,
TCTN3, TECPR2, TERC, TERT, TFR2, TGFBR2, TGM1, TH, TLE3, TMEM127, TMEM138, TMEM216,
TMEM43, TMEM67, TMPRSS6, TOP1, TOPORS, TP53, TPP1, TRAC, TRMU, TSC1, TSC2, TSFM,
TSPAN14, TTBK2, TTC8, TTPA, TTR, TULP1, TYMP, UBE2G2, UBE2J1, UBE3A, USH1C, USH1G,
USH2A, VEGF, VHL, VPS13A, VPS13B, VPS35, VPS45, VRK1, VSX2, VWF, WAS, WDR19, WDR48,
WNT10A, WRN, WS2B, WS2C, WT1, XPA, XPC, XPF, XRCC3, YAP1, ZAC1, ZEB1, ZFYVE26, and
ZNF423

In some embodiments, the target nucleic acid comprises a target locus. In certain embodiments, the target nucleic acid comprises more than one target loci. In some embodiments, the target nucleic acid comprises two target loci. Accordingly, in some embodiments, the target nucleic acid can comprise one or more target sequences.

In some embodiments, the one or more target sequence is within any one of the genes set forth in Table. In some embodiments, the target sequence is within an exon of any one of the genes set forth in Table. In some embodiments, then target sequence covers the junction of two exons. In some embodiments, the target sequence is located within about 1 to about 300 nucleotides, about 10 to about 250, about 20 to about 200, about 30 to about 150, about 40 to about 100, or about 50 nucleotides of the 5′ untranslated region (UTR). In some embodiments, the target sequence is located within about 1 to about 300 nucleotides, about 10 to about 250, about 20 to about 200, about 30 to about 150, about 40 to about 100, or about 50 nucleotides of the 3′ UTR.

In some embodiments, the target sequence is at least partially within a targeted exon within any one of the genes set forth in Table. A targeted exon can mean any portion within, contiguous with, or adjacent to a specified exon of interest can be targeted by the compositions, systems, and methods described herein. In some embodiments, one or more of the exons are targeted. In some embodiments, one or more of exons of any one the genes set forth in Table are targeted.

In some embodiments, the start of an exon is referred to interchangeably herein as the 5′ end of an exon. In certain embodiments, the 5′ region of an exon comprises a sequence about 1 to about 300 nucleotides adjacent to the 5′ end of an exon when moving upstream in the 5′ direction, or a sequence about 1 to about 300 nucleotides adjacent to the 5′ end of an exon when moving downstream in the 3′ direction, or both.

In some embodiments, the end of an exon is referred to interchangeably herein as the 3′ end of an exon. In certain embodiments, the 3′ region of an exon comprises a sequence about 1 to about 300 nucleotides adjacent to the 3′ end of an exon when moving upstream in the 5′ direction, or a sequence about 1 to about 300 nucleotides adjacent to the 3′ end of an exon when moving downstream in the 3′ direction, or both.

Nucleic acids, such as DNA and pre-mRNA, can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3′SS can refer to the last two positions at the 3′ end of an intron. Alternatively, a 5′ SS can refer to the 5′ end of an exon and a 3′SS can refer to the 3′ end of an exon. In certain embodiments, nucleic acids can contain one or more elements that act as a signal during transcription, splicing, and/or translation. In certain embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs).

In some embodiments, a target sequence that a guide nucleic acid binds is at least partially within a targeted exon within any one of the genes set forth in TABLE 4, and wherein at least a portion of the target nucleic acid is within a sequence about 1 to about 300 nucleotides adjacent to: the start of a targeted exon, the end of a targeted exon, or both. In some embodiments, at least a portion of the target sequence that a guide nucleic acid binds can comprise a sequence about 1 to about 300 nucleotides, about 10 to about 250, about 20 to about 200, about 30 to about 150, about 40 to about 100, or about 50 nucleotides adjacent to: the start of a targeted exon, the end of a targeted exon, or both.

In some embodiments, at least a portion of the target nucleic acid that a guide nucleic acid binds is within a sequence about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 or more nucleotides adjacent to: the start of a targeted exon, the end of a targeted exon, or both.

In some embodiments, a target sequence that a guide nucleic acid binds is at least partially within a targeted exon within any one of the genes set forth in TABLE 4, and wherein at least a portion of the target nucleic acid is within a sequence about 1 to about 300 nucleotides adjacent to: the start of a targeted exon, the end of a targeted exon, or both. In some embodiments, at least a portion of the target sequence that a guide nucleic acid binds can comprise a sequence about 1 to about 300 nucleotides, about 10 to about 250, about 20 to about 200, about 30 to about 150, about 40 to about 100, or about 50 nucleotides adjacent to: one or more signaling element comprising a 5′SS, a 3′SS, a premature stop codon, U1 binding sequence, U2 binding sequence, a BS, a PYT, ESE, an ISE, an ESS, an ISS, more than one of the foregoing, or any combination thereof.

In certain embodiments, at least a portion of the target nucleic acid that a guide nucleic acid binds is within a sequence about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 or more nucleotides adjacent to: one or more signaling element comprising a 5′SS, a 3′SS, a premature stop codon, U1 binding sequence, U2 binding sequence, a BS, a PYT, ESE, an ISE, an ESS, an ISS, more than one of the foregoing, or any combination thereof.

Further description of editing or detecting a target nucleic acid in the foregoing genes can be found in more detail in Kim et al., “Enhancement of target specificity of CRISPR-Cas12a by using a chimeric DNA-RNA guide”, Nucleic Acids Res. 2020 Sep. 4; 48(15):8601-8616; Wang et al., “Specificity profiling of CRISPR system reveals greatly enhanced off-target gene editing”, Scientific Reports volume 10, Article number: 2269 (2020); Tuladhar et al., “CRISPR-Cas9-based mutagenesis frequently provokes on-target mRNA misregulation”, Nature Communications volume 10, Article number: 4056 (2019); Dong et al., “Genome-Wide Off-Target Analysis in CRISPR-Cas9 Modified Mice and Their Offspring”, G3, Volume 9, Issue 11, 1 Nov. 2019, Pages 3645-3651; Winter et al., “Genome-wide CRISPR screen reveals novel host factors required for Staphylococcus aureus a-hemolysin-mediated toxicity”, Scientific Reports volume 6, Article number: 24242 (2016); and Ma et al., “A CRISPR-Based Screen Identifies Genes Essential for West-Nile-Virus-Induced Cell Death”, Cell Rep. 2015 Jul. 28; 12(4):673-83, which are hereby incorporated by reference in their entirety.

In some embodiments, the target nucleic acid is in a cell. In general, the cell is a human cell. In some embodiments, the human cell is a: muscle cell, cardiac cell, visceral cell, cardiac muscle cell, smooth muscle cell, cardiomyocyte, nodal cardiac muscle cell, smooth muscle cell, visceral muscle cell, skeletal muscle cell, myocyte, red (or slow) skeletal muscle cell, white (fast) skeletal muscle cell, intermediate skeletal muscle, muscle satellite cell, muscle stem cell, myoblast, muscle progenitor cell, induced pluripotent stem cell (iPS), or a cell derived from an iPS cell, modified to have its gene edited and differentiated into myoblasts, muscle progenitor cells, muscle satellite cells, muscle stem cells, skeletal muscle cells, cardiac muscle cells or smooth muscle cells.

An effector protein-guide nucleic acid complex may comprise high selectivity for a target sequence. In some embodiments, a ribonucleoprotein may comprise a selectivity of at least 200:1, 100:1, 50:1, 20:1, 10:1, or 5:1 for a target nucleic acid over a single nucleotide variant of the target nucleic acid. In some embodiments, a ribonucleoprotein may comprise a selectivity of at least 5:1 for a target nucleic acid over a single nucleotide variant of the target nucleic acid. Leveraging Effector protein selectivity, some methods described herein may detect a target nucleic acid present in the sample in various concentrations or amounts as a target nucleic acid population. In some embodiments, the sample has at least 2 target nucleic acids. In some embodiments, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some embodiments, the sample comprises 1 to 10,000, 100 to 8000, 400 to 6000, 500 to 5000, 1000 to 4000, or 2000 to 3000 target nucleic acids. In some embodiments, the method detects target nucleic acid present at least at one copy per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

Often, the target nucleic acid may be from 0.05% to 20% of total nucleic acids in the sample. Sometimes, the target nucleic acid is 0.1% to 10% of the total nucleic acids in the sample. The target nucleic acid, in some embodiments, is 0.1% to 5% of the total nucleic acids in the sample. The target nucleic acid may also be 0.1% to 1% of the total nucleic acids in the sample. The target nucleic acid may be DNA or RNA. The target nucleic acid may be any amount less than 100% of the total nucleic acids in the sample. The target nucleic acid may be 100% of the total nucleic acids in the sample.

The target nucleic acid may be 0.05% to 20% of total nucleic acids in the sample. Sometimes, the target nucleic acid is 0.1% to 10% of the total nucleic acids in the sample. The target nucleic acid, in some embodiments, is 0.1% to 5% of the total nucleic acids in the sample. Often, a sample comprises the segment of the target nucleic acid and at least one nucleic acid comprising less than 100% sequence identity to the segment of the target nucleic acid but no less than 50% sequence identity to the segment of the target nucleic acid. For example, the segment of the target nucleic acid comprises a mutation as compared to at least one nucleic acid comprising less than 100% sequence identity to the segment of the target nucleic acid but no less than 50% sequence identity to the segment of the target nucleic acid. Often, the segment of the target nucleic acid comprises a single nucleotide mutation as compared to at least one nucleic acid comprising less than 100% sequence identity to the segment of the target nucleic acid but no less than 50% sequence identity to the segment of the target nucleic acid.

A target nucleic acid may be an amplified nucleic acid of interest. The nucleic acid of interest may be any nucleic acid disclosed herein or from any sample as disclosed herein. The nucleic acid of interest may be an RNA that is reverse transcribed before amplification. The nucleic acid of interest may be amplified then the amplicons may be transcribed into RNA.

In some embodiments, compositions described herein exhibit indiscriminate trans-cleavage of ssRNA, enabling their use for detection of RNA in samples. In some embodiments, target ssRNA are generated from many nucleic acid templates (RNA) in order to achieve cleavage of the FQ reporter in the DETECTR platform. Certain Effector proteins may be activated by ssRNA, upon which they may exhibit trans-cleavage of ssRNA and may, thereby, be used to cleave ssRNA FQ reporter molecules in the DETECTR system. These Effector proteins may target ssRNA present in the sample or ssRNA generated and/or amplified from any number of nucleic acid templates (RNA). Described herein are reagents comprising a single stranded reporter nucleic acid comprising a detection moiety, wherein the reporter nucleic acid (e.g., the ssDNA-FQ reporter described above) is capable of being cleaved by the Effector protein, upon generation and amplification of ssRNA from a nucleic acid template using the methods disclosed herein, thereby generating a first detectable signal.

In some embodiments, target nucleic acids comprise at least one nucleic acid comprising at least 50% sequence identity to the target nucleic acid or a portion thereof. Sometimes, the at least one nucleic acid comprises an amino acid sequence that is at least 60%, 70%, 75%, 80%, 85%, 90%, 91%, 92%, 93%, 94%, 95%, 96%, 97%, 98%, or 99% identical to an equal length portion of the target nucleic acid. Sometimes, the at least one nucleic acid comprises an amino acid sequence that is 100% identical to an equal length portion of the target nucleic acid. Sometimes, the amino acid sequence of the at least one nucleic acid is at least 60%, 70%, 75%, 80%, 85%, 90%, 91%, 92%, 93%, 94%, 95%, 96%, 97%, 98%, or 99% identical to the target nucleic acid. Sometimes, the target nucleic acid comprises an amino acid sequence that is less than 60%, 70%, 75%, 80%, 85%, 90%, 91%, 92%, 93%, 94%, 95%, 96%, 97%, 98%, or 99% identical to an equal length portion of the at least one nucleic acid.

In some embodiments, samples comprise a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM, 3 μM to 4 μM, 4 μM to 5 μM, 5 μM to 6 μM, 6 μM to 7 μM, 7 μM to 8 μM, 8 μM to 9 μM, 9 μM to 10 μM, 10 μM to 100 μM, 100 μM to 1 mM, 1 nM to 10 nM, 1 nM to 100 nM, 1 nM to 1 μM, 1 nM to 10 μM, 1 nM to 100 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 μM, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some embodiments, the sample comprises a target nucleic acid at a concentration of 20 nM to 200 μM, 50 nM to 100 μM, 200 nM to 50 μM, 500 nM to 20 μM, or 2 μM to 10 μM. In some embodiments, the target nucleic acid is not present in the sample.

In some embodiments, samples comprise fewer than 10 copies, fewer than 100 copies, fewer than 1000 copies, fewer than 10,000 copies, fewer than 100,000 copies, or fewer than 1,000,000 copies of a target nucleic acid. In some embodiments, the sample comprises 10 copies to 100 copies, 100 copies to 1000 copies, 1000 copies to 10,000 copies, 10,000 copies to 100,000 copies, 100,000 copies to 1,000,000 copies, 10 copies to 1000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid. In some embodiments, the sample comprises 10 copies to 500,000 copies, 200 copies to 200,000 copies, 500 copies to 100,000 copies, 1000 copies to 50,000 copies, 2000 copies to 20,000 copies, 3000 copies to 10,000 copies, or 4000 copies to 8000 copies. In some embodiments, the target nucleic acid is not present in the sample.

A number of target nucleic acid populations are consistent with the methods and compositions disclosed herein. Some methods described herein may detect two or more target nucleic acid populations present in the sample in various concentrations or amounts. In some embodiments, the sample has at least 2 target nucleic acid populations. In some embodiments, the sample has at least 3, 4, 5, 6, 7, 8, 9, 10, 20, 30, 40, or 50 target nucleic acid populations. In some embodiments, the sample has 3 to 50, 5 to 40, or 10 to 25 target nucleic acid populations. In some embodiments, the method detects target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations may be present at different concentrations or amounts in the sample.

In some embodiments, target nucleic acids may activate an effector protein to initiate sequence-independent cleavage of a nucleic acid-based reporter (e.g., a reporter comprising an RNA sequence, or a reporter comprising DNA and RNA). For example, an effector protein of the present disclosure is activated by a target nucleic acid to cleave reporters having an RNA (also referred to herein as an “RNA reporter”). Alternatively, an effector protein of the present disclosure is activated by a target nucleic acid to cleave reporters having an RNA. Alternatively, an effector protein of the present disclosure is activated by a target RNA to cleave reporters having an RNA (also referred to herein as a “RNA reporter”). The RNA reporter may comprise a single-stranded RNA labelled with a detection moiety or may be any RNA reporter as disclosed herein.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector protein system.

In some embodiments, the target nucleic acid is in a cell. In some embodiments, the cell is a single-cell eukaryotic organism; a plant cell an algal cell; a fungal cell; an animal cell; a cell an invertebrate animal; a cell a vertebrate animal such as fish, amphibian, reptile, bird, and mammal; or a cell a mammal such as a human, a non-human primate, an ungulate, a feline, a bovine, an ovine, and a caprine. In preferred embodiments, the cell is a eukaryotic cell. In preferred embodiments, the cell is a mammalian cell, a human cell, or a plant cell.

In some embodiments, the target nucleic acid comprises a nucleic acid sequence from a pathogen responsible for a disease. Non-limiting examples of pathogens are bacteria, a virus and a fungus. The target nucleic acid, in some embodiments, is a portion of a nucleic acid from a sexually transmitted infection or a contagious disease. In some embodiments, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to coronavirus (e.g., SARS-CoV-2); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some embodiments, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the target nucleic acid comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure may be used to target a viral nucleic acid sequence in a plant. An effector protein of the disclosure may cleave the viral nucleic acid. In some embodiments, the target nucleic acid comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some embodiments, is a portion of a nucleic acid from a virus or a bacterium or other agents responsible for a disease in the plant (e.g., a crop). In some embodiments, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that may be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

In some embodiments, compositions, systems, and methods described herein comprise a modified target nucleic acid which can describe a target nucleic acid wherein the target nucleic acid has undergone a modification, for example, after contact with an effector protein. In some embodiments, the modification is an alteration in the sequence of the target nucleic acid. In some embodiments, the modified target nucleic acid comprises an insertion, deletion, or replacement of one or more nucleotides compared to the unmodified target nucleic acid. In some embodiments, the modification is a mutation.

### Mutations

In some embodiments, target nucleic acids comprise a mutation. In some embodiments, a composition, system or method described herein can be used to modify a target nucleic acid comprising a mutation such that the mutation is modified to be a wild-type nucleotide or nucleotide sequence. In some embodiments, a composition, system or method described herein can be used to detect a target nucleic acid comprising a mutation. In some embodiments, a sequence comprising a mutation may be modified to a wildtype sequence with a composition, system or method described herein. In some embodiments, a sequence comprising a mutation may be detected with a composition, system or method described herein. The mutation may be a mutation of 1, 2, 3, 4, 5, 6, 7, 8, 9, 10, 11, 12, 13, 14, 15, 16, 17, 18, 19, 20 or more nucleotides. The mutation may comprise a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may comprise a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. Non-limiting examples of mutations are insertion-deletion (indel), single nucleotide polymorphism (SNP), and frameshift mutations. In some embodiments, guide nucleic acids described herein hybridize to a region of the target nucleic acid comprising the mutation. The mutation may be located in a non-coding region or a coding region of a gene.

A mutation may be in an open reading frame of a target nucleic acid. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

In some embodiments, a mutation comprises a point mutation or single nucleotide polymorphism (SNP), a chromosomal mutation, a copy number mutation, or any combination thereof. A point mutation optionally comprises a substitution, insertion, or deletion. In some embodiments, a mutation comprises a chromosomal mutation. A chromosomal mutations can comprise an inversion, a deletion, a duplication, or a translocation of one or more nucleotides. In some embodiments, a mutation comprises a copy number variation. A copy number variation can comprise a gene amplification or an expanding trinucleotide repeat. In some embodiments, guide nucleic acids described herein hybridize to a target sequence of a target nucleic acid comprising the mutation. In some embodiments, mutations are located in a non-coding region of a gene.

In some embodiments, target nucleic acids comprise a mutation, wherein the mutation is a SNP. The single nucleotide mutation or SNP may be associated with a phenotype of the sample or a phenotype of the organism from which the sample was taken. The SNP, in some embodiments, is associated with altered phenotype from wild type phenotype. In some embodiments, a single nucleotide mutation, SNP, or deletion described herein is associated with a disease, such as a genetic disease. The SNP may be a synonymous substitution or a nonsynonymous substitution. The nonsynonymous substitution may be a missense substitution or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a cancer cell.

In some embodiments, the target nucleic acid comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

In some embodiments, target nucleic acids comprise a mutation, wherein the mutation is a deletion of 1, 2, 3, 4, 5, 6, 7, 8, 9, 10, 11, 12, 13, 14, 15, 16, 17, 18, 19, 20 or more nucleotides. In some embodiments, a target nucleic acid comprises a mutation of 1, 2, 3, 4, 5, 6, 7, 8, 9, 10, 11, 12, 13, 14, 15, 16, 17, 18, 19, 20 or more nucleotides. The mutation may be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

### Certain Samples

Various sample types comprising a target nucleic acid of interest are consistent with the present disclosure. These samples may comprise a target nucleic acid for detection. In some instances, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample may be obtained to test for presence of a disease, cancer, genetic disorder, or any mutation of interest.

In some embodiments, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A tissue sample from a subject may be dissociated or liquified prior to application to detection system of the present disclosure. Non-limiting examples of environmental samples are soil, air, or water. In some embodiments, an environmental sample is taken as a swab from a surface of interest or taken directly from the surface of interest.

In some embodiments, the sample is a raw (unprocessed, unmodified) sample. Raw samples may be applied to a system for detecting or modifying a target nucleic acid, such as those described herein. In some embodiments, the sample is diluted with a buffer or a fluid or concentrated prior to its application to the system or be applied neat to the detection system. Sometimes, the sample contains no more 20 μl of buffer or fluid. The sample, in some embodiments, is contained in no more than 1, 5, 10, 15, 20, 25, 30, 35 40, 45, 50, 55, 60, 65, 70, 75, 80, 90, 100, 200, 300, 400, 500 μl, or any of value 1 μl to 500 μl, preferably 10 μL to 200 μL, or more preferably 50 μL to 100 μL of buffer or fluid. Sometimes, the sample is contained in more than 500 μl.

In some embodiments, the sample is taken from a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some embodiments, the sample is taken from nematodes, protozoans, helminths, or malarial parasites. In some embodiments, the sample comprises nucleic acids from a cell lysate from a eukaryotic cell, a mammalian cell, a human cell, a prokaryotic cell, or a plant cell. In some embodiments, the sample comprises nucleic acids expressed from a cell.

In some embodiments, samples are used for diagnosing a disease. In some embodiments the disease is cancer. The sample used for cancer testing may comprise at least one target nucleic acid that may bind to a guide nucleic acid of the reagents described herein. The target nucleic acid, in some embodiments, comprises a portion of a gene comprising a mutation associated with a disease, such as cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some embodiments, the assay may be used to detect “hotspots” in target nucleic acids that may be predictive of lung cancer. In some embodiments, the target nucleic acid comprises a portion of a nucleic acid that is associated with a blood fever. In some embodiments, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, system, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1. Any region of the aforementioned gene loci may be probed for a mutation or deletion using the compositions and methods disclosed herein. For example, in the EGFR gene locus, the compositions and methods for detection disclosed herein may be used to detect a single nucleotide polymorphism or a deletion.

In some embodiments, samples are used to diagnose a genetic disorder, also referred to as genetic disorder testing. The sample used for genetic disorder testing may comprise at least one target nucleic acid that may bind to a guide nucleic acid of the reagents described herein. In some embodiments, the genetic disorder is hemophilia, sickle cell anemia, β-thalassemia, Duchene muscular dystrophy, severe combined immunodeficiency, Huntington's disease, or cystic fibrosis. The target nucleic acid, in some embodiments, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some embodiments, the target nucleic acid is a nucleic acid from a genomic locus, a transcribed mRNA, or a reverse transcribed mRNA, a DNA amplicon of or a cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing may comprise at least one target nucleic acid that may bind to a guide nucleic acid of the reagents described herein. The target nucleic acid, in some embodiments, is a nucleic acid encoding a sequence associated with a phenotypic trait.

The sample used for genotyping testing may comprise at least one target nucleic acid that may bind to a guide nucleic acid of the reagents described herein. The target nucleic acid, in some embodiments, is a nucleic acid encoding a sequence associated with a genotype of interest.

The sample used for ancestral testing may comprise at least one target nucleic acid that may bind to a guide nucleic acid of the reagents described herein. The target nucleic acid, in some embodiments, is a nucleic acid encoding a sequence associated with a geographic region of origin or ethnic group.

The sample may be used for identifying a disease status. For example, a sample is any sample described herein, and is obtained from a subject for use in identifying a disease status of a subject. The disease may be a cancer or genetic disorder. Sometimes, a method comprises obtaining a serum sample from a subject; and identifying a disease status of the subject. Often, the disease status is prostate disease status, but the status of any disease may be assessed.

In some instances, compositions, systems and methods disclosed herein may be employed for detecting the genotype or phenotype of a cell or organism by contacting a sample (including lysate) from the cell or organism with an effector protein disclosed herein, and a guide nucleic acid. The sample may comprise a target nucleic acid encoding a sequence associated with a phenotypic trait. The sample may comprise a target nucleic acid encoding a sequence indicative of a genotype. The target nucleic acid, in some cases, is a nucleic acid encoding a sequence associated with a geographic region of origin or ethnic group.

Any of the above disclosed samples are consistent with the methods, compositions, reagents, enzymes, and systems disclosed herein.

### XI. Methods and Formulations for Introducing Systems and Compositions into a Target Cell

A guide nucleic acid and/or an effector protein described herein can be introduced into a host cell by any of a variety of well-known methods. As a non-limiting example, a guide nucleic acid and/or effector protein can be combined with a lipid. As another non-limiting example, a guide nucleic acid and/or effector protein can be combined with a particle, or formulated into a particle.

### Methods for Introducing Systems and Compositions to a Host

Described herein are methods of introducing various components described herein to a host. A host can be any suitable host, such as a host cell. When described herein, a host cell can be an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for methods of introduction described herein, and include the progeny of the original cell which has been transformed by the methods of introduction described herein. It is understood that the progeny of a single cell may not necessarily be completely identical in morphology or in genomic or total DNA complement as the original parent, due to natural, accidental, or deliberate mutation. A host cell can be a recombinant host cell or a genetically modified host cell, if a heterologous nucleic acid, e.g., an expression vector, has been introduced into the cell.

Methods of introducing a nucleic acid and/or protein into a host cell are known in the art, and any convenient method can be used to introduce a subject nucleic acid (e.g., a nucleic acid expression vector) into a target cell (e.g., a human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: 50169-409X(12)00283-9. doi: 10.1016/j.addr.2012.09.023), and the like. In some embodiments, the nucleic acid and/or protein are introduced into a disease cell comprised in a pharmaceutical composition comprising the guide nucleic acid and/or effector protein and a pharmaceutically acceptable excipient.

In certain embodiments, molecules of interest, such as nucleic acids of interest, are introduced to a host. In certain embodiments, polypeptides, such as an effector protein are introduced to a host. In certain embodiments, vectors, such as lipid particles and/or viral vectors can be introduced to a host. Introduction can be for contact with a host or for assimilation into the host, for example, introduction into a host cell.

In some embodiments, described herein are methods of introducing one or more nucleic acids, such as a nucleic acid encoding an effector protein, a nucleic acid that, when transcribed, produces an engineered guide nucleic acid, and/or a donor nucleic acid, or combinations thereof, into a host cell. Any suitable method can be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout.

Introducing one or more nucleic acids into a host cell can occur in any culture media and under any culture conditions that promote the survival of the cells. Introducing one or more nucleic acids into a host cell can be carried out in vivo or ex vivo. Introducing one or more nucleic acids into a host cell can be carried out in vitro.

In some embodiments, an effector protein can be provided as RNA. The RNA can be provided by direct chemical synthesis or may be transcribed in vitro from a DNA (e.g., encoding the effector protein). Once synthesized, the RNA may be introduced into a cell by way of any suitable technique for introducing nucleic acids into cells (e.g., microinjection, electroporation, transfection, etc.). In some embodiments, introduction of one or more nucleic acid can be through the use of a vector and/or a vector system, accordingly, in some embodiments, compositions and system described herein comprise a vector and/or a vector system.

Vectors may be introduced directly to a host. In certain embodiments, host cells can be contacted with one or more vectors as described herein, and in certain embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

Components described herein can also be introduced directly to a host. For example, an engineered guide nucleic acid can be introduced to a host, specifically introduced into a host cell. Methods of introducing nucleic acids, such as RNA into cells include, but are not limited to direct injection, transfection, or any other method used for the introduction of nucleic acids.

Polypeptides (e.g., effector proteins) described herein can also be introduced directly to a host. In some embodiments, polypeptides described herein can be modified to promote introduction to a host. For example, polypeptides described herein can be modified to increase the solubility of the polypeptide. Such a polypeptide may optionally be fused to a polypeptide domain that increases solubility. The domain may be linked to the polypeptide through a defined protease cleavage site, such as TEV sequence which is cleaved by TEV protease. The linker may also include one or more flexible sequences, e.g. from 1 to 10 glycine residues. In some embodiments, the cleavage of the polypeptide is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. In another example, the polypeptide can be modified to improve stability. For example, the polypeptides may be PEGylated, where the polyethyleneoxy group provides for enhanced lifetime in the blood stream. Polypeptides can also be modified to promote uptake by a host, such as a host cell. For example, a polypeptide described herein can be fused to a polypeptide permeant domain to promote uptake by a host cell. Any suitable permeant domains can be used in the non-integrating polypeptides of the present disclosure, including peptides, peptidomimetics, and non-peptide carriers. Examples include penetratin, a permeant peptide may be derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine, and the like. The site at which the fusion is made may be selected in order to optimize the biological activity, secretion or binding characteristics of the polypeptide. The optimal site can be determined by suitable methods.

### Formulations for Introducing Systems and Compositions to a Host

Described herein are formulations of introducing systems and compositions described herein to a host. In some embodiments, such formulations, systems and compositions described herein comprise an effector protein and a carrier (e.g., excipient, diluent, vehicle, or filling agent). In some aspects of the present invention the effector protein is provided in a pharmaceutical composition comprising the effector protein and any pharmaceutically acceptable excipient, carrier, or diluent.

### XII. Methods of Nucleic Acid Editing

Provided herein are compositions, methods, and systems for editing target nucleic acids. In general, editing refers to modifying the nucleotide sequence of a target nucleic acid. However, compositions, methods, and systems disclosed herein may also be capable of making epigenetic modifications of target nucleic acids. Effector proteins, multimeric complexes thereof and systems described herein may be used for editing or modifying a target nucleic acid. Editing a target nucleic acid may comprise one or more of cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid.

The target nucleic acid may be a gene or a portion thereof. Methods and compositions may modify a coding portion of a gene, a non-coding portion of a gene, or a combination thereof. Modifying at least one gene using the compositions and methods described herein may reduce or increase expression of one or more genes. In some embodiments, compositions and methods reduce expression of one or more genes by at least 10%, at least 20%, at least 30%, at least 40%, at least 50%, at least 60%, at least 70%, at least 80%, at least 90%, or at least 95%. In some embodiments, compositions and methods remove all expression of a gene, also referred to as genetic knock out. In some embodiments, compositions and methods increase expression of one or more genes by at least 10%, at least 20%, at least 30%, at least 40%, at least 50%, at least 60%, at least 70%, at least 80%, at least 90%, or at least 100%.

In some embodiments, compositions and methods use effector proteins that are fused to a heterologous protein. Heterologous proteins include, but are not limited to, transcriptional activators, transcriptional repressors, deaminases, methyltransferases, acetyltransferases, and other nucleic acid modifying proteins. In some embodiments, effector proteins need not be fused to a partner protein to accomplish the required protein (expression) modification.

In some embodiments, compositions and methods comprise a nucleic acid expression vector, or use thereof, to introduce an effector protein, guide nucleic acid, donor template or any combination thereof to a cell. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions and methods comprise a lipid, polymer, nanoparticle, or a combination thereof, or use thereof, to introduce a Cas protein, guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

Methods of editing may comprise contacting a target nucleic acid with an effector protein described herein and a guide nucleic acid, wherein the effector protein comprises an amino acid sequence that is at least 65%, at least 70%, at least 75%, at least 80%, at least 85%, at least 90%, at least 95%, at least 97%, or at least 98%, at least 99%, or 100% identical to the sequence selected from SEQ ID NOS: 1-1614 and 3195-3302.

Methods may comprise removing a mutation (e.g., point mutations, deletions) in a target nucleic acid. Methods may remove or correct a disease-causing mutation in a nucleic acid sequence to produce a corresponding wildtype nucleobase sequence. Methods may correct the sequence of a target nucleic acid comprising a single nucleotide polymorphism (SNP) to a wildtype sequence. Methods may comprise modifying a target nucleic acid comprising a mutation such that expression of the target nucleic acid is reduced or arrested. Methods may generate gene knock-out, gene knock-in, gene editing, gene tagging, or a combination thereof. Methods of the disclosure may be targeted to any locus in a genome of a cell.

Editing may introduce a mutation (e.g., point mutations, deletions) in a target nucleic acid relative to a corresponding wildtype nucleotide sequence. Editing may remove or correct a disease-causing mutation in a nucleic acid sequence to produce a corresponding wildtype nucleotide sequence. Editing may remove/correct point mutations, deletions, null mutations, or tissue-specific mutations in a target nucleic acid. Editing may be used to generate gene knock-out, gene knock-in, gene editing, gene tagging, or a combination thereof. Methods of the disclosure may be targeted to any locus in a genome of a cell.

Editing may comprise single stranded cleavage, double stranded cleavage, donor nucleic acid insertion, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer region. In some embodiments, the effector proteins introduce a single-stranded break in a target nucleic acid to produce a cleaved nucleic acid. In some embodiments, the effector protein is capable of introducing a break in a single stranded RNA (ssRNA). The effector protein may be coupled to a guide nucleic acid that targets a particular region of interest in the ssRNA. In some embodiments, the target nucleic acid, and the resulting cleaved nucleic acid is contacted with a nucleic acid for homologous recombination (e.g., homology directed repair (HDR)) or non-homologous end joining (NHEJ). In some embodiments, a double-stranded break in the target nucleic acid may be repaired (e.g., by NHEJ or HDR) without insertion of a donor template, such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of one or more nucleotide in a target nucleic acid. An indel can vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are modified by a given effector protein.

In some embodiments, wherein the compositions, systems, and methods of the present disclosure comprise an additional guide nucleic acid or a use thereof, the dual-guided compositions, systems, and methods described herein can modify the target nucleic acid in two locations. In some embodiments, dual-guided editing can comprise cleavage of the target nucleic acid in the two locations targeted by the guide nucleic acids. In certain embodiments, upon removal of the sequence between the guide nucleic acids, the wild-type reading frame is restored. A wild-type reading frame can be a reading frame that produces at least a partially, or fully, functional protein. A non-wild-type reading frame can be a reading frame that produces a non-functional or partially non-functional protein.

Accordingly, in some embodiments, compositions, systems, and methods described herein can edit 1 to 1,000 nucleotides or any integer in between, in a target nucleic acid. In certain embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, can be edited by the compositions, systems, and methods described herein. In some embodiments, 1, 2, 3, 4, 5, 6, 7, 8, 9, 10, or more nucleotides can be edited by the compositions, systems, and methods described herein. In some embodiments, 10, 20, 30, 40, 50, 60, 70, 80 90, 100 or more nucleotides, or any integer in between, can be edited by the compositions, systems, and methods described herein. In some embodiments, 100, 200, 300, 400, 500, 600, 700, 800, 900 or more nucleotides, or any integer in between, can be edited by the compositions, systems, and methods described herein.

In some embodiments, the effector protein is fused to a chromatin-modifying enzyme. In some embodiments, the fusion protein chemically modifies the target nucleic acid, for example by methylating, demethylating, or acetylating the target nucleic acid in a sequence specific or non-specific manner.

Methods may comprise use of two or more effector proteins. An illustrative method for introducing a break in a target nucleic acid comprises contacting the target nucleic acid with: (a) a first engineered guide nucleic acid comprising a region that binds to a first effector protein, wherein the effector protein comprises at least 75% sequence identity to any one of the sequences selected from SEQ ID NOS: 1-1614 and 3195-3302; and (b) a second engineered guide nucleic acid comprising a region that binds to a second effector protein, wherein the effector protein comprises at least 75% sequence identity to any one of the sequences selected from SEQ ID NOS: 1-1614 and 3195-3302, wherein the first engineered guide nucleic acid comprises an additional region that binds to the target nucleic acid and wherein the second engineered guide nucleic acid comprises an additional region that binds to the target nucleic acid.

In some embodiments, editing a target nucleic acid comprises genome editing. Genome editing may comprise modifying a genome, chromosome, plasmid, or other genetic material of a cell or organism. In some embodiments, the genome, chromosome, plasmid, or other genetic material of the cell or organism is modified in vivo. In some embodiments, the genome, chromosome, plasmid, or other genetic material of the cell or organism is modified in a cell. In some embodiments, the genome, chromosome, plasmid, or other genetic material of the cell or organism is modified in vitro. For example, a plasmid may be modified in vitro using a composition described herein and introduced into a cell or organism. In some embodiments, modifying a target nucleic acid may comprise deleting a sequence from a target nucleic acid. For example, a mutated sequence or a sequence associated with a disease may be removed from a target nucleic acid. In some embodiments, modifying a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, modifying a target nucleic acid may comprise introducing a sequence into a target nucleic acid. For example, a beneficial sequence or a sequence that may reduce or eliminate a disease may be inserted into the target nucleic acid.

In some embodiments, methods comprise inserting a donor nucleic acid into a cleaved target nucleic acid. The donor nucleic acid may be inserted at a specified (e.g., effector protein targeted) point within the target nucleic acid. In some embodiments, methods comprise contacting a target nucleic acid with an effector protein comprising an amino acid sequence that is at least 65%, at least 70%, at least 75%, at least 80%, at least 85%, at least 90%, at least 95%, at least 97%, at least 98%, at least 99%, or 100% identical to any one of the sequences selected from SEQ ID NOS: 1-1614 and 3195-3302, thereby introducing a single-stranded break in the target nucleic acid; contacting the target nucleic acid with a second effector protein, optionally comprising an amino acid sequence that is at least 65%, at least 70%, at least 75%, at least 80%, at least 85%, at least 90%, at least 95%, at least 97%, at least 98%, or at least 99% identical to any one of the sequences selected from SEQ ID NOS: 1-1614 and 3195-3302, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally via HDR or NHEJ, thereby introducing a new sequence into the target nucleic acid (e.g., at a cleavage site or in between two cleavage sites).

In some embodiments, methods comprise editing a target nucleic acid with two or more effector proteins. Editing a target nucleic acid may comprise introducing a two or more single-stranded breaks in a target nucleic acid. In some embodiments, a break may be introduced by contacting a target nucleic acid with an effector protein and a guide nucleic acid. The guide nucleic acid may bind to the effector protein and hybridize to a region of the target nucleic acid, thereby recruiting the effector protein to the region of the target nucleic acid. Binding of the effector protein to the guide nucleic acid and the region of the target nucleic acid may activate the effector protein, and the effector protein may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first effector protein and hybridizes to a first region of the target nucleic acid and a second guide nucleic acid that binds to a second programmable nickase and hybridizes to a second region of the target nucleic acid. The first effector protein may introduce a first break in a first strand at the first region of the target nucleic acid, and the second effector protein may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with donor nucleic acid), thereby modifying the target nucleic acid. In some embodiments, the effector protein comprises an amino acid sequence that is at least 65%, at least 70%, at least 75%, at least 80%, at least 85%, at least 90%, at least 95%, at least 97%, at least 98%, at least 99%, or 100% identical to any one of the sequences selected from SEQ ID NOS: 1-1614 and 3195-3302.

In some embodiments, editing is achieved by fusing an effector protein to a heterologous sequence. The heterologous sequence may be a suitable fusion partner, e.g., a protein that provides recombinase activity by acting on the target nucleic acid. In some embodiments, the fusion protein comprises an effector protein fused to a heterologous sequence by a linker. The heterologous sequence or fusion partner may be a base editing domain. The base editing domain may be an ADAR1/2 or any functional variant thereof. The heterologous sequence or fusion partner may be fused to the C-terminus, N-terminus, or an internal portion (e.g., a portion other than the N- or C-terminus) of the effector protein. The heterologous sequence or fusion partner may be fused to the effector protein by a linker. A linker may be a peptide linker or a non-peptide linker. In some embodiments, the linker is an XTEN linker. In some embodiments, the linker comprises one or more repeats a tri-peptide GGS. In some embodiments, the linker is from 1 to 100 amino acids in length. In some embodiments, the linker is more 100 amino acids in length. In some embodiments, the linker is from 10 to 27 amino acids in length. A non-peptide linker may be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

Methods, systems and compositions described herein can edit or modify a target nucleic acid wherein such editing or modification can effect one or more indels. In some embodiments, where compositions, systems, and/or methods described herein effect one or more indels, then in certain embodiments, the impact on the transcription and/or translation of the target nucleic acid can be predicted depending on: 1) the amount of indels generated; and 2) the location of the indel on the target nucleic acid. For example, as described herein, in certain embodiments, if the amount of indels is not divisible by three, and the indels occur within or along a protein coding region, then the modification or mutation can be a frameshift mutation.

In certain embodiments, if the amount of indels is divisible by three, then a frameshift mutation may not be effected, but a splicing disruption mutation and/or sequence skip mutation may be effected, such as an exon skip mutation. In certain embodiments, if the amount of indels is not evenly divisible by three, then a frameshift mutation may be effected.

Methods, systems and compositions described herein can edit or modify a target nucleic acid wherein such editing or modification can be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity can be detected by next generation sequencing of one or more target loci of a target nucleic acid where indel percentage is calculated as the fraction of sequencing reads containing insertions or deletions relative to an unedited reference sequence. In certain embodiments, methods, systems, and compositions comprising an effector protein and guide nucleic acid described herein can exhibit about 0.0001% to about 65% or more indel activity upon contact to a target nucleic acid compared to a target nucleic acid non-contacted with compositions, systems, or by methods described herein. For example, methods, systems, and compositions comprising an effector protein and guide nucleic acid described herein can exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In some embodiments, editing or modifications of a target nucleic acid as described herein effects one or more mutations comprising splicing disruption mutations, frameshift mutations (e.g., 1+ or 2+ frameshift mutation), sequence deletion, sequence skipping, sequence reframing, sequence knock-in, or any combination thereof.

A splicing disruption can be a modification that disrupts the splicing of a target nucleic acid or splicing of a sequence that is transcribed from a target nucleic acid relative to a target nucleic acid without the splicing disruption.

A frameshift mutation can be a modification that alters the reading frame of a target nucleic acid relative to a target nucleic acid without the frameshift mutation. In certain embodiments, a frameshift mutation can be a +2 frameshift mutation wherein a reading frame is modified by 2 bases. In certain embodiments, a frameshift mutation can be a +1 frameshift mutation wherein a reading frame is modified by 1 base. In certain embodiments, a frameshift mutation is a modification that alters the number of bases in a target nucleic acid so that it is not divisible by three. In some embodiments, a frameshift mutation can be a modification that is not a splicing disruption.

A sequence as described in reference to a sequence deletion, sequence skipping, sequence reframing, and sequence knock-in can be a DNA sequence, a RNA sequence, a modified DNA or RNA sequence, a mutated sequence, a wild-type sequence, a coding sequence, a non-coding sequence, an exonic sequence (exon), an intronic sequence (intron), or any combination thereof. Such a sequence can be a sequence that is associated with a disease as described herein, such as DMD.

In certain embodiments, sequence deletion is a modification where one or more sequences in a target nucleic acid are deleted relative to a target nucleic acid without the sequence deletion. In certain embodiments, a sequence deletion can result in or effect a splicing disruption or a frameshift mutation. In certain embodiments, a sequence deletion result in or effect a splicing disruption.

In certain embodiments, sequence skipping is a modification where one or more sequences in a target nucleic acid are skipped upon transcription or translation of the target nucleic acid relative to a target nucleic acid without the sequence skipping. In certain embodiments, sequence skipping can result in or effect a splicing disruption or a frameshift mutation. In certain embodiments, sequence skipping can result in or effect a splicing disruption.

In certain embodiments, sequence reframing is a modification where one or more bases in a target are modified so that the reading frame of the sequence is reframed relative to a target nucleic acid without the sequence reframing. In certain embodiments, sequence reframing can result in or effect a splicing disruption or a frameshift mutation. In certain embodiments, sequence reframing can result in or effect a frameshift mutation.

In certain embodiments, sequence knock-in is a modification where one or more sequences is inserted into a target nucleic acid relative to a target nucleic acid without the sequence knock-in. In certain embodiments, sequence knock-in can result in or effect a splicing disruption or a frameshift mutation. In certain embodiments, sequence knock-in can result in or effect a splicing disruption.

In certain embodiments, editing or modification of a target nucleic acid can be locus specific, wherein compositions, systems, and methods described herein can edit or modify a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing or modification of a specific locus can effect any one of a splicing disruption, frameshift (e.g., 1+ or 2+ frameshift), sequence deletion, sequence skipping, sequence reframing, sequence knock-in, or any combination thereof. In certain embodiments, editing or modification of a target nucleic acid can be locus specific, modification specific, or both. In certain embodiments, editing or modification of a target nucleic acid can be locus specific, modification specific, or both, wherein compositions, systems, and methods described herein comprise an effector protein described herein and a guide nucleic acid described herein.

Methods of editing a target nucleic acid or modulating the expression of a target nucleic acid may be performed in vivo. Methods of editing a target nucleic acid or modulating the expression of a target nucleic acid may be performed in vitro. For example, a plasmid may be modified in vitro using a composition described herein and introduced into a cell or organism. Methods of editing a target nucleic acid or modulating the expression of a target nucleic acid may be performed ex vivo. For example, methods may comprise obtaining a cell from a subject, modifying a target nucleic acid in the cell with methods described herein, and returning the cell to the subject.

In some embodiments, the CRISPR-associated transposase (CAST) systems disclosed herein may be used for targeted nucleic acid modification. In some embodiments, the methods of modifying nucleic acids comprise modifying a target nucleic acid in vitro, comprising: bringing the CRISPR-associated transposase (CAST) systems disclosed herein in contact with the target nucleic acid, wherein the donor nucleic acid sequence is integrated into the target nucleic acid.

In some embodiments, the methods comprise introducing into a cell, any one of the CRISPR-associated transposase (CAST) systems disclosed herein. The disclosure further provides methods of modifying a target nucleic acid in a cell comprising introducing into the cell, any one the CRISPR-associated transposase (CAST) systems disclosed herein, wherein the donor nucleic acid sequence is integrated into the target nucleic acid. For instance, in some embodiments, the donor nucleic acid is inserted into the target nucleic acid, or used to edit the target nucleic acid. In some cases, the donor nucleic acid comprises one or more mutations (e.g. nucleotide substitutions or insertions) to be introduced into the target nucleic acid. The mutations may cause a shift in an open reading frame on the target nucleic acid. In some cases, the donor nucleic acid alters a stop codon in the target nucleic acid. For example, the donor nucleic acid may correct a premature stop codon. The correction may be achieved by deleting the stop codon or introducing one or more mutations to the stop codon.

In some embodiments, the methods comprise using any one the CRISPR-associated transposase (CAST) systems disclosed herein to insert or restore a functional copy of a gene, or a fragment thereof, or a regulatory sequence or a fragment thereof to correct loss of function mutations, deletions, or translocations associated with disease states or disorders. In some embodiments, the integration of the donor nucleic acid: (i) introduces one or more mutations to the target nucleic acid, (ii) corrects or introduces a premature stop codon in the target nucleic acid, (iii) disrupts a splicing site, (iv) restores or introduces a splicing site, (v) inserts a gene or gene fragment at one or both alleles of a target nucleic acid, or (vi) any combination thereof. The one or more mutations introduced by the donor nucleic acid may be one or more substitutions, deletions, insertions, or a combination thereof. In some embodiments, the one or more mutations cause a shift in an open reading frame on the target nucleic acid.

### Donor Nucleic Acids

In some embodiments, a donor nucleic acid comprises a nucleic acid that is incorporated into a target nucleic acid or target sequence. In reference to a viral vector, the term donor nucleic acid refers to a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome. As another example, when used in reference to the activity of an effector protein, the term donor nucleic acid refers to a sequence of nucleotides that will be or has been inserted at the site of cleavage by the effector protein (cleaving (hydrolysis of a phosphodiester bond) of a nucleic acid resulting in a nick or double strand break-nuclease activity). As yet another example, when used in reference to homologous recombination, the term donor nucleic acid refers to a sequence of DNA that serves as a template in the process of homologous recombination, which may carry the modification that is to be or has been introduced into the target nucleic acid. By using this donor nucleic acid as a template, the genetic information, including the modification, is copied into the target nucleic acid by way of homologous recombination.

Donor nucleic acids of any suitable size may be integrated into a target nucleic acid or genome. In some embodiments, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some embodiments, donor nucleic acids are more than 500 kilobases (kb) in length.

The donor nucleic acid may comprise a sequence that is derived from a plant, bacteria, virus or an animal. The animal may be human. The animal may be a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). The non-human animal may be a domesticated mammal or an agricultural mammal.

### Genetically Modified Cells and Organisms

Methods of editing described herein may be employed to generate a genetically modified cell. The cell may be a eukaryotic cell (e.g., a mammalian cell) or a prokaryotic cell (e.g., an archaeal cell). The cell may be derived from a multicellular organism and cultured as a unicellular entity. The cell may comprise a heritable genetic modification, such that progeny cells derived therefrom comprise the heritable genetic mutation. The cell may be progeny of a genetically modified cell comprising a genetic modification of the genetically modified parent cell. A genetically modified cell may comprise a deletion, insertion, mutation, or non-native sequence relative to a wild-type version of the cell or the organism from which the cell was derived.

Methods may comprise contacting a cell with a nucleic acid (e.g., a plasmid or mRNA) comprising a nucleotide sequence encoding an effector protein, wherein the effector protein comprises an amino acid sequence that is at least 65%, at least 70%, at least 75%, at least 80%, at least 85%, at least 90%, at least 95%, at least 97%, at least 98%, or at least 99% identical to any one of the sequences selected from SEQ ID NOs: 1-1614 and 3195-3302.

Methods may comprise contacting cells with a nucleic acid (e.g., a plasmid or RNA) comprising a nucleotide sequence that is a guide nucleic acid (e.g., sgRNA or crRNA), a tracrRNA or any combination thereof or a nucleotide sequence, when transcribed, produces a guide nucleic acid (e.g., sgRNA or crRNA), a tracrRNA or any combination thereof. In some embodiments, the nucleotide sequence of the nucleic acid is at least 65%, at least 70%, at least 75%, at least 80%, at least 85%, at least 90%, at least 92%, at least 95%, at least 97%, at least 98%, at least 99%, or 100% identical to any one of the sequences set forth in Columns B1, B2, and B3 of TABLE 1 and Columns D1 and D2 of TABLE 2, or any combination thereof. Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof.

Methods may comprise contacting a cell with an effector protein or a multimeric complex thereof, wherein the effector protein comprises an amino acid sequence that is at least 65%, at least 70%, at least 75%, at least 80%, at least 85%, at least 90%, at least 95%, at least 97%, at least 98%, or at least 99% identical to any one of the sequences selected from SEQ ID NOs: 1-1614 and 3195-3302.

Methods may comprise contacting a cell with an effector protein, or a nucleic acid (e.g., a plasmid or mRNA) encoding the effector protein, and a nucleic acid (e.g., a plasmid or RNA) comprising a nucleotide sequence that is a guide nucleic acid (e.g., sgRNA or crRNA), a tracrRNA or any combination thereof or a nucleotide sequence, when transcribed, produces a guide nucleic acid (e.g., sgRNA or crRNA), a tracrRNA or any combination thereof, wherein the effector protein comprises an amino acid sequence that is at least 65%, at least 70%, at least 75%, at least 80%, at least 85%, at least 90%, at least 95%, at least 97%, at least 98%, or at least 99% identical to any one of the sequences selected from SEQ ID NOs: 1-1614 and 3195-330, and the nucleotide sequence of the nucleic acid is at least 65%, at least 70%, at least 75%, at least 80%, at least 85%, at least 90%, at least 92%, at least 95%, at least 97%, at least 98%, at least 99%, or 100% identical to any one of the sequences set forth in Columns B1, B2, and B3 of TABLE 1 and Columns D1 and D2 of TABLE 2, or any combination thereof. Such methods include contacting a cell with an RNP complex as described herein, including RNP complexes comprising an effector protein and a nucleic acid (e.g., RNA) comprising a nucleotide sequence that is a guide nucleic acid (e.g., sgRNA or crRNA), a tracrRNA or any combination thereof. In some embodiments, for methods that include use of such an RNP complex, the effector protein comprises an amino acid sequence that is at least 65%, at least 70%, at least 75%, at least 80%, at least 85%, at least 90%, at least 95%, at least 97%, at least 98%, or at least 99% identical to any one of the sequences selected from SEQ ID NOs: 1-1614 and 3195-3302, and the nucleotide sequence of the nucleic acid is at least 65%, at least 70%, at least 75%, at least 80%, at least 85%, at least 90%, at least 92%, at least 95%, at least 97%, at least 98%, at least 99%, or 100% identical to any one of the sequences set forth in Columns B1, B2, and B3 of TABLE 1 and Columns D1 and D2 of TABLE 2, or any combination thereof

Methods of the disclosure may be performed in a subject. Compositions of the disclosure may be administered to a subject. A subject may be a human. A subject may be a mammal (e.g., rat, mouse, cow, dog, pig, sheep, horse). A subject may be a vertebrate or an invertebrate. A subject may be a laboratory animal. A subject may be a patient. A subject may be at risk of developing, suffering from, or displaying symptoms a disease or disorder as set forth in herein. The subject may have a mutation associated with a gene described herein. The subject may display symptoms associated with a mutation of a gene described herein. In some embodiments, a mutation comprises a point mutation or single nucleotide polymorphism (SNP), a chromosomal mutation, a copy number mutation, or any combination thereof. A point mutation optionally comprises a substitution, insertion, or deletion. In some embodiments, a mutation comprises a chromosomal mutation. A chromosomal mutation can comprise an inversion, a deletion, a duplication, or a translocation. In some embodiments, a mutation comprises a copy number variation. A copy number variation can comprise a gene amplification or an expanding trinucleotide repeat. In some embodiments, mutations may be as described herein.

Methods of the disclosure may be performed in a cell. A cell may be in vitro. A cell may be in vivo. A cell may be ex vivo. A cell may be an isolated cell. A cell may be a cell inside of an organism. A cell may be an organism. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. A cell may be a eukaryotic cell or derived from a eukaryotic cell. A cell may be a pluripotent stem cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell.

A cell may be from a specific organ or tissue. The tissue may be muscle. The muscle may be skeletal muscle. In certain embodiments, skeletal muscles include the following: abductor digiti minimi (foot), abductor digiti minimi (hand), abductor hallucis, abductor pollicis brevis, abductor pollicis longus, adductor brevis, adductor hallucis, adductor longus, adductor magnus, adductor pollicis, anconeus, articularis cubiti, articularis genu, aryepiglotticus, auricularis, biceps brachii, biceps femoris, brachialis, brachioradialis, buccinator, bulbospongiosus, constrictor of pharynx—inferior, constrictor of pharynx—middle, constrictor of pharynx—superior, coracobrachialis, corrugator supercilii, cremaster, cricothyroid, dartos, deep transverse perinei, deltoid, depressor anguli oris, depressor labii inferioris, diaphragm, digastric, digastric (anterior view), erector spinae—spinalis, erector spinae—iliocostalis, erector spinae—longissimus, extensor carpi radialis brevis, extensor carpi radialis longus, extensor carpi ulnaris, extensor digiti minimi (hand), extensor digitorum (hand), extensor digitorum brevis (foot), extensor digitorum longus (foot), extensor hallucis brevis, extensor hallucis longus, extensor indicis, extensor pollicis brevis, extensor pollicis longus, external oblique abdominis, flexor carpi radialis, flexor carpi ulnaris, flexor digiti minimi brevis (foot), flexor digiti minimi brevis (hand), flexor digitorum brevis, flexor digitorum longus (foot), flexor digitorum profundus, flexor digitorum superficialis, flexor hallucis brevis, flexor hallucis longus, flexor pollicis brevis, flexor pollicis longus, frontalis, gastrocnemius, gemellus inferior, gemellus superior, genioglossus, geniohyoid, gluteus maximus, gluteus medius, gluteus minimus, gracilis, hyoglossus, iliacus, inferior oblique, inferior rectus, infraspinatus, intercostals external, intercostals innermost, intercostals internal, internal oblique abdominis, interossei—dorsal of hand, interossei—dorsal of foot, interossei—palmar of hand, interossei—plantar of foot, interspinales, intertransversarii, intrinsic muscles of tongue, ishiocavernosus, lateral cricoarytenoid, lateral pterygoid, lateral rectus, latissimus dorsi, levator anguli oris, levator ani-coccygeus, levator ani—iliococcygeus, levator ani-pubococcygeus, levator ani-puborectalis, levator ani-pubovaginalis, levator labii superioris, levator labii superioris, alaeque nasi, levator palpebrae superioris, levator scapulae, levator veli palatini, levatores costarum, longus capitis, longus colli, lumbricals of foot, lumbricals of hand, masseter, medial pterygoid, medial rectus, mentalis, m. uvulae, mylohyoid, nasalis, oblique arytenoid, obliquus capitis inferior, obliquus capitis superior, obturator externus, obturator internus (A), obturator internus (B), omohyoid, opponens digiti minimi (hand), opponens pollicis, orbicularis oculi, orbicularis oris, palatoglossus, palatopharyngeus, palmaris brevis, palmaris longus, pectineus, pectoralis major, pectoralis minor, peroneus brevis, peroneus longus, peroneus tertius, piriformis (A), piriformis (B), plantaris, platysma, popliteus, posterior cricoarytenoid, procerus, pronator quadratus, pronator teres, psoas major, psoas minor, pyramidalis, quadratus femoris, quadratus lumborum, quadratus plantae, rectus abdominis, rectus capitus anterior, rectus capitus lateralis, rectus capitus posterior major, rectus capitus posterior minor, rectus femoris, rhomboid major, rhomboid minor, risorius, salpingopharyngeus, sartorius, scalenus anterior, scalenus medius, scalenus minimus, scalenus posterior, semimembranosus, semitendinosus, serratus anterior, serratus posterior inferior, serratus posterior superior, soleus, sphincter ani, sphincter urethrae, splenius capitis, splenius cervicis, stapedius, sternocleidomastoid, sternohyoid, sternothyroid, styloglossus, stylohyoid, stylohyoid (anterior view), stylopharyngeus, subclavius, subcostalis, subscapularis, superficial transverse perinei, superior oblique, superior rectus, supinator, supraspinatus, temporalis, temporoparietalis, tensor fasciae lata, tensor tympani, tensor veli palatini, teres major, teres minor, thyro-arytenoid & vocalis, thyro-epiglotticus, thyrohyoid, tibialis anterior, tibialis posterior, transverse arytenoid, transversospinalis—multifidus, transversospinalis—rotatores, transversospinalis—semispinalis, transversus abdominis, transversus thoracis, trapezius, triceps, vastus intermedius, vastus lateralis, vastus medialis, zygomaticus major, or zygomaticus minor. In some embodiments, the cell is a myocyte. In some embodiments, the cell is a muscle cell. In some embodiments, the muscle cell is a skeletal muscle cell. In some embodiments, the skeletal muscle cell is a red (slow) skeletal muscle cell, a white (fast) skeletal muscle cell or an intermediate skeletal muscle cell.

The tissue may be the subject's blood, bone marrow, or cord blood. The tissue may be heterologous donor blood, cord blood, or bone marrow. The tissue may be allogenic blood, cord blood, or bone marrow. In some embodiments, the cell is a: a stem cell, muscle satellite cell, muscle stem cell, myoblast, muscle progenitor cell, a pluripotent stem cell or a cell derived from a pluripotent stem cell.

### XIII. Methods of Detecting a Target Nucleic Acid

Provided herein are methods of detecting target nucleic acids. Methods may comprise detecting target nucleic acids with compositions or systems described herein. Methods may comprise detecting a target nucleic acid in a sample, e.g., a cell lysate, a biological fluid, or environmental sample. Methods may comprise detecting a target nucleic acid in a cell. In some instances, methods of detecting a target nucleic acid in a sample or cell comprises contacting the sample or cell with an effector protein or a multimeric complex thereof, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some instances, methods result in trans cleavage of the reporter nucleic acid. In some instances, methods result in cis cleavage of the reporter nucleic acid.

In some instances, the effector protein comprises an amino acid sequence that is at least is at least 70%, at least 75%, at least 80%, at least 85%, at least 90%, at least 92%, at least 95%, at least 97%, at least 98%, at least 99%, or 100% identical to any one of SEQ ID NOs: 1-1614 and 3195-3302. In some instances, the amino acid sequence of the effector protein is at least 70%, at least 75%, at least 80%, at least 85%, at least 90%, at least 92%, at least 95%, at least 97%, at least 98%, at least 99%, or 100% identical to any one of SEQ ID NOs: 1-1614 and 3195-3302. In some instances, the nucleobase sequence of the guide is at least 70%, at least 75%, at least 80%, at least 85%, at least 90%, at least 92%, at least 95%, at least 97%, at least 98%, at least 99%, or 100% identical to any one of SEQ ID NOs: 1615-3194 and 3303-3406.

Methods may comprise contacting the sample to a complex comprising a guide nucleic acid comprising a segment that is reverse complementary to a segment of the target nucleic acid and a effector protein that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

Methods may comprise contacting the sample comprising the target nucleic acid with a guide nucleic acid targeting a target nucleic acid segment, an effector protein capable of being activated when complexed with the guide nucleic acid and the target nucleic acid segment, a single stranded nucleic acid of a reporter comprising a detection moiety, wherein the nucleic acid of a reporter is capable of being cleaved by the activated effector protein, thereby generating a first detectable signal, cleaving the single stranded nucleic acid of a reporter using the effector protein that cleaves as measured by a change in color, and measuring the first detectable signal on the support medium.

Methods may comprise contacting the sample or cell with an effector protein or a multimeric complex thereof and a guide nucleic acid at a temperature of at least about 25° C., at least about 30° C., at least about 35° C., at least about 40° C., at least about 50° C., or at least about 65° C. In some instances, the temperature is not greater than 80° C. In some instances, the temperature is about 25° C., about 30° C., about 35° C., about 40° C., about 45° C., about 50° C., about 55° C., about 60° C., about 65° C., or about 70° C. In some instances, the temperature is about 25° C. to about 45° C., about 35° C. to about 55° C., or about 55° C. to about 65° C.

Methods may comprise cleaving a strand of a single-stranded target nucleic acid with an effector protein or a multimeric complex thereof, as assessed with an in vitro cis-cleavage assay. A cleavage assay can comprise an assay designed to visualize, quantitate or identify cleavage of a nucleic acid. In some embodiments, the cleavage activity may be cis-cleavage activity. In some embodiments, the cleavage activity may be trans-cleavage activity. An example of such an assay (an in vitro cis-cleavage assay). An example of such an assay may follow a procedure comprising: (i) providing equimolar amounts of an effector protein comprising at least 70% sequence identity to any one of the sequences selected from SEQ ID NOs: 1-1614 and 3195-3302 and a guide nucleic acid comprising at least 70% sequence identity to any one of the sequences selected from SEQ ID NOs: 1615-3194 and 3303-3406, under conditions to form a ribonucleoprotein complex; (ii) adding linear dsDNA comprising a nucleic acid sequence targeted by the guide nucleic acid and adjacent to a PAM (iii) incubating the mixture under conditions to enable cleavage of the plasmid; (iv) quenching the reaction with EDTA and a protease; and (v) analyzing the reaction products (e.g., viewing the cleaved and uncleaved linear dsDNA with gel electrophoresis).

In some cases, there is a threshold of detection for methods of detecting target nucleic acids. In some instances, methods are not capable of detecting target nucleic acids that are present in a sample or solution at a concentration less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases, the threshold of detection is in a range of from 2 aM to 100 pM, from 20 aM to 50 pM, from 50 aM to 20 pM, from 200 aM to 5 pM, or from 500 aM to 2 pM. In some cases, the minimum concentration at which a target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a target nucleic acid is detected in a sample is in a range of from 2 aM to 100 pM, from 20 aM to 50 pM, from 50 aM to 20 pM, from 200 aM to 5 pM, or from 500 aM to 2 pM. In some cases, the minimum concentration at which a single stranded target nucleic acid can be detected in a sample is in a range of from 1 aM to 100 pM. In some cases, the minimum concentration at which a target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some instances, the target nucleic acid is present in a cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some instances, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some instances, the target nucleic acid is present in the cleavage reaction at a concentration of from 20 nM to 50 μM, from 50 nM to 20 μM, or from 200 nM to 5 μM.

In some cases, methods detect a target nucleic acid in less than 60 minutes. In some cases, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

In some cases, methods require at least about 120 minutes, at least about 110 minutes, at least about 100 minutes, at least about 90 minutes, at least about 80 minutes, at least about 70 minutes, at least about 60 minutes, at least about 55 minutes, at least about 50 minutes, at least about 45 minutes, at least about 40 minutes, at least about 35 minutes, at least about 30 minutes, at least about 25 minutes, at least about 20 minutes, at least about 15 minutes, at least about 10 minutes, or at least about 5 minutes to detect a target nucleic acid. In some cases, the sample is contacted with the reagents for from 5 minutes to 120 minutes, from 5 minutes to 100 minutes, from 10 minutes to 90 minutes, from 15 minutes to 45 minutes, or from 20 minutes to 35 minutes.

In some cases, methods of detecting are performed in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some cases, methods of detecting are performed in about 5 minutes to about 10 hours, about 10 minutes to about 8 hours, about 15 minutes to about 6 hours, about 20 minutes to about 5 hours, about 30 minutes to about 2 hours, or about 45 minutes to about 1 hour.

Methods may comprise detecting a detectable signal within 5 minutes of contacting the sample and/or the target nucleic acid with the guide nucleic acid and/or the effector protein. In some cases, detecting occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some instances, detecting occurs within 1 to 120, 5 to 100, 10 to 90, 15 to 80, 20 to 60, or 30 to 45 minutes of contacting the target nucleic acid.

### Amplification of a Target Nucleic Acid

Methods of detecting may comprise amplifying a target nucleic acid for detection using any of the compositions or systems described herein. Amplifying may comprise changing the temperature of the amplification reaction, also known as thermal amplification (e.g., PCR). Amplifying may be performed at essentially one temperature, also known as isothermal amplification. Amplifying may improve at least one of sensitivity, specificity, or accuracy of the detection of the target nucleic acid.

Amplifying may comprise subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some instances, amplification of the target nucleic acid comprises modifying the sequence of the target nucleic acid. For example, amplification may be used to insert a PAM sequence into a target nucleic acid that lacks a PAM sequence. In some cases, amplification may be used to increase the homogeneity of a target nucleic acid in a sample. For example, amplification may be used to remove a nucleic acid variation that is not of interest in the target nucleic acid sequence.

Amplifying may take 1, 2, 3, 4, 5, 6, 7, 8, 9, 10, 11, 12, 13, 14, 15, 16, 17, 18, 19, 20, 25, 30, 40, 50, or 60 minutes. Amplifying may be performed at a temperature of around 20-45° C. Amplifying may be performed at a temperature of less than about 20° C., less than about 25° C., less than about 30° C., 35° C., less than about 37° C., less than about 40° C., or less than about 45° C. The nucleic acid amplification reaction may be performed at a temperature of at least about 20° C., at least about 25° C., at least about 30° C., at least about 35° C., at least about 37° C., at least about 40° C., or at least about 45° C.

### XIV. Pharmaceutical Compositions and Modes of Administration

Disclosed herein, in some aspects, are pharmaceutical compositions for modifying a target nucleic acid in a cell or a subject, comprising any one of the effector proteins, engineered effector proteins, fusion effector proteins, or guide nucleic acids as described herein and any combination thereof. Also disclosed herein, in some aspects, are pharmaceutical compositions comprising a nucleic acid encoding any one of the effector proteins, engineered effector proteins, fusion effector proteins, or guide nucleic acids as described herein and any combination thereof. In some embodiments, pharmaceutical compositions comprise a plurality of guide nucleic acids. Pharmaceutical compositions may be used to modify a target nucleic acid or the expression thereof in a cell in vitro, in vivo or ex vivo.

In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein. The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein.

In some embodiments, pharmaceutical compositions comprise a virus comprising a viral vector encoding a fusion effector protein, an effector protein, a fusion partner, a guide nucleic acid, or a combination thereof; and a pharmaceutically acceptable carrier or diluent. The virus may be a lentivirus. The virus may be an adenovirus. The virus may be a non-replicating virus. The virus may be an adeno-associated virus (AAV). The viral vector may be a retroviral vector. Retroviral vectors may include gamma-retroviral vectors such as vectors derived from the Moloney Murine Leukemia Virus (MoMLV, MMLV, MuLV, or MLV) or the Murine Stem cell Virus (MSCV) genome. Retroviral vectors may include lentiviral vectors such as those derived from the human immunodeficiency virus (HIV) genome. In some embodiments, the viral vector is a chimeric viral vector, comprising viral portions from two or more viruses. In some embodiments, the viral vector is a recombinant viral vector.

In some embodiments, the viral vector is an AAV. The AAV may be any AAV known in the art. In some embodiments, the viral vector corresponds to a virus of a specific serotype. In some examples, the serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, and an AAV12 serotype. In some embodiments the AAV vector is a recombinant vector, a hybrid AAV vector, a chimeric AAV vector, a self-complementary AAV (scAAV) vector, a single-stranded AAV or any combination thereof. scAAV genomes are generally known in the art and contain both DNA strands which can anneal together to form double-stranded DNA.

In some embodiments, methods of producing delivery vectors herein comprise packaging a nucleic acid encoding an effector protein and a nucleic acid that, when transcribed, produces a guide nucleic acid, or a combination thereof, into an AAV vector. In some embodiments, methods of producing the delivery vector comprises, (a) contacting a cell with at least one nucleic acid that, when transcribed, produces a guide nucleic acid; at least one nucleic acid that encodes: (i) a Replication (Rep) gene; and (ii) a Capsid (Cap) gene that encodes an AAV capsid protein; (b) expressing the AAV capsid protein in the cell; (c) assembling an AAV particle; and (d) packaging a Cas effector encoding nucleic acid into the AAV particle, thereby generating an AAV delivery vector. In some embodiments, promoters, stuffer sequences, and any combination thereof may be packaged in the AAV vector. In some embodiments, the AAV vector comprises a sequence encoding a guide nucleic acid. In some embodiments, the guide nucleic acid comprises a crRNA. In some embodiments, the guide nucleic acid is a crRNA. In some embodiments, the guide nucleic acid comprises a sgRNA. In some embodiments, the guide nucleic acid is a sgRNA. In some examples, the AAV vector can package 1, 2, 3, 4, or 5 nucleotide sequences encoding guide nucleic acids or copies thereof. In some examples, the AAV vector packages 1 or 2 nucleotide sequences encoding guide nucleic acids or copies thereof. In some embodiments, the AAV vector packages a nucleotide sequence encoding a first guide nucleic acid and a nucleotide sequence encoding a second guide nucleic acid, wherein the first guide nucleic acid and the second guide nucleic acid are the same. In some embodiments, the AAV vector packages a nucleotide sequence encoding a first guide nucleic acid and a nucleotide sequence encoding a second guide nucleic acid, wherein the first guide nucleic acid and the second guide nucleic acid are different. In some embodiments, the AAV vector comprises inverted terminal repeats, e.g., a 5′ inverted terminal repeat and a 3′ inverted terminal repeat. In some embodiments, the inverted terminal repeat comprises inverted terminal repeats from AAV. In some embodiments, the inverted terminal repeat comprises inverted terminal repeats of ssAAV vector or scAAV vector. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some embodiments, the AAV vector may be a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the delivery vector may be a eukaryotic vector, a prokaryotic vector (e.g., a bacterial vector) a viral vector, or any combination thereof. In some embodiments, the delivery vehicle may be a non-viral vector. In some embodiments, the delivery vehicle may be a plasmid. In some embodiments, the plasmid comprises DNA. In some embodiments, the plasmid comprises RNA. In some examples, the plasmid comprises circular double-stranded DNA. In some examples, the plasmid may be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid may be a minicircle plasmid. In some examples, the plasmid contains one or more genes that provide a selective marker to induce a target cell to retain the plasmid. In some examples, the plasmid may be formulated for delivery through injection by a needle carrying syringe. In some examples, the plasmid may be formulated for delivery via electroporation. In some examples, the plasmids may be engineered through synthetic or other suitable means known in the art. For example, in some embodiments, the genetic elements may be assembled by restriction digest of the desired genetic sequence from a donor plasmid or organism to produce ends of the DNA which may then be readily ligated to another genetic sequence.

In some embodiments, the vector is a non-viral vector, and a physical method or a chemical method is employed for delivery into the somatic cell. Exemplary physical methods include electroporation, gene gun, sonoporation, magnetofection, or hydrodynamic delivery. Exemplary chemical methods include delivery of the recombinant polynucleotide via liposomes such as, cationic lipids or neutral lipids; dendrimers; nanoparticles; or cell-penetrating peptides.

In some embodiments, a fusion effector protein as described herein is inserted into a vector. In some embodiments, the vector comprises a nucleotide sequence of one or more promoters, enhancers, ribosome binding sites, RNA splice sites, polyadenylation sites, a replication origin, and/or transcriptional terminator sequences.

In some embodiments, the AAV vector comprises a self-processing array system for guide nucleic acid. Such a self-processing array system refers to a system for multiplexing, stringing together multiple guide nucleic acids under the control of a single promoter. In general, plasmids and vectors described herein comprise at least one promoter. In some embodiments, the promoters are constitutive promoters. In other embodiments, the promoters are inducible promoters. In additional embodiments, the promoters are prokaryotic promoters (e.g., drive expression of a gene in a prokaryotic cell). In some embodiments, the promoters are eukaryotic promoters, (e.g., drive expression of a gene in a eukaryotic cell). Exemplary promoters include, but are not limited to, CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, U6, CaMV35S, SV40, CMV, 7SK, and HSV TK promoter. In some embodiments, the promoter is CMV. In some embodiments, the promoter is EF1a. In some embodiments, the promoter is U6. In some embodiments, the promote is H1. In some embodiments, the promoter is 7SK. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, the AAV vector comprises a promoter for expressing effector proteins. In some embodiments, the promoter for expressing effector protein is a site-specific promoter. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, the AAV vector comprises a stuffer sequence. A stuffer sequence can refer to a non-coding sequence of nucleotides that adjusts the length of the viral genome when inserted into a vector to increase packaging efficiency, increase overall viral titer during production, increase transfection efficacy, increase transfection efficiency, and/or decrease vector toxicity. In some embodiments, the stuffer sequence comprises 5′ untranslated region, 3′ untranslated region or combination thereof. In some embodiments, a stuffer sequence serves no other functional purpose than to increase the length of the viral genome. In some embodiments, a stuffer sequence may increase the length of the viral genome as well as have other functional elements.

In some embodiments, the 3′-untranslated region comprises a nucleotide sequence of an intron. In some embodiments, the 3′-untranslated region comprises one or more sequence elements, such as an intron sequence or an enhancer sequence. In some embodiments, the 3′-untranslated region comprises an enhancer. In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I (Mol. Cell. Biol., Vol. 8(1), p. 466-472, 1988); SV40 enhancer; the intron sequence between exons 2 and 3 of rabbit β-globin (Proc. Natl. Acad. Sci. USA., Vol. 78(3), p. 1527-31, 1981); and the genome region of human growth hormone (J Immunol., Vol. 155(3), p. 1286-95, 1995). In some embodiments, the enhancer is WPRE.

In some embodiments, the AAV vector comprises one or more polyadenylation (poly A) signal sequences. In some embodiments, the polyadenlyation signal sequence comprises hGH poly A signal sequence. In some embodiments, the polyadenlyation signal sequence comprises sv40 poly A signal sequence.

Pharmaceutical compositions described herein may comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²⁺SO₄²⁻.

Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives.

In some embodiments, pharmaceutical compositions are in the form of a solution (e.g., a liquid). The solution may be formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH of the solution is less than 7. In some embodiments, the pH is greater than 7.

In some embodiments, pharmaceutical compositions comprise an: effector protein, fusion effector protein, fusion partner, a guide nucleic acid, or a combination thereof; and a pharmaceutically acceptable carrier or diluent. In some embodiments, pharmaceutical compositions comprise one or more nucleic acids encoding an: effector protein, fusion effector protein, fusion partner, a guide nucleic acid, or a combination thereof; and a pharmaceutically acceptable carrier or diluent. In some embodiments, guide nucleic acid can be a plurality of guide nucleic acids. In some embodiments, the effector protein comprises a sequence that is at least 65%, at least 70%, at least 75%, at least 80%, at least 85%, at least 90%, at least 95%, at least 97%, or at least 98%, at least 99%, or 100% identical to any one of the sequences selected from SEQ ID NOS: 1-1614 and 3195-3302.

### XV. Method of Treating a Disorder

Described herein are compositions and methods for treating a disease in a subject by modifying a target nucleic acid associated with a gene or expression of a gene related to the disease. In some embodiments, methods comprise administering a composition or cell described herein to a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include, but are not limited to the diseases and syndromes listed in TABLE 5.

TABLE 5
DISEASES AND SYNDROMES
Exemplary Diseases and Syndromes

11-hydroxylase deficiency; 17,20-desmolase deficiency; 17-hydroxylase deficiency; 3-
hydroxyisobutyrate aciduria; 3-hydroxysteroid dehydrogenase deficiency; 46,XY gonadal
dysgenesis; AAA syndrome; ABCA3 deficiency; ABCC8-associated hyperinsulinism;
aceruloplasminemia; acromegaly; achondrogenesis type 2; acral peeling skin syndrome;
acrodermatitis enteropathica; adrenocortical micronodular hyperplasia;
adrenoleukodystrophies; adrenomyeloneuropathies; Aicardi-Goutieres syndrome; Alagille
disease (also called Alagille Syndrome); Alexander Disease, Alpers syndrome; alpha-1
antitrypsin deficiency (AATD); alpha-mannosidosis; Alstrom syndrome; Alzheimer's disease;
amebic dysentery; amelogenesis imperfecta; amish type microcephaly; amyotrophic lateral
sclerosis (ALS); anaplastic large cell lymphoma; anauxetic dysplasia; androgen insensitivity
syndrome; angiopathic thrombosis; antiphospholipid syndrome; Antley-Bixler syndrome;
APECED, Apert syndrome, aplasia of lacrimal and salivary glands, argininemia,
arrhythmogenic right ventricular dysplasia, Arts syndrome, ARVD2, arylsulfatase deficiency
type metachromatic leokodystrophy, ataxia telangiectasia, autoimmune lymphoproliferative
syndrome; autoimmune polyglandular syndrome type 1; autosomal dominant anhidrotic
ectodermal dysplasia; autosomal dominant deafness; autosomal dominant polycystic kidney
disease; autosomal recessive microtia; autosomal recessive renal glucosuria; autosomal
visceral heterotaxy; babesiosis; balantidial dysentery; Bardet-Biedl syndrome; Bartter
syndrome; basal cell nevus syndrome; Batten disease; benign recurrent intrahepatic
cholestasis; beta-mannosidosis; β-thalassemia; Bethlem myopathy; Blackfan-Diamond
anemia; bleeding disorder (coagulation); blepharophimosis; Byler disease; C syndrome;
CADASIL; calcific aortic stenosis; calcification of joints and arteries; carbamyl phosphate
synthetase deficiency; cardiofaciocutaneous syndrome; Carney triad; carnitine
palmitoyltransferase deficiencies; cartilage-hair hypoplasia; cblC type of combined
methylmalonic aciduria; CD18 deficiency; CD3Z-associated primary T-cell
immunodeficiency; CD40L deficiency; CDAGS syndrome; CDG1A; CDG1B; CDG1M;
CDG2C; CEDNIK syndrome; central core disease; centronuclear myopathy; cerebral capillary
malformation; cerebrooculofacioskeletal syndrome type 4; cerebrooculogacioskeletal
syndrome; cerebrotendinous xanthomatosis; Chaga's Disease; Charcot Marie Tooth Disesase;
cherubism; CHILD syndrome; chronic granulomatous disease; chronic recurrent multifocal
osteomyelitis; citrin deficiency; classic hemochromatosis; CNPPB syndrome; cobalamin C
disease; Cockayne syndrome; coenzyme Q10 deficiency; Coffin-Lowry syndrome; Cohen
syndrome; combined deficiency of coagulation factors V; common variable immune
deficiency 3; complement hyperactivation; complete androgen insentivity; cone rod
dystrophies; conformational diseases; congenital bile adid synthesis defect type 1; congenital
bile adid synthesis defect type 2; congenital defect in bile acid synthesis type; congenital
erythropoietic porphyria; congenital generalized osteosclerosis; Cornelia de Lange syndrome;
coronary heart disease; Cousin syndrome; Cowden disease; COX deficiency; Cri du chat
syndrome; Crigler-Najjar disease; Crigler-Najjar syndrome type 1; Crisponi syndrome;
Crouzon syndrome; Currarino syndrome; Curth-Macklin type ichthyosis hystrix; cutis laxa;
cystic fibrosis; cystinosis; d-2-hydroxyglutaric aciduria; DDP syndrome; Dejerine-Sottas
disease; Denys-Drash syndrome; Dercum disease; desmin cardiomyopathy; desmin myopathy;
DGUOK-associated mitochondrial DNA depletion; diabetes Type I; diabetes Type II;
disorders of glutamate metabolism; distal spinal muscular atrophy type 5; DNA repair
diseases; dominant optic atrophy; Doyne honeycomb retinal dystrophy; Dravet Syndrome;
Duchenne muscular dystrophy; dyskeratosis congenita; Ehlers-Danlos syndrome type 4;
Ehlers-Danlos syndromes; Elejalde disease; Ellis-van Creveld disease; Emery-Dreifuss
muscular dystrophies; encephalomyopathic mtDNA depletion syndrome; encephalitis;
enzymatic diseases; EPCAM-associated congenital tufting enteropathy; epidermolysis bullosa
with pyloric atresia; epilepsy; facioscapulohumeral muscular dystrophy; Factor V Leiden
thrombophilia; Faisalabad histiocytosis; familial atypical mycobacteriosis; familial capillary
malformation-arteriovenous; Familial Creutzfeld-Jakob disease; familial esophageal achalasia;
familial glomuvenous malformation; familial hemophagocytic lymphohistiocytosis; familial
mediterranean fever; familial megacalyces; familial schwannomatosis; familial spina bifida;
familial splenic asplenia/hypoplasia; familial thrombotic thrombocytopeniaurpura; Fanconi
disease (Fanconi anemia); Feingold syndrome; FENIB; fibrodysplasia ossificans progressiva;
FKTN; Fragile X syndrome; Francois-Neetens fleck corneal dystrophy; Frasier syndrome;
Friedreich's ataxia; FTDP-17; Fuchs corneal dystrophy; fucosidosis; G6PD deficiency;
galactosialidosis; Galloway syndrome; Gardner syndrome; Gaucher disease; Gitelman
syndrome; GLUT1 deficiency; GM2- Gangliosidoses (e.g., Tay Sachs Disease, Sandhoff
Disease) glycogen storage disease type 1b; glycogen storage disease type 2; glycogen storage
disease type 3; glycogen storage disease type 4; glycogen storage disease type 9a; glycogen
storage diseases; GM1-gangliosidosis; Greenberg syndrome; Greig cephalopolysyndactyly
syndrome; hair genetic diseases; hairy cell leukemia; HANAC syndrome; harlequin type
ichtyosis congenita; HDR syndrome; hearing loss; hemochromatosis type 3; hemochromatosis
type 4; hemolytic anemia; hemolytic uremic syndrome; hemophilia A; hemophilia B;
hereditary angioedema type 3; hereditary angioedemas; hereditary hemorrhagic telangiectasia;
hereditary hypofibrinogenemia; hereditary intraosseous vascular malformation; hereditary
leiomyomatosis and renal cell cancer; hereditary neuralgic amyotrophy; hereditary sensory and
autonomic neuropathy type; Hermansky-Pudlak disease; HHH syndrome; HHT2; hidrotic
ectodermal dysplasia type 1; hidrotic ectodermal dysplasias; histiocytic sarcoma; HNF4A-
associated hyperinsulinism; HNPCC; homozygous familial hypercholesterolemia; human
immunodeficiency with microcephaly; human papilloma virus (HPV) infection; Huntington's
disease; hyper-IgD syndrome; hyperinsulinism-hyperammonemia syndrome;
hypercholesterolemia; hypertrophy of the retinal pigment epithelium; hypochondrogenesis;
hypohidrotic ectodermal dysplasia; ICF syndrome; idiopathic congenital intestinal pseudo-
obstruction; immunodeficiency 13; immunodeficiency 17; immunodeficiency 25;
immunodeficiency with hyper-IgM type 1; immunodeficiency with hyper-IgM type 3;
immunodeficiency with hyper-IgM type 4; immunodeficiency with hyper-IgM type 5;
immunoglobulin alpha deficiency; inborn errors of thyroid metabolism; infantile
myofibromatosis; infantile visceral myopathy; infantile X-linked spinal muscular atrophy;
intrahepatic cholestasis of pregnancy; IPEX syndrome; IRAK4 deficiency; isolated congenital
asplenia; Jeune syndrome; Johanson-Blizzard syndrome; Joubert syndrome; JP-HHT
syndrome; juvenile hemochromatosis; juvenile hyalin fibromatosis; juvenile nephronophthisis;
Kabuki mask syndrome; Kallmann syndromes; Kartagener syndrome; KCNJ11-associated
hyperinsulinism; Kearns-Sayre syndrome; Kostmann disease; Kozlowski type of
spondylometaphyseal dysplasia; Krabbe disease; LADD syndrome; late infantile-onset
neuronal ceroid lipofuscinosis; LCK deficiency; LDHCP syndrome; Leber Congenital
Amaurosis Teyp 10; Legius syndrome; Leigh syndrome; lethal congenital contracture
syndrome 2; lethal congenital contracture syndromes; lethal contractural syndrome type 3;
lethal neonatal CPT deficiency type 2; lethal osteosclerotic bone dysplasia; leukocyte adhesion
deficiency; Li Fraumeni syndrome; LIG4 syndrome; lipodystrophy; lissencephaly type 1;
lissencephaly type 3; Loeys-Dietz syndrome; low phospholipid-associated cholelithiasis;
Lynch Syndrome; lysinuric protein intolerance; a lysosomal storage disease (e.g., Hunter
syndrome, Hurler syndrome); macular dystrophy; Maffucci syndrome; Majeed syndrome;
mannose-binding protein deficiency; mantle cell lymphoma; Marfan disease; Marshall
syndrome; MASA syndrome; mastocytosis; MCAD deficiency; McCune-Albright syndrome;
MCKD2; Meckel syndrome; MECP2 Duplication Syndrome; Meesmann corneal dystrophy;
megacystis-microcolon-intestinal hypoperistalsis; megaloblastic anemia type 1; MEHMO;
MELAS; Melnick-Needles syndrome; MEN2s; meningitis; Menkes disease; metachromatic
leukodystrophies; methymalonic acidemia due to transcobalamin receptor defect;
methylmalonic acidurias; methylvalonic aciduria; microcoria-congenital nephrosis syndrome;
microvillous atrophy; migraine; mitochondrial neurogastrointestinal encephalomyopathy;
monilethrix; monosomy X; mosaic trisomy 9 syndrome; Mowat-Wilson syndrome;
mucolipidosis type 2; mucolipidosis type Ma; mucolipidosis type IV; mucopolysaccharidoses;
mucopolysaccharidosis type 3A; mucopolysaccharidosis type 3C; mucopolysaccharidosis type
4B; multiminicore disease; multiple acyl-CoA dehydrogenation deficiency; multiple cutaneous
and mucosal venous malformations; multiple endocrine neoplasia type 1; multiple sulfatase
deficiency; mycosis fungoides; myotonic dystrophy; NAIC; nail-patella syndrome; nemaline
myopathies; neonatal diabetes mellitus; neonatal surfactant deficiency; nephronophtisis;
Netherton disease; neurofibromatoses; neurofibromatosis type 1; Niemann-Pick disease type
A; Niemann-Pick disease type B; Niemann-Pick disease type C; NKX2E; non-alcoholic fatty
liver disease (NAFLD); non-alcoholic steatohepatitis (NASH); Noonan syndrome; North
American Indian childhood cirrhosis; NROB1 duplication-associated DSD; ocular genetic
diseases; oculo-auricular syndrome; OLEDAID; oligomeganephronia; oligomeganephronic
renal hypolasia; Ollier disease; Opitz-Kaveggia syndrome; orofaciodigital syndrome type 1;
orofaciodigital syndrome type 2; osseous Paget disease; osteogenesis imperfecta;
otopalatodigital syndrome type 2; OXPHOS diseases; palmoplantar hyperkeratosis; panlobar
nephroblastomatosis; Parkes-Weber syndrome; Parkinson's disease; partial deletion of
21q22.2-q22.3; Pearson syndrome; Pelizaeus-Merzbacher disease; Pendred syndrome;
pentalogy of Cantrell; peroxisomal acyl-CoA-oxidase deficiency; Peutz-Jeghers syndrome;
Pfeiffer syndrome; Pierson syndrome; pigmented nodular adrenocortical disease; pipecolic
acidemia; Pitt-Hopkins syndrome; plasmalogens deficiency; platelet glycoprotein IV
deficiency; pleuropulmonary blastoma and cystic nephroma; polycystic kidney disease;
polycystic ovarian disease; polycystic lipomembranous osteodysplasia; Pompe disease,
including infantile onset Pompe disease (IOPD) and late onset Pompe disease (LOPD);
porphyrias; PRKAG2 cardiac syndrome, premature ovarian failure; primary erythermalgia;
primary hemochromatoses; primary hyperoxaluria; progressive familial intrahepatic
cholestasis; propionic acidemia; protein-losing enteropathy; pyruvate decarboxylase
deficiency; RAPADILINO syndrome; renal cystinosis; retinitis pigmentosa; Rett Syndrome;
rhabdoid tumor predisposition syndrome; Rieger syndrome; ring chromosome 4; Roberts
syndrome; Robinow-Sorauf syndrome; Rothmund-Thomson syndrome; severe combined
immunodeficiency disorder (SCID); Saethre-Chotzen syndrome; Sandhoff disease; SC
phocomelia syndrome; SCAS; Schinzel phocomelia syndrome; short rib-polydactyly
syndrome type 1; short rib-polydactyly syndrome type 4; short-rib polydactyly syndrome type
2; short-rib polydactyly syndrome type 3; Shwachman disease; Shwachman-Diamond disease;
sickle cell anemia; Silver-Russell syndrome; Simpson-Golabi-Behmel syndrome; Smith-
Lemli-Opitz syndrome; SPG7-associated hereditary spastic paraplegia; spherocytosis;
spinocerebellar ataxia; split-hand/foot malformation with long bone deficiencies;
spondylocostal dysostosis; sporadic visceral myopathy with inclusion bodies; storage diseases;
Stargardt macular dystrophy; STRA6-associated syndrome; stroke; Tay-Sachs disease;
thanatophoric dysplasia; thyroid metabolism diseases; Tourette syndrome; transthyretin-
associated amyloidosis; trisomy 13; trisomy 22; trisomy 2p syndrome; tuberous sclerosis;
tufting enteropathy; urea cycle diseases; Usher Syndrome; Van Den Ende-Gupta syndrome;
Van der Woude syndrome; variegated mosaic aneuploidy syndrome; VLCAD deficiency; von
Hippel-Lindau disease; von Willebrand disease; Waardenburg syndrome; WAGR syndrome;
Walker-Warburg syndrome; Werner syndrome; Wilson disease; Wiskott-Aldrich Syndrome;
Wolcott-Rallison syndrome; Wolfram syndrome; X-linked agammaglobulinemia; X-linked
chronic idiopathic intestinal pseudo-obstruction; X-linked cleft palate with ankyloglossia; X-
linked dominant chondrodysplasia punctata; X-linked ectodermal dysplasia; X-linked Emery-
Dreifuss muscular dystrophy; X-linked lissencephaly; X-linked lymphoproliferative disease;
X-linked visceral heterotaxy; xanthinuria type 1; xanthinuria type 2; xeroderma pigmentosum;
XPV; and Zellweger disease.

In some embodiments, compositions and methods modify at least one gene associated with the disease or the expression thereof. In some embodiments, the disease is Alzheimer's disease and the gene is selected from APP, BACE-1, PSD95, MAPT, PSEN1, PSEN2, and APOEE4. In some embodiments, the disease is Parkinson's disease and the gene is selected from SNCA, GDNF, and LRRK2. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINA1. In some embodiments, the disease is amyotrophic lateral sclerosis (ALS) and the gene is selected from SOD1, FUS, C90RF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises calcific aortic stenosis and the gene is Apo(a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMR1. In some embodiments, the disease comprises Fuchs corneal dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe disease (IOPD) and late onset Pompe disease (LOPD) and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCIII, APOA1, APOL1, ARH, CDKN2B, CFB, CXCL12, FXI, FXII, GATA-4, MIA3, MKL2, MTHFD1L, MYH7, NKX2-5, NOTCH1, PKK, PCSK9, PSRC1, SMAD3, and TTR. In some embodiments, the disease comprises acromegaly and the gene is GHR. In some embodiments, the disease comprises acute myeloid leukemia and the gene is CD22. In some embodiments, the disease is diabetes and the gene is GCGR. In some embodiments, the disease is NAFLD/NASH and the gene is selected from DGAT2 and PNPLA3. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne muscular dystrophy and the gene is DMD. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot Marie Tooth disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMEM43. In some embodiments, the disease comprises Factor V Leiden thrombophilia and the gene is F5. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld-Jakob disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVIII. In some embodiments, the disease comprises histiocytosis and the gene is CD1. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHI1, NPHP1, CEP290, TMEM67, RPGRIP1L, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TP53. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease is mycosis fungoides and the gene is CD7. In some embodiments, the disease is myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COL1A1, COL1A2, and IFITM5. In some embodiments, the disease is non-small cell lung cancer and the gene is selected from KRAS, EGFR, ALK, METex14, BRAF V600E, ROS1, RET, and NTRK. In some embodiments, the disease comprises Peutz-Jeghers syndrome and the gene is STK11. In some embodiments, the disease comprises polycystic kidney disease and the gene is selected from PKD1 and PKD2. In some embodiments, the disease comprises Severe Combined Immune Deficiency and the gene is selected from IL7R, RAG1, JAK3. In some embodiments, the disease comprises PRKAG2 cardiac syndrome and the gene is PRKAG2. In some embodiments, the disease comprises spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease comprises Usher Syndrome and the gene is selected from MYO7A, USH1C, CDH23, PCDH15, USH1G, USH2A, GPR98, DFNB31, and CLRN1. In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46.

In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

### Cancer

In some embodiments, the disease is cancer. In some embodiments, the cancer is a solid cancer (i.e., a tumor). In some embodiments, the cancer is selected from a blood cell cancer, a leukemia, and a lymphoma. The cancer can be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL). In some embodiments, the cancer is any one of colon cancer, rectal cancer, renal-cell carcinoma, liver cancer, bladder cancer, cancer of the kidney or ureter, lung cancer, non-small cell lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, and thyroid cancer.

In some embodiments, mutations are associated with cancer or are causative of cancer. The target nucleic acid, in some embodiments, comprises a portion of a gene comprising a mutation associated with a disease, such as cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or a combination thereof. Non-limiting examples of genes comprising a mutation associated with a disease such as cancer are ABL, ACE, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Cα1, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDC1, PDGFRA, PHOX2B, PIM-1, PMS2, POLD1, POLE, POT1, PPARG, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP3, TMEM127, TNF, TP53, TRAC, TSC1, TSC2, TRK, VHL, WRN, and WT1. Non-limiting examples of oncogenes are KRAS, NRAS, BRAF, MYC, CTNNB1, and EGFR. In some embodiments, the oncogene is a gene that encodes a cyclin dependent kinase (CDK). Non-limiting examples of CDKs are Cdk1, Cdk4, Cdk5, Cdk7, Cdk8, Cdk9, Cdk11 and CDK20. Non-limiting examples of tumor suppressor genes are TP53, RB1, and PTEN.

### Infections

Described herein are compositions and methods for treating an infection in a subject. Infections may be caused by a pathogen, e.g., bacteria, viruses, fungi, and parasites. Compositions and methods may modify a target nucleic acid associated with the pathogen or parasite causing the infection. In some embodiments, the target nucleic acid may be in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, methods described herein include treating an infection caused by one or more viral pathogens. Non-limiting examples of viral pathogens include adenovirus, blue tongue virus, chikungunya, coronavirus (e.g., SARS-CoV-2), cytomegalovirus, Dengue virus, Ebola, Epstein-Barr virus, feline leukemia virus, Hemophilus influenzae B, Hepatitis virus A, Hepatitis virus B, Hepatitis virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, methods described herein include treating an infection caused by one or more parasites. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### XVI. Cells

Disclosed herein, in some aspects, are cells comprising a target nucleic acid that has been modified with a composition, system or method disclosed herein. Cells described herein may also be referred to as genetically modified cells. Cells may be eukaryotic (e.g., a mammalian cell). Cells may be prokaryotic. Cells may be obtained from a multicellular organism. Cells may be derived from a multicellular organism (e.g., a cell line). Cells may comprise a heritable genetic mutation, such that progeny cells derived therefrom comprise the heritable genetic mutation. In some instances, cells are progeny of a genetically modified cell comprising a genetic modification of the genetically modified parent cell. A genetically modified cell may comprise a deletion, insertion, mutation, or non-native sequence relative to a wild-type version of the cell or the organism from which the cell was derived.

Non-limiting examples of cells that may be engineered or modified with compositions, systems and methods described herein include immune cells, such as CAR T-cells, B-cells, NK cells, granulocytes, basophils, eosinophils, neutrophils, mast cells, monocytes, macrophages, dendritic cells, antigen-presenting cells (APC), or adaptive cells. In general, CAR T-cell refers to a T cell that has a nucleotide sequence encoding a chimeric antigen receptor (CAR). Non-limiting examples of cells that may be engineered or modified with compositions and methods described herein include plant cells, such as parenchyma, sclerenchyma, collenchyma, xylem, phloem, germline (e.g., pollen). Cells from lycophytes, ferns, gymnosperms, angiosperms, bryophytes, charophytes, chlorophytes, rhodophytes, or glaucophytes. Non-limiting examples of cells that may be engineered or modified with compositions and methods described herein include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

In some instances, cells are in a subject. Compositions of the disclosure may be administered to a subject. A subject may be a human. A subject may be a mammal (e.g., rat, mouse, cow, dog, pig, sheep, horse). A subject may be a vertebrate or an invertebrate. A subject may be a laboratory animal. A subject may be a patient. A subject may be suffering from a disease. A subject may display symptoms of a disease. A subject may not display symptoms of a disease, but still have a disease. A subject may be under medical care of a caregiver (e.g., the subject is hospitalized and is treated by a physician). Methods of the disclosure may be performed in a plant, bacteria, or a fungus.

Cells may be modified by compositions, systems or methods described herein under various conditions. Cells may be modified in vitro. Cells may be modified in vivo. Cells may be modified ex vivo. A cell may be an isolated cell. A cell may be a cell inside of or on an organism. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. A cell may be a prokaryotic cell or derived from a prokaryotic cell. A cell may be a bacterial cell or may be derived from a bacterial cell. A cell may be an archaeal cell or derived from an archaeal cell. A cell may be a eukaryotic cell or derived from a eukaryotic cell. A cell may be a pluripotent stem cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell. A cell may be a microbe cell or derived from a microbe cell. A cell may be a fungi cell or derived from a fungi cell. A cell may be from a specific organ or tissue.

Cells include cell lines modified by composition, systems or methods disclosed herein. Cell lines may be used to produce a desired protein. In some instances, the cell line is a Chinese hamster ovary cell line (CHO), human embryonic kidney cell line (HEK), cell lines derived from cancer cells, cell lines derived from lymphocytes, and the like. Non-limiting examples of cell lines includes: 132-d5 human fetal fibroblasts, 10.1 mouse fibroblasts, 293-T, 3T3, 3T3 Swiss, 3T3-L1, 721, 9L, A-549, A10, A172, A20, A253, A2780, A2780ADR, A2780cis, A375, A431, ALC, ARH-77, B16, B35, BALB/3T3 mouse embryo fibroblast, BC-3, BCP-1 cells, BEAS-2B, BHK-21, BR 293, BS-C-1 monkey kidney epithelial, Bcl-1, bEnd.3, BxPC3, C3H-10T1/2, C6/36, C8161, CCRF-CEM, CHO, CHO Dhfr −/−, CHO-7, CHO-IR, CHO-K1, CHO-K2, CHO-T, CIR, CML T1, CMT, COR-L23, COR-L23/5010, COR-L23/CPR, COR-L23/R23, COS, COS-1, COS-6, COS-7, COS-M6A, COV-434, CT26, CTLL-2, CV1, CaCo2, Cal-27, Calu1, D17, DH82, DLD2, DU145, DuCaP, EL4, EM2, EM3, EMT6/AR1, EMT6/AR10.0, FM3, H1299, H69, HASMC, HB54, HB55, HB56, HCA2, HEK-293, HEKa, HEKn, HL-60, HMEC, HT-29, HUVEC, HeLa, HeLa B, HeLa T4, HeLa-S3, Hep G2, Hepa1c1c7, Huh1, Huh4, Huh7, IC21, J45.01, J82, JY cells, Jurkat, Jurkat, K562 cells, KCL22, KG1, KYO1, Ku812, LNCap, LRMB, MC-38, MCF-10A, MCF-7, MDA-MB-231, MDA-MB-435, MDA-MB-468, MDCK II, MDCK II, MEF, mIMCD-3C8161, MOLT, MONO-MAC 6, MOR/0.2R, MRC5, MTD-1A, Ma-Mel 1-48, MiaPaCell, MyEnd, NALM-1, NCI-H69/CPR, NCI-H69/LX10, NCI-H69/LX20, NCI-H69/LX4, NHDF, NIH-3T3, NRK, NRK-52E, NW-145, OPCN/OPCT cell lines, P388D1, PC-3, PNT-1A/PNT 2, Panc1, Peer, RIN-5F, RMA/RMAS, RPTE, Rat6, Raw264.7, RenCa, SEM-K2, SK-UT, SKOV3, SW480, SW620, Saos-2 cells, Sf-9, SkBr3, T-47D, T2, T24, T84, TF1, THP1 cell line, TIB55, U373, U87, U937, VCaP, Vero cells, WEHI-231, WM39, WT-49, X63, YAC-1, and YAR.

Cells include cell lines modified by composition, systems or methods disclosed herein, wherein the cell line is derived from a eukaryote, also referred to as a eukaryotic cell line. In some instances, the eukaryotic cell is a Chinese hamster ovary (CHO) cell. In some instances, the eukaryotic cell is a Human embryonic kidney 293 cells (also referred to as HEK or HEK 293) cell. Non-limiting examples of cell lines that may be used with compositions, systems and methods of the present disclosure include C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR.

Non-limiting examples of cells that may be used with the disclosure include immune cells, such as T-cells, B-cells, NK cells, granulocytes, basophils, eosinophils, neutrophils, mast cells, monocytes, macrophages, dendritic cells, antigen-presenting cells (APC), and adaptive cells. Non-limiting examples of cells that may be used with this disclosure also include plant cells, such as Parenchyma, sclerenchyma, collenchyma, xylem, phloem, germline (e.g., pollen). Cells from lycophytes, ferns, gymnosperms, angiosperms, bryophytes, charophytes, chlorophytes, rhodophytes, or glaucophytes. Non-limiting examples of cells that may be used with this disclosure also include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

### XVII. Agricultural Engineering

Compositions and methods of the disclosure may be used for agricultural engineering. For example, compositions and methods of the disclosure may be used to confer desired traits on a plant. A plant may be engineered for the desired physiological and agronomic characteristic using the present disclosure. In some instances, the target nucleic acid sequence comprises a nucleic acid sequence of a plant. In some instances, the target nucleic acid sequence comprises a genomic nucleic acid sequence of a plant cell. In some instances, the target nucleic acid sequence comprises a nucleic acid sequence of an organelle of a plant cell. In some instances, the target nucleic acid sequence comprises a nucleic acid sequence of a chloroplast of a plant cell.

The plant may be a dicotyledonous plant. Non-limiting examples of orders of dicotyledonous plants include Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales.

The plant may be a monocotyledonous plant. Non-limiting examples of orders of monocotyledonous plants include Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales. A plant may belong to the order, for example, Gymnospermae, Pinales, Ginkgoales, Cycadales, Araucariales, Cupressales and Gnetales.

Non-limiting examples of plants include plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses, wheat, maize, rice, millet, barley, tomato, apple, pear, strawberry, orange, acacia, carrot, potato, sugar beets, yam, lettuce, spinach, sunflower, rape seed, Arabidopsis, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. A plant may include algae.

### EXAMPLES

The following examples are included for illustrative purposes only and are not intended to limit the scope of the invention.

### Example 1: PAM Screening for Effector Proteins

Effector proteins described herein, including effector proteins comprising an amino acid sequences selected from SEQ ID NOS: 1-1614 and 3195-3302, are screened by in vitro enrichment (IVE) for PAM recognition. Briefly, effector proteins are complexed with guide RNAs for about 15 minutes at about 37° C. The complexes are added to an IVE reaction mix. PAM screening reactions use about 10 μl of RNP in 100 μl reactions with about 1,000 ng of a 5′ PAM library in 1x Cutsmart buffer and are carried out for about 15 minutes at about 25° C., or about 45 minutes at about 37° C., or about 15 minutes at about 45° C. Reactions are terminated with proteinase K and EDTA for about 30 minutes at about 37° C. Next generation sequencing is performed on cut sequences to identify enriched PAMs.

### Example 2: DETECTR Activity of Effector Proteins

Effector proteins are tested for trans cleavage. Briefly, partially purified (e.g., nickel-NTA purified) effector proteins are incubated with crRNA and tracrRNA (or an sgRNA) in a trans cleavage buffer (e.g., 20 mM Tricine, 15 mM MgCl2, 0.2 mg/ml BSA, 1 mM TCEP (pH 9 at 37° C.)) at room temperature for about 20 minutes, followed by addition of target nucleic acid at a final concentration of 10 nM to produce effector-protein guide complexes. Trans cleavage activity is detected by fluorescence signal upon cleavage of a fluorophore-quencher reporter in a DETECTR reaction. Dilutions of the effector-protein guide complexes are performed, and the assay repeated at 1%, 0.1% or 0.01% of the original protein concentration. The dilution that provided the highest signal ratio is listed.

### Example 3: Effector Proteins Edit Genomic DNA in Mammalian Cells

Effector proteins are tested for their ability to produce indels in a mammalian cell line (e.g., HEK293T cells). Briefly, a plasmid encoding the effector proteins and a guide RNA are delivered by lipofection to the mammalian cells. This is performed with a variety of guide RNAs targeting several loci adjacent to biochemically determined PAM sequences. Indels in the loci are detected by next generation sequencing of PCR amplicons at the targeted loci and indel percentage is calculated as the fraction of sequencing reads containing insertions or deletions relative to an unedited reference sequence. Sequencing libraries with less than 20% of reads aligning to the reference sequence are excluded from the analysis for quality control purposes. “No plasmid” and Cas9 are included as negative and positive controls, respectively.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-103. (canceled)

104. A composition comprising an effector protein and an engineered guide nucleic acid, wherein the effector protein comprises an amino acid sequence that is at least 90% identical to any one of SEQ ID NOs: 1-1614 and 3195-3302.

105. The composition of claim 104, wherein the amino acid sequence of the effector protein is at least 90% identical to a sequence selected from Column A1 of TABLE 1; and at least a portion of the engineered guide nucleic acid comprises a nucleobase sequence that is at least 90% identical to a sequence selected from Column B1 of TABLE 1, wherein the sequence from Column A1 and the sequence from Column B1 are in the same row of TABLE 1.

106. The composition of claim 104, wherein the amino acid sequence of the effector protein is at least 90% identical to a sequence selected from Column A2 of TABLE 1; and at least a portion of the engineered guide nucleic acid comprises a nucleobase sequence that is at least 90% identical to a sequence selected from Column B2 of TABLE 1, wherein the sequence from Column A2 and the sequence from Column B2 are in the same row of TABLE 1.

107. The composition of claim 104, wherein the amino acid sequence of the effector protein is at least 90% identical to a sequence selected from Column A3 of TABLE 1; and at least a portion of the engineered guide nucleic acid comprises a nucleobase sequence that is at least 90% identical to a sequence selected from Column B3 of TABLE 1, wherein the sequence from Column A3 and the sequence from Column B3 are in the same row of TABLE 1.

108. The composition of claim 104, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column C1 of TABLE 2; and at least a portion of the engineered guide nucleic acid comprises a nucleobase sequence that is at least 90% identical to a sequence selected from Column D1 of TABLE 2, wherein the sequence from Column C1 and the sequence from Column D1 are in the same row of TABLE 2.

109. The composition of claim 104, wherein the amino acid sequence of the effector protein is at least 90% identical to a sequence selected from Column C2 of TABLE 2; and at least a portion of the engineered guide nucleic acid comprises a nucleobase sequence that is at least 90% identical to a sequence selected from Column D2 of TABLE 2, wherein the sequence from Column C2 and the sequence from Column D2 are in the same row of TABLE 2.

110. The composition of claim 104, wherein the effector protein comprises a nuclear localization signal.

111. The composition of claim 104, comprising a donor nucleic acid.

112. The composition of claim 104, comprising a fusion partner protein.

113. The composition of claim 112, wherein the fusion partner protein is directly fused to the N terminus or C terminus of the effector protein.

114. The composition of claim 112, wherein the fusion partner protein comprises a polypeptide selected from a deaminase, a transcriptional activator, a transcriptional repressor, or a functional domain thereof.

115. The composition of claim 104, wherein the effector protein is a catalytically inactive nuclease.

116. A nucleic acid expression vector encoding the effector protein and the engineered guide nucleic acid of claim 104, optionally wherein the nucleic acid expression vector is an adeno-associated viral vector.

117. A pharmaceutical composition, comprising the composition of claim 104, and a pharmaceutically acceptable excipient.

118. A system, comprising the composition of claim 104, and at least one detection reagent for detecting a target nucleic acid.

119. A method of modifying a target nucleic acid in a sample, comprising:
contacting the sample with the composition of claim 104, thereby generating a modification of the target nucleic acid; and
optionally detecting the modification.

120. A method of detecting a target nucleic acid in a sample, comprising:
contacting the sample with the composition of claim 104, and a reporter nucleic acid comprising a detectable moiety that produces a detectable signal in the presence of the target nucleic acid and the composition or system; and
detecting the detectable signal.

121. A method of modifying a target nucleic acid, comprising: contacting the target nucleic acid with the composition of claim 104, thereby modifying the target nucleic acid.

122. A cell comprising the composition of claim 104.

123. A method of treating a disease comprising administering to a subject in need thereof the composition of claim 104.
